(19) United States
(12) Patent Application Publication
(10) Pub. No.: US 2004/0197793 A1
(43) Pub. Date: Oct. 7, 2004

(54) Methods and apparatus for biomolecule detection, identification, quantification and/or sequencing

(76) Inventors: Arjang Hassibi, Palo Alto, CA (US); Babek Hassibi, San Marino, CA (US); Siavash Ghazvini, Menlo Park, CA (US)

Correspondence Address: Blakely, Sokoloff, Taylor & Zafman, Seventh Floor, 12400 Wilshire Boulevard, Los Angeles, CA, 90025-1030, US

(21) Appl. No.: 10/627,557

(22) Filed: Jul. 24, 2003

Related U.S. Application Data
(60) Provisional application No. 60/407,412, filed on Aug. 30, 2002.
(60) Provisional application No. 60/422,439, filed on Oct. 29, 2002.
(60) Provisional application No. 60/435,924, filed on Dec. 20, 2002.
(60) Provisional application No. 60/435,934, filed on Dec. 20, 2002.
(60) Provisional application No. 60/440,670, filed on Jan. 15, 2003.
(60) Provisional application No. 60/451,107, filed on Feb. 27, 2003.
(60) Provisional application No. 60/470,347, filed on May 13, 2003.

(51) Int. Cl. C12Q 1/68; C12Q 1/66

(52) U.S. Cl. 435/6; 435/8

(57) ABSTRACT

The present invention concerns methods, compositions and apparatus for detecting. Identifying, quantifying and/or sequencing target biomolecules, such as nucleic acids or proteins. Where the target biomolecule is not a nucleic acid, the target or a ligand that binds to the target may be tagged with an oligonucleotide or nucleic acid. The presence of target molecules in samples may be detected by a variety of enzymatic processes that generate a detectable product, such as pyrophosphate (PPi) or ATP. In preferred embodiments of the invention, the product is detected by a bioluminescence regenerative cycle (BRC), utilizing luciferase mediated bioluminescence. In other preferred embodiments, thermostable enzymes may be used in either isothermal or cyclic thermal reactions, such as terminal transferase activity or nucleic acid polymerization, to generate PPi. Apparatus and compositions for biomolecule analysis are also disclosed. Methods for analysis of generated data are also disclosed herein.

The present application claims the benefit under 35 U.S.C. § 119(e) of provisional Patent Application Serial No. 60/407,412, filed Aug. 30, 2002; No. 60/422,439, filed Oct. 19, 2002; No. 60/435,924, filed Dec. 20, 2002; No. 60/435,934, filed Dec. 20, 2002; 60/440,670, filed Jan. 15, 2003; No. 60/451,107, filed Feb. 27, 2003; and No. 60/470,347, filed May 13, 2002, entitled, “Nucleic Acid Detection and Quantification Using Terminal Transferase Based. Assays” by Arjang Hassibi and Siavash Ghazvini. The text of each provisional application is incorporated herein by reference in its entirety.

### BACKGROUND OF THE INVENTION

1. Field of the Invention

The present invention relates to the field of molecular biology. More specifically, the invention relates to methods, compositions and apparatus for biomolecule detection, identification, quantification and/or sequencing. In certain embodiments of the invention, the biomolecules may be nucleic acids, proteins, peptides, antibodies and/or any other biomolecule that can be tagged with a nucleic acid and/or oligonucleotide. In particular embodiments of the invention, the methods may involve use of a bioluminescence regenerative cycle (BRC) and optical detection of bioluminescence.

2. Description of Related Art

Methods of precise and highly sensitive detection, identification, quantification and/or sequencing of biomolecules, such as nucleic acids or proteins, are of use for a number of medical, forensic, epidemiological, public health, biological warfare and other applications. A variety of molecular biology and genomic techniques would benefit from the availability of precise and sensitive methods for biomolecule detection, identification, quantification and/or sequencing.

Existing methods of nucleic acid analysis include use of hybridization based assays, such as DNA arrays, real time PCR™, single nucleotide polymorphism (SNP) analysis and DNA sequencing techniques. DNA microarrays provide a platform for exploring the genome, including analysis of gene expression by hybridization with sequence specific oligonucleotide probes attached to chips in precise arrays. (E.g., Schena et al., Science 270:467-470, 1995; Shalon et al., Genome Res. 6:639-645, 1996; Pease et al., Proc. Natl. Acad. Sci. USA 91:5022-26, 1994). Microarray technology is an extension of previous hybridization-based methods, such as Southern and Northern blotting, that have been used to identify and quantify nucleic acids in biological samples (Southern, J. Mol. Biol. 98:503-17, 1975; Pease et al., Proc. Natl. Acad. Sci. USA 93:10614-19, 1996). Identification of a target nucleic acid in a sample typically involves fluorescent detection of the nucleic acid hybridized to an oligonucleotide at a particular location on the array. Fluorescent detection may be too insensitive to detect very low levels of a target nucleic acid in a sample. It is also more qualitative than quantitative. Thus, detection of small changes in the level of expression of a particular gene, as might be attempted for high through-put screening of potential inhibitors and/or activators of gene expression, may not be feasible using a fluorescence detection system with microarrays. More accurate and sensitive methods for nucleic acid detection, identification and/or quantification are needed.

Real time PCR™ (e.g., Model 770 TaqMan® system, Applied Biosystems, Foster City, Calif.) is another technique for which accurate and sensitive nucleic acid detection and/or quantification methods are needed. Typically, if the target of interest is present, it will be amplified by replication using flanking primers and a nucleic acid polymerase. A probe, which may consist of a complementary oligonucleotide with attached reporter and quencher dyes, is designed to bind to the amplified target nucleic acid between the two primer-binding sites. The nuclease activity of the polymerase cleaves the probe, resulting in an increase in fluorescence of the reporter dye after it is separated from the quencher. PCR™ based fluorescence detection of target nucleic acids is more sensitive, due to the amplification effect of the technique. However, precise quantification of the amount of target present may be complicated by a variety of factors, such as contaminating nuclease activity or variability in the efficiency of amplification.

Single nucleotide polymorphisms (SNPs) are of increasing interest in molecular biology, genomics and disease diagnostics. SNP detection may be used for haplotype construction in genetic studies to identify and/or detect genes associated with various disease states, as well as drug sensitivity or resistance. SNPs may be detected by a variety of techniques, such as DNA sequencing, fluorescence detection, mass spectrometry or DNA microarray hybridization (e.g., U.S. Pat. Nos. 5,885,775; 6,368,799). Existing methods of SNP detection may suffer from insufficient sensitivity or an unacceptably high level of false positive and/or false negative results. A need exists for more sensitive and accurate methods of detecting SNPs.

Pyrophosphate based detection systems have been used for DNA sequencing (e.g., Nyren and Lundin, Anal. Biochem. 151:504-509, 1985; U.S. Pat. Nos. 4,971,903; 6,210,891; 6,258,568; 6,274,320, each incorporated herein by reference). The method uses a coupled reaction wherein pyrophosphate is generated by an enzyme-catalyzed process, such as nucleic acid polymerization. The pyrophosphate is used to produce ATP, in an ATP sulfurylase catalyzed reaction with adenosine 5′-phosphosulphate (APS). The ATP in turn is used for the production of light in a luciferin-luciferase coupled reaction. However, the “pyrosequencing” technique is based on sequential addition of single nucleotides, in the presence of nucleotide degrading enzymes to remove any unincorporated nucleotides (U.S. Pat. Nos. 6,210,891 and 6,258,568). This results in low levels of light emission, with relatively low sensitivity, and requires a complex and expensive apparatus to perform the assay. Alternative methods of DNA sequencing, such as the standard Sanger dideoxy sequencing technique, may involve the use of radioisotopes or other toxic chemicals that pose difficult disposal problems. Such methods also may be relatively insensitive and typically can only sequence short segments of nucleic acids in a single run—typically 500 basepairs or less. Determination of longer sequences requires repetitive sequencing and compilation of overlapping sequences. This makes standard nucleic acid sequencing techniques tedious, expensive, time-consuming and labor intensive.

Certain embodiments of the present invention involve detection, identification and/or quantification of target proteins, peptides or other biomolecules that are tagged with reporter oligonucleotides and/or nucleic acids. A number of methods are known for protein identification, detection and quantification, such as SDS-polyacrylamide gel electrophoresis, capillary electrophoresis, limited proteolysis and tandem array mass spectrometry, enzyme assay, cell-based assays and a wide of immunological techniques such as Western blotting and ELISA. In certain instances, such techniques may require partial or even full purification of the protein of interest before it can be quantified. In other cases, the detection methods, such as immunoassay, may show cross-reactivity with other proteins that may be present in a complex mixture. Immunoassays also require that one or more antibodies be prepared against the target protein of interest, a laborious and time-consuming process. Improved methods for detection, identification, quantification and/or sequencing of biomolecules, such as nucleic acids or oligonucleotide-tagged proteins, peptides, etc. are needed. Preferably such methods would be simple, inexpensive and rapid, with high sensitivity and specificity for the target molecule to be detected.

### SUMMARY OF THE INVENTION

The present invention fulfills an unresolved need in the art by providing methods for accurately detecting, identifying, quantifying and/or sequencing target biomolecules, such as nucleic acids or proteins. In preferred embodiments, the number of target biomolecules in a sample may be accurately determined over a seven order of magnitude range. The disclosed methods provide increased sensitivity and accuracy of target biomolecule detection, identification, quantification and/or sequencing compared to prior art methods. Other advantages include lower cost, decreased use of toxic chemicals and avoidance of radioisotopes, decreased sample preparation and more rapid analysis.

In certain embodiments of the invention, the methods may comprise obtaining at least one sample suspected of containing one or more target biomolecules. Where the target of interest is a nucleic acid, it may be captured and/or isolated by a variety of known techniques, such as sequence specific hybridization with one or more capture probes. Alternatively, the nucleic acid content of a sample may be partially or fully isolated by known techniques, such as differential extraction, precipitation, ultrafiltration, ultracentrifugation, chromatography, enzymatic digestion of contaminants and/or solid phase binding, such as binding to nylon or nitrocellulose membranes or to magnetic beads. Captured nucleic acids may be assayed as disclosed below. In other alternative embodiments, target nucleic acids may be analyzed, detected and/or quantified in solution phase, for example using sequence specific primers designed to only hybridize with selected target nucleic acids.

In other embodiments of the invention, the target may comprise an oligonucleotide and/or nucleic acid tag attached to a biomolecule, such as a protein, peptide, antibody, antigen, enzyme, binding protein, ligand, substrate and/or inhibitor. The target may be captured and/or isolated using known techniques, such as antibody-antigen binding, protein-ligand binding, enzyme-inhibitor or enzyme-substrate binding, solid phase binding, etc. and the tag detected as disclosed below. Alternatively, solution phase assays could also be used for target biomolecules other than nucleic acids. The oligonucleotide and/or nucleic acid tag may be detected as disclosed below, for example by BRC assay.

In some embodiments of the invention, target proteins or other biomolecules may be detected by binding to an aptamer. Aptamers are oligonucleotides that exhibit specific binding interactions that are not based on standard Watson-Crick basepair formation. Aptamers are therefore similar to antibodies in their binding characteristics. Aptamers may be derived by an in vitro evolutionary process called SELEX (e.g., Brody and Gold, Molecular Biotechnology 74:5-13, 2000). Aptamers are relatively small molecules on the order of 7 to 50 kDa that may be produced by known methods (e.g., U.S. patent Nos. U.S. Pat. Nos. 5,270,163; 5,567,588; 5,670,637; 5,696,249; 5,843,653) or obtained from commercial sources. (e.g, Somalogic, Boulder, Colo.). Because they are small, stable and not as easily damaged as proteins, they may be well suited for assays involving binding to the surface of a solid matrix. Because aptamers may be comprised of DNA, they can serve as substrates for terminal transferase or other enzymatic activity as disclosed herein. In certain alternative embodiments of the invention, the presence of target biomolecules other than nucleic acids may be assayed by binding an aptamer or an aptamer-bligonucleotide conjugate to a target biomolecule and then assaying for the presence of the aptamer or conjugate, for example using a BRC process.

The captured and/or isolated targets may be detected, identified and/or quantified using a variety of enzymatic assays. In certain embodiments of the invention, terminal transferase may be used to detect, identify and/or quantify target biomolecules. However, the skilled artisan will realize that a variety of enzyme based detection techniques may be utilized within the scope of the present invention, so long as the enzyme produces a product (e.g., pyrophosphate, ATP, ADP, AMP, GTP, etc.) that can be assayed. Other enzymes that may be coupled to bioluminescent detection include DNA polymerases, RNA polymerases, reverse transcriptases, adenylate kinase, phosphoenolpyruvate kinase, and many other enzymes known in the art. In preferred embodiments of the invention, the enzyme coupled assay system produces pyrophosphate (PPi) and/or ATP. As discussed in more detail below, in more preferred embodiments bioluminescent detection may utilize a luciferin/luciferase coupled assay system, such as BRC.

In preferred embodiments of the invention, target nucleic acids and/or oligonucleotides coupled to target biomolecules may be detected, identified and/or quantified using a bioluminescence regenerative cycle (BRC) assay, discussed in more detail below. The BRC process may be used to detect reaction products from a variety of enzymes. For example, terminal transferase may be added to a nucleic acid or oligonucleotide in the presence of nucleotides (dNTPs). Terminal transferase will add nucleotides to the 3′ end of single-stranded DNA (ssDNA) or the 3′ overhangs of double-stranded DNA that has been treated, for example, with a restriction endonuclease. Terminal transferase may also add nucleotides to blunt-ended double-stranded DNA or the recessed 3′ ends of restricted double-stranded DNA, with lower efficiency. Incorporation of nucleotides by terminal transferase results in generation of pyrophosphate (PPi), with one molecule of PPi generated for each nucleotide incorporated. The skilled artisan will realize that the terminal transferase reaction is exemplary only and that many other enzymes, such as DNA or RNA polymerases, can also generate PPi by incorporation of nucleotides into DNA or RNA strands.

In certain embodiments of the invention, the pyrophosphate producing reaction is allowed to proceed to completion before BRC analysis. Once the reaction is complete, the pyrophosphate is reacted with APS (adenosine 5′-phosphosulfate) in the presence of ATP sulfurylase to produce ATP and sulphate. The ATP is reacted with oxygen and luciferin in the presence of luciferase to yield oxyluciferin, AMP and pyrophosphate. The PPi may react again with APS to regenerate ATP. For each molecule of pyrophosphate that is cycled through BRC, a photon of light is emitted with a quantum efficiency of 0.88 and one molecule of pyrophosphate is regenerated. Because of the relative kinetic rates of luciferase and ATP sulfurylase, a steady state is reached in which the concentrations of ATP and pyrophosphate and the level of photon output remain relatively constant over an extended period of time. The number of photons may be counted (integrated) over a time interval to determine the number of target nucleic acids in the sample. The very high sensitivity of BRC is related in part to the integration of light output over time, in contrast to other methods that measure light output at a single time point or at a small number of fixed time points. The ability to vary the length of time over which photon integration occurs also contributes to the very high and controllable dynamic range for nucleic acid molecule quantification, with a sensitivity of detection as low as 0.1 attomoles (amol). Increasing the length of integration also significantly reduces detection noise.

In preferred embodiments of the invention, steady state light output is subjected to data analysis involving integration of light output over a time interval, providing an accurate and highly sensitive method of quantifying the number of target biomolecules in the sample. In various embodiments of the invention, light output by BRC may be corrected for background light emission (for example, by PPi contaminating one or more reagents) by comparing enzyme (e.g., terminal transferase) mediated photon emission with the background photon emission.

In other alternative embodiments of the invention, PPi generation may be assayed in real time as the PPi is produced. PPi may be reacted with APS to produce ATP, which can generate light via a luciferin/luciferase process as discussed above. Rather than reaching a steady state, light output may increase with time as an enzyme-coupled reaction produces an increasing concentration of PPi. The light output curve may be subjected to kinetic analysis to determine the amount of target biomolecule present in the sample. Such a process may exhibit increased sensitivity of detection by maximizing the amount of light output generated for a given amount of target biomolecule. In various embodiments the BRC assay may be modified to increase light output, for example by utilizing a super BRC assay, a branched BRC assay, a rolling circle BRC assay or a transcription based branched BRC assay as disclosed in more detail below.

In certain embodiments of the invention, thermostable enzymes may be used in a BRC or other detection method. Thermostable forms of terminal transferase, ATP sulfurylase and luciferase are disclosed herein and may be used for either isothermal processes or thermal cycling reactions. Thermostable forms of polymerases, such as Taq polymerase are known in the art and may be utilized in the disclosed methods.

In certain embodiments of this invention, to reduce the background signal of the assay caused by ATP and/or PPi contamination, ATP and PPi degrading enzymes, and or reagents may be used before the BRC procedure. After sufficient background reduction, the enzyme and/or reagent can be extracted or deactivated by physical or chemical means, resulting in a contamination free reaction solution for BRC assays. For instance apyrase (ATP-diphosphatase EC 3.6.1.5, Smartt et. al. 1995) can be used to degrade contaminating ATP, while pyrophosphatase (EC 3.6.1.1, Cooperman et. al. 1992) may be used to degrade contaminating PPi molecules. Inactivation of these enzymes prior to BRC assay may be carried out by heating (e.g. 2 min above 80° C.), which does not effect thermostable BRC enzymes.

The invention is not limited to use of ATP-Sulfurylase as the enzyme converting PPi to ATP. Other enzymes may be used to create the regenerative cycle as well (e.g., Heinonen, “Biological Role of Inorganic Pyrophosphate”, Kluwer Academic Publishers, 2001) if they are able to synthesis ATP out of PPi by consuming other substrates. Non-limiting examples of such enzymes are listed in Table 1 below.

TABLE 1
Exemplary ATP Producing Enzymes

| Enzyme | Reaction | Reference |
|---|---|---|
| FMN Adenyltransferase | PPi + FAD [symbol] ATP + FMN | Schrerer and |
| [EC 2.7.7.2] | | Kornberg 1950 |
| Adenylyl Transferase | PPi + NAD⁺ [symbol] ATP + | Kornberg 1948 |
| [EC 2.7.7.1] | nicotinamide ribonucleotide | |
| Glucose-1-Phosphate | PPi + ADP-glucose [symbol] ATP + | Munch- |
| Adenyltransferase | α-D-glucose-1-phosphate | Petersen et al. |
| [EC 2.7.7.27] | | 1953 |

The invention is not limited to BRC assay of enzyme activity. It will be apparent to the skilled artisan that many different methods of assaying enzyme activity are known and may be used in the practice of the disclosed methods, such as incorporation of fluorescently tagged nucleotides and fluorescence spectroscopy; incorporation of radioactively tagged nucleotides and liquid scintillation counting or other radioassay; incorporation of Raman labels and Raman spectroscopy; incorporation of NMR labels and nuclear magnetic resonance assay, and many other techniques known in the art. In various embodiments of the invention, multi-color detection methods may be employed, using nucleotides tagged with different colored fluorophores.

In some embodiments of the invention, the disclosed methods are of use for a wide variety of applications for which target biomolecule detection, identification, quantification and/or sequencing is desired. Such applications include, but are not limited to, measuring gene expression levels, detecting and/or quantifying pathogens in a sample, performing real-time PCR™ analysis, detecting single nucleotide polymorphisms (SNPs) and/or nucleic acid sequence analysis.

In various embodiments of the invention, the number of target proteins or peptides in a sample may be accurately determined over a wide concentration range. The disclosed methods provide increased sensitivity and accuracy of target molecule quantification compared to prior art methods. In preferred embodiments, the activity of the BRC process is initially inhibited by the presence of a selected peptide covalently or non-covalently attached to one or more of the BRC enzymes, such as luciferase or ATP sulfurylase. Removal of the inhibitory peptide by a protein or peptide of interest, present in a sample to be analyzed, initiates the light emitting BRC reactions. In some embodiments the inhibitory peptide may be removed by a target protease in the sample. In other cases, another type of target protein or peptide may act to restore activity of the inhibited BRC enzyme.

In certain embodiments of the invention, the methods may comprise obtaining at least one sample suspected of containing one or more target proteins and/or peptides, initiating pyrophosphate generation by activating one or more BRC enzymes, producing light by a bioluminescence regenerative cycle, accumulating the total number of photons produced over different time intervals, comparing the photon accumulation with the background photon emission and determining the number of target proteins and/or peptides in the sample

In other embodiments of the invention, a target protease may be covalently or non-covalently attached to another molecule to be quantified, such as another protein, peptide or other ligand. The protein, peptide or ligand may be indirectly quantified, by detecting the attached protease. Such protease tagged ligands may be used, for example, to quantify protein-protein binding interactions or any other type of known ligand-receptor binding interaction. The methods are not limited by the type of target protease used, including but not limited to a serine protease, a cysteine protease, an aspartic protease, a metallo-protease, a cathepsin, a collagenase, an elastase, kallikrein, plasmin, renin, streptokinase, subtilisin, thermolysin, thrombin, urokinase, HIV protease, trypsin, chymotrypsin, pepsin, gastrin, calcium-dependent proteases, magnesium-dependent proteases, proteinase K, papain, bromelain, or any other protease known in the art. The specificities of various proteases for different target peptide sequences are well known in the art. In certain embodiments, the presence or amount of a specific protease in a sample may be diagnostic for a disease state, such as cancer or hemophilia. In other embodiments, the presence of a bacterial or viral encoded protease in a sample, such as HIV protease or streptokinase, may be diagnostic for the presence of an infection with a pathogenic organism.

Other embodiments of the invention concern compositions and/or apparatus of use for assaying biomolecules. In an exemplary embodiment, an apparatus of use may comprise one or more of the following components: reaction chambers for BRC or other enzymatic process and/or target biomolecule capture; microfluidic system to add reagents or extract products from the reaction chamber(s); magnetic capture devices; vibration generator and/or mixing apparatus; optical coupling means to convey photons to a photodetector; photodetectors; sensor arrays; cooling and/or heating apparatus to control reaction chamber, photodetector and/or sensor temperature; temperature control module and/or data acquisition and analysis system. In exemplary embodiments, a cooled CCD camera imaging system or luminometer may be used as optical detectors, although any other optical detector known in the art may be used. In embodiments where a photodetector with a single fixed aperture of limited field is employed, the apparatus may optionally comprise a stage and/or motion control system to move the photodetector relative to a series of samples, for example a 96 well microtiter plate or other sample holder. The embodiments of the invention are not limited to photodetection and any other type of detector known in the art may be utilized.

In other embodiments of the invention, the apparatus may comprise one or more monodirectional microfluidic flow components, such as a cassette containing channels and/or microchannels. The cassette may comprise one or more sealed chambers connected by a monodirectional flow, with each sealed chamber containing a specific affinity matrix to capture a target biomolecule. A sample may pass through the cassette and be exposed to each chamber in turn, allowing binding of multiple target biomolecules to capture probes located in the chambers. After washing, the BRC detection reagents or other detection system reagents may be added and a signal, such as a bioluminescent signal, detected from each individual chamber. The chamber may be incorporated into a photodetection device or may be separately reacted with a sample and then inserted into a photodetection system. Many alternative forms of such a cassette system are known in the art and may be used, for example a microfluidic or capillary chip system as discussed in more detail below.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates an exemplary method for BRC. Nucleic acid polymerization results in the production of pyrophosphate, which is converted to ATP by ATP sulfurylase and APS. The ATP is broken down to pyrophosphate and AMP by luciferin/luciferase with a resulting emission of visible light. The pyrophosphate is recycled to regenerate ATP, resulting in an increase in steady-state luminescence. In alternative embodiments of the invention, other pyrophosphate generating enzyme-mediated processes besides nucleic acid polymerization may be assayed by BRC. In other alternative embodiments of the invention, other enzymes besides ATP sulfurylase may be utilized to recycle PPi to ATP.

FIG. 2 shows a bioluminescence regenerative cycle block diagram of exemplary ATP sulfurylase and luciferase catalyzed reactions in BRC.

FIG. 3 shows a simulation of a comparison between luciferase generated light intensity in the presence and absence of ATP sulfurylase and APS at different starting concentrations of ATP (luciferin=0.1 mM, APS=0.1 mM), based on the kinetic properties of the enzymes.

FIG. 4 illustrates an exemplary method for branched chain BRC assay.

FIG. 5 illustrates an exemplary method for transcription based branched chain BRC assay.

FIG. 6 illustrates an exemplary method for bioluminescence super regenerative cycle (super BRC) assay.

FIG. 7. illustrates exemplary methods of terminal transferase based assays, involving capture and detection of a nucleic acid target (1a-3a) or sandwich immunassay using a nucleic acid or oligonucleotide attached to an antibody, followed by extension of the 3′ terminus using terminal transferase.

FIG. 8 illustrates an exemplary apparatus for use with BRC detection.

FIG. 9 shows an exemplary result of a BRC assay, comparing light emission from a 0.1 pmol sample with a reference standard.

FIG. 10 shows the increase in steady state light emission from a 10 fmol (femtomole) sample. Random noise in the light emission can be filtered out by detecting a steady-state change in the baseline level of light emission.

FIG. 11 Photon generation by BRC assay. Photon intensity (photon/sec) was measured using a CCD imaging system with a 96-well microtiter plate format. (a) The target nucleic acid comprised 10 amol to 1 fmol of a 230 bp PCR product (Maltose binding protein). (b) The target nucleic acid comprised a single-stranded 40 bp oligo-loop, hybridized to itself, ranging in concentration from 1 fmol to 100 fmol. (c) The graph illustrates the quantitative results obtained, showing the dynamic range of the assay.

FIG. 12 Relative luminescence units measured by luminometer. Results normalized to a 1 fmol to 1 amol dilution series (incorporated dNTPs) for (a) ATP, (b) 40 bp oligo-loop and (c) 230 bp PCR product (Maltose binding protein).

FIG. 13(a) Taqman results from three dilution series of 10 ng of S. invicta Queen GP-9B expression. (b) Relative luminescence units measured from 1 ng of the same target nucleic acid with BRC.

FIG. 14 Relative luminescence from 40 μl of BRC reaction buffer using different dilutions of lysate from (a) U937 macrophage cells and (b) E. coli.

FIG. 15 shows an exemplary embodiment of BRC applied to SNP detection.

FIG. 16 shows an exemplary embodiment of BRC applied to pathogen detection.

FIG. 17 shows an exemplary embodiment of BRC using a rolling circle technique.

FIG. 18 shows an exemplary embodiment of BRC applied to measurement of protein-protein binding. One protein of the binding pair is labeled with a target oligonucleotide.

FIG. 19 shows an exemplary embodiment of BRC applied to measurement of gene expression.

FIG. 20 illustrates the use of BRC to detect complex genomic DNA inserted into a plasmid vector, with and without amplification of the target nucleic acid sequence. Detection and quantification of a target RO 52 insert sequence was demonstrated.

FIG. 21 illustrates exemplary hypothetical waveforms for each of the bases adenine (A), guanine (G), cytosine (C) and thymine (T) that would be detected during DNA sequencing.

FIG. 22 illustrates an exemplary hypothetical waveform generated for an exemplary DNA sequence TCTAGCTCAG (SEQ ID NO:6).

FIG. 23 illustrates a noise-corrupted aggregate waveform obtained from a uniformly asynchronous reaction of 10⁵ molecules of DNA with the exemplary sequence TCTAGCTCAG (SEQ ID NO:6).

FIG. 24 illustrates a reconstructed waveform using the Wiener solution (SNR_{perect}=40 db).

FIG. 25 illustrates a reconstructed waveform using the Wiener solution (SNR_{perfect}=35 db).

FIG. 26 illustrates a reconstructed waveform using the Wiener solution (SNR_{perect}=30 db).

FIG. 27 illustrates a reconstructed waveform using the Wiener solution (SNR_{perfect}=40 db and N=10⁶).

FIG. 28 shows an exemplary noise-corrupted aggregate waveform of. 10⁵DNA molecules with Gaussian delay distribution.

FIG. 29 illustrates an exemplary reconstructed waveform using the Wiener solution when the delay distribution is Gaussian (SNR_{perfect}=40 db).

FIG. 30 illustrates a schematic diagram of a photodetector consisting of a photodiode and an integrator with output potential for both high and low illumination.

### DESCRIPTION OF ILLUSTRATIVE EMBODIMENTS

Definitions

Terms that are not otherwise defined herein are used in accordance with their plain and/ordinary meaning.

As used herein, “a” or “an” may mean one or more than one of an item.

As used herein, the terms “analyte”, “biomolecule” and “target” mean any compound, molecule or aggregate of interest for detection. Non-limiting examples of targets include a nucleoside, nucleotide, oligonucleotide, polynucleotide, nucleic acid, peptide, polypeptide, protein, carbohydrate, polysaccharide, glycoprotein, lipid, hormone, growth factor, cytokine, receptor, antigen, allergen, antibody, substrate, metabolite, cofactor, inhibitor, drug, pharmaceutical, nutrient, toxin, poison, explosive, pesticide, chemical warfare agent, biowarfare agent, biohazardous agent, infectious agent, prion, radioisotope, vitamin, heterocyclic aromatic compound, carcinogen, mutagen, narcotic, amphetamine, barbiturate, hallucinogen, waste product, contaminant, heavy metal or any other molecule or atom, without limitation as to size, “Targets” are not limited to single molecules or atoms, but may also comprise complex aggregates, such as a virus, bacterium, Salmonella, Streptococcus, Legionella, E. coli, Giardia, Cryptosporidium, Rickettsia, spore, mold, yeast, algae, amoebae, dinoflagellate, unicellular organism, pathogen or cell. In certain embodiments, cells exhibiting a particular characteristic or disease state, such as a cancer cell, may be targets. Virtually any chemical or biological compound, molecule or aggregate could be a target, so long as it can be attached to a nucleic acid, polynucleotide or oligonucleotide, including but not limited to binding to an aptamer.

“Nucleic acid” means either DNA, RNA, single-stranded, double-stranded or triple stranded and any chemical modifications thereof. Virtually any modification of the nucleic acid is contemplated by this invention. “Nucleic acid” encompasses, but is not limited to, oligonucleotides and polynucleotides. Within the practice of the present invention, a “nucleic acid” may be of any length.

“Protein” is used herein to refer to any polymer comprised of amino acids, chemically modified amino acids, amino acid analogues and/or amino acid derivatives. The term “protein” encompasses amino acid polymers of any length, from two amino acid residues up to a full length protein. As used herein, the term “protein” encompasses, but is not limited to, peptides, oligopeptides and polypeptides.

BRC Detection

Various embodiments of the invention concern novel methods for quantifying target biomolecules without labeling of any target, capture or probe molecules. Such label free methods are advantageous with respect to sensitivity, expense and ease of use. In certain embodiments, the BRC methods involve the luminescent detection of pyrophosphate (PPi) molecules released from an enzyme-catalyzed reaction, such as RNA or DNA polymerization or terminal transferase catalyzed nucleotide addition. As part of the technique, a bioluminescence regenerative cycle (BRC) is triggered by the release of inorganic pyrophosphate (PPi).

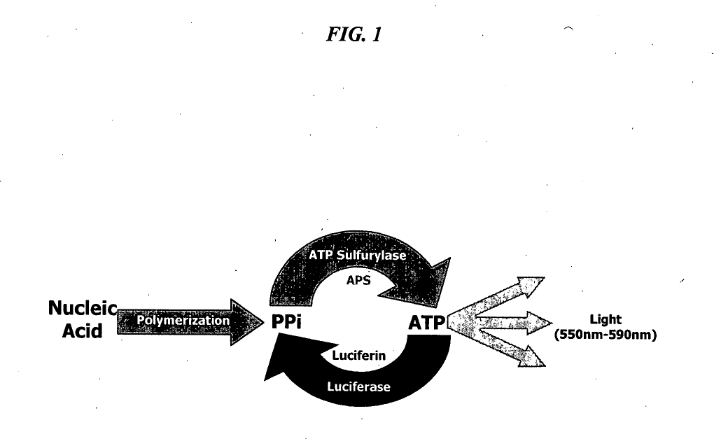

The regenerative cycle is illustrated in FIG. 1. It involves a first reaction of PPi with APS, catalyzed by ATP-sulfurylase enzyme, which results in the production of ATP and inorganic sulphate. In a second reaction, luciferin and luciferase consume ATP as an energy source to generate light, AMP and oxyluciferin and to regenerate PPi (FIG. 1). Thus, after each BRC cycle, a quantum of light is generated for each molecule of PPi in solution, while the net concentration of ATP in solution remains relatively stable and is proportional to the initial concentration of PPi. In the course of the reactions, APS and luciferin are consumed and AMP and oxyluciferin are generated, while ATP sulfurylase and luciferase remain constant. The invention is not limited as to the type of luciferase used. Although certain disclosed embodiments utilized firefly luciferase, any luciferase known in the art may be used in the disclosed methods.

As a result of the BRC process illustrated in FIG. 1, where the enzyme mediated production of PPi is completed before initiation of bioluminescence, the photon emission rate remains steady and is a monotonic function of the amount of PPi in the initial mixture. For very low concentrations of PPi (10⁻⁸ M or less), the total number of photons generated in a fixed time interval is proportional to the number of PPi molecules. Where PPi is generated by the polymerase catalyzed replication of a target nucleic acid, by terminal transferase mediated addition of nucleotides to the 3′ end of a target nucleic acid, or for any other enzyme mediated process where the amount of target biomolecule is a limiting factor, the number of photons generated in a fixed time interval is proportional to the quantity of the target biomolecule present in the sample.

The basic concept of enzymatic light generation from PPi molecules was introduced almost two decades ago (Nyren and Lundin, 1985; Nyren, Anal. Biochem. 167:235-238, 1987). Pyrophosphate based luminescence has been used for DNA sequencing (Ronaghi et al., Anal. Biochem. 242:84-89, 1996) and SNP detection (Nyren et al., Anal. Biochem. 244:367-373, 1997). The present methods provide additional procedures for accurately quantifying specific target nucleic acids in low density arrays or other systems, in the presence of contaminants and detector noise. The novel system and methods have an intrinsic controllable dynamic range up to seven orders of magnitude and are sensitive enough to detect target nucleic acids at attomole (10⁻¹⁸) or lower levels. Other features of the “pyrosequencing” method disclosed by Nyren and others include addition of a single type of nucleotide at a time, either sequentially or to separate reaction chambers, and addition of nucleotide degrading enzymes such as apyrase to the pyrosequencing reaction (see, e.g., U.S. Pat. Nos. 6,210,891 and 6,258,568). Such processes are designed to measure bioluminescent light emission as single light pulses of limited intensity and duration. Advantages of the BRC process disclosed herein include the attainment of steady-state light emission, allowing data accumulation by integration of photon emission over time, and amplification of photon emission by recycling of PPi to regenerate ATP.

Analysis of Steady State BRC

In various enzyme-catalyzed reactions, PPi molecules are generated when nucleotides (dNTPs or NTPs) are incorporated into a growing nucleic acid chain. For each addition of a nucleotide, one PPi molecule is cleaved from the dNTP by the enzyme (e.g. Klenow fragment of DNA polymerase I, RNA polymerase or terminal transferase) and released into the reaction buffer. The reactions catalyzed by DNA and RNA polymerases are shown in Eq. 1 and Eq. 2.

(DNA)ₙ+dNTP→(DNA)ₙ₊₁+PPi (1)

(RNA)ₙ+NTP→(RNA)ₙ₊₁+PPi (2)

If one assumes that the strand is completely polymerized, then the number of PPi molecules (N_{PPi}) released during the process is given by Eq. 3.

N_{PPi}=N_{NA}·(L_{NA}−L_{P}) (3)

Where N_{NA} is the total number of primed nucleic acid molecules present in the reaction buffer, and L_{NA} and L_{P} are respectively the lengths of the nucleic acid chain and the primer.

Enzymatic Bioluminescence Cycle

In preferred embodiments of the invention, photons may be generated from pyrophosphate by using ATP-sulfurylase (Ronesto et al., Arch. Biochem. Biophys. 290:66-78, 1994; Beynon et al. Biochemistry, 40, 14509-14517, 2001) to catalyze the transfer of the adenylyl group from APS to PPi, producing ATP and inorganic sulfate (Eq. 4).

PPi+APS⇄ATP+SO₄⁻² (4)

Next, luciferase catalyzes the slow consumption of ATP, luciferin and oxygen to generate a single photon (λₘₐₓ=562 nm, Q.E.≈0.88) per ATP molecule, regenerating a molecule of PPi and producing AMP, CO₂ and oxyluciferin (Eq. 5). (Brovko et al., Biochem. (Moscow) 59:195-201, 1994)

ATP+Luciferin+O₂→AMP+oxyluciferin+CO₂+hv+PPi (5)

Because the luciferase reaction is significantly slower than the ATP-sulfurylase reaction, in the presence of sufficient amounts of the substrates APS and luciferin a steady state cycle should be maintained, in which the concentration of ATP and the resulting levels of light emission remain relatively constant for a considerable time.

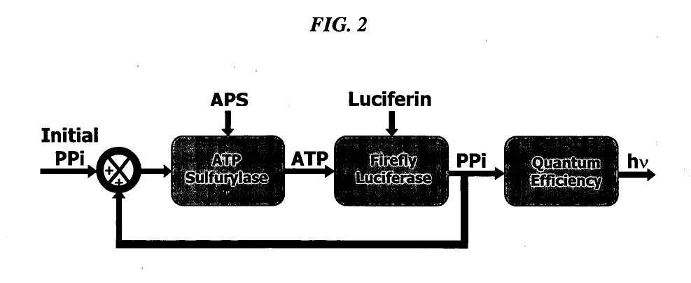

This steady state cycle is indicated schematically in FIG. 2. Because the steady-state photon emission is proportional to the initial concentration of PPi, the presence of minute amounts of PPi produced by a polymerase or other reaction should result in a detectable shift in baseline luminescence, even in the presence of considerable amounts of noise. The number of photons generated over time by the BRC cycle can potentially be orders of magnitude higher than the initial number of PPi molecules, which makes the system extremely sensitive compared to prior art methods. The increased sensitivity is provided by having a time-dependent amplification of light emission for each molecule of PPi present at the start of the BRC cycle, coupled with the ability to integrate photon emission over any selected time interval.

Photon Generation Rate

The photon generation rate of the system may be determined from the kinetics and steady state characteristics of the ATP sulfurylase and luciferase (Ronesto et al., 1994; Beynon et al., 2001; Brovko et al., 1994). In the presence of saturating concentrations of APS and luciferin, the ATP-sulfurylase reaction is orders of magnitude faster than the luciferase reaction. Thus, the rate of photon generation will be limited by the kinetics of luciferase rather than ATP-sulfurylase. A simplified equation expressing light intensity (I) in a unit volume for the BRC process is shown in Eq. 6.

$$I = α \frac{ⅆ}{ⅆ t} ( \frac{{N}_{ATP}}{V} ) = α \cdot ( \frac{{k}_{L}}{V} ) {N}_{ATP} \qquad ( 6 )$$

N_{ATP} is the number of ATP molecules in the solution, k_{L} is the turnover rate constant of luciferase, V is the volume of the solution, and a is the quantum efficiency of the bioluminescence process.

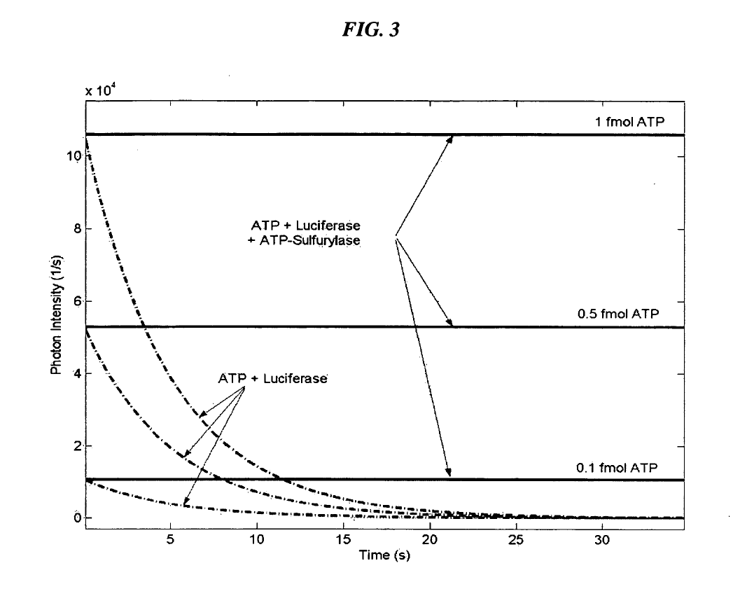

If ATP-sulfurylase was not present in the buffer, the light intensity would never reach a steady state and would simply decay as a function of time. In the presence of ATP-sulfurylase and APS, any decrease in the concentration of ATP will be compensated almost instantly by reaction of the generated PPi molecule with APS to regenerate ATP. This will cause the system to stay in a quasi-equilibrium state, where the concentrations of ATP and PPi remain relatively constant. At the same time, the luciferase reaction is constantly occurring and photons are emitted in a steady state fashion (FIG. 3). If the concentrations of APS and luciferin are high enough to assure saturation, then the steady state light intensity is given by Eq. 7.

$$I = α \cdot ( \frac{{k}_{L}}{V} ) {( {N}_{PP i} )}_{0} \qquad ( 7 )$$

(N_{PPi})₀ is the initial number of PPi molecules generated from the polymerization or other process. Combining equations 3 and 7 gives Eq. 8.

$$I = α \cdot ( \frac{{k}_{L}}{V} ) {N}_{NA} \cdot ( {L}_{NA} - {L}_{P} ) . \qquad ( 8 )$$

Equation 8 shows the proportionality between the generated light intensity and the initial number of nucleic acid molecules in a unit volume. If the number of photons detected is accumulated for a time interval T (integration time), the total number of photons generated (Nₚₕ) from the whole volume is given by Eq. 9.

Nₚₕ=α·k_{L}·T·N_{NA}·(L_{NA}−L_{P}) (9)

According to Eq. 9, the number of photons received by the detector (e.g. CCD camera) depends on the integration time and the number of target molecules present in the solution. By controlling the integration time the sensitivity of the system can be increased to any desired level limited by the saturation of the optical system. The dynamic range of the sensor system may therefore be proportionately enhanced.

Noise and Background Contamination

There are two phenomena that might potentially interfere with the performance and sensitivity of biomolecule detection. One is the possibility of PPi and/or ATP contamination from the chemicals included in the buffer solution. The other is the noise of the detector (e.g. thermal noise and/or shot noise in a photodiode system). The effects of ATP and PPi contamination on light emission may be modeled by modifying Eq. 8 to account for an initial existing number of PPi (and/or ATP) molecules C_{PPi}, resulting in Eq. 10.

$$I = α \cdot ( \frac{{k}_{L}}{V} ) \cdot [ {N}_{NA} \cdot ( {L}_{NA} - {L}_{P} ) + {C}_{PPi} ] . \qquad ( 10 )$$

Although C_{PPi} is relatively low for common bioluminescence measurements (on the order of 0.1 to 10 femtomoles), it can be an order of magnitude higher than the target biomolecule concentration. It is also possible to have variation between experiments in the value of C_{PPi} of as much as 300%. To eliminate the effects of any possible contamination, the light intensity of the system is initially measured in the absence of any PPi generated from polymerization. This serves as an initial reference point for measuring the catalytically produced PPi. If the light intensity in the reference state is Iᵣ, by combining equations 9 and 10 the value of N_{NA} may be calculated from Eq. 11.

$${N}_{NA} = ( \frac{V}{α \cdot {k}_{L}} ) \frac{I - {I}_{r}}{{L}_{NA} - {L}_{p}} \qquad ( 11 )$$

In terms of number of photons detected;

$${N}_{NA} = ( \frac{1}{α \cdot {k}_{L}} ) \frac{{N}_{p h} - {N}_{p hr}}{T \cdot ( {L}_{NA} - {L}_{p} )} \qquad ( 12 )$$

To account for the noise of the system, it is assumed that the total noise of the detector n(t) is random and has a normal distribution N(0,σ), with a mean of zero and a standard deviation of σ. Thus, the apparent light intensity in the presence of detector noise is given by Eq. 13.

$$I ( t ) = α \cdot ( \frac{{k}_{L}}{V} ) {N}_{NA} \cdot ( {L}_{NA} - {L}_{P} ) + n ( t ) , \qquad ( 13 )$$

Integrating Eq. 13 over a time interval T,

$${N}_{N A}^{′} = ( \frac{1}{α \cdot {k}_{L}} ) \frac{{N}_{p h} - {N}_{p hr} + {\int }_{T}^{} ( {n}_{1} ( τ ) - {n}_{2} ( τ ) ) ⅆ τ}{( {L}_{NA} - {L}_{p} ) \cdot T} \qquad ( 14 )$$

where n₁(t) and n₂(t) are the noise introduced by the detector in the actual experiment and reference respectively. n₁(t) and n₂(t) are uncorrelated but have the same normal distribution of N(0,σ). N′_{NA} is the measured nucleic acid quantity. Equation 14 can be rewritten as

N′_{NA}=N_{NA}+n′(t), (15)

where n′(t) is a normal distribution defined as

$${N}_{N A}^{′} - {N}_{N A} = {n}^{′} ( t ) \to N ( 0 , \sqrt{\frac{2}{T}} \cdot \frac{V σ}{α \cdot {k}_{L} ( {L}_{N A} - {L}_{p} )} ) \qquad ( 16 )$$

As shown in Eq. 16, the difference between the estimated and actual quantity of the target nucleic acid (measurement error) has a normal distribution. The standard deviation of error is a function of chemistry (k_{L} of luciferase in the assay), noise of the detector, and integration time. To achieve a selected level of error tolerance, the required integration time for a given chemistry and specific level of detector noise may be calculated.

The above analysis provides a quantitative basis for determination of the number of target nucleic acid (or other) molecules present in a sample, accounting for the presence of contaminants and noise in the system. The resulting method provides a highly sensitive and accurate procedure for determining the number of target molecules in a given sample. These methods are broadly applicable for a variety of techniques in which quantitative detection of target molecules is desired.

BRC Amplification Methods

In various embodiments of the invention, non-steady state BRC methods may be utilized to increase the signal strength (e.g., amplitude of photon emission) detected from a given concentration of a target biomolecule. Many alternative methods for amplifying the light emission signal detected by BRC may be utilized. Exemplary methods, discussed below, include branched chain BRC, transcription based BRC and super BRC.

Branched BRC Assay

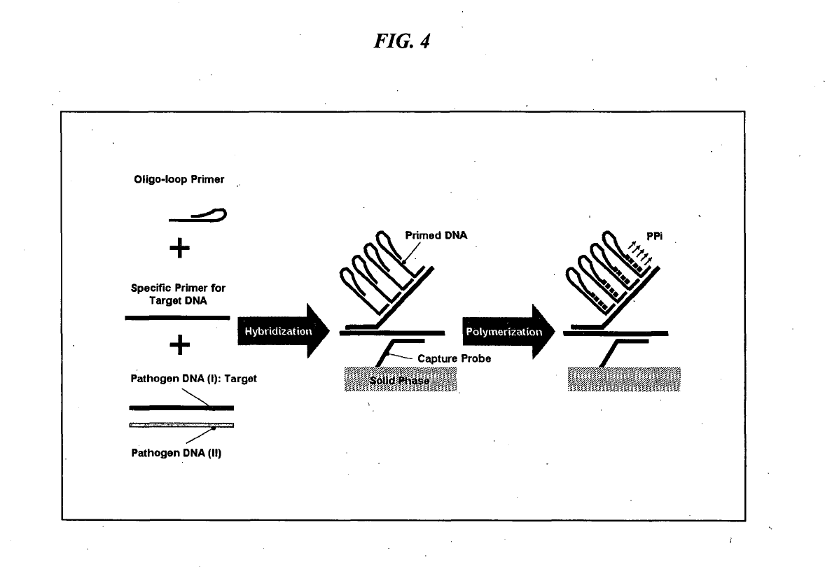

Pyrophosphate generation triggered by the presence of a target biomolecule is not limited to the extension of a primer on a target nucleic acid and/or oligonucleotide tag. An alternative to increase the amount of nucleic acid polymerization, and hence increase the amount of pyrophosphate generated, is to extend off of the primer itself (FIG. 4). This requires use of a first primer (target specific primer) that is partially complementary in sequence to the target nucleic acid and/or oligonucleotide tag and partially complementary in sequence to a second primer. The second primer (oligo-loop primer) is partially complementary in sequence to the first primer, and partially complementary in sequence to itself. The first primer is allowed to bind to the target DNA. In FIG. 4, the DNA target is indicated as a pathogen DNA. However, the method may be used with any target nucleic acid of at least partially known sequence. The second primer is allowed to hybridize to a different portion of the first primer. The second primer then hybridizes to itself. Upon addition of polymerase and nucleotides, the second primer essentially primes its own duplication (FIG. 4), generating pyrophosphate in the process. FIG. 4 also illustrates an exemplary embodiment wherein a capture probe is used to bind to the target molecule at attach it to a solid substrate.

This branching method can potentially generate thousands of pyrophosphate molecules per target biomolecule. The specificity of pyrophosphate generation is limited by the hybridization processes (capture probe, first primer and second primer), not the polymerization process. If the extendable bases in the branch complex (second primer) is equal to L_{B}, then the light intensity from the unit volume of the reaction buffer which contains Nₚ branch probes is

$$I = α \cdot ( \frac{{k}_{L}}{V} ) \cdot {N}_{P} \cdot {L}_{X} \qquad ( 17 )$$

Potentially, the branched chain method may be more sensitive than known methods, such as PCR™ amplification of the target itself.

Transcription-Based Branched BRC Assays

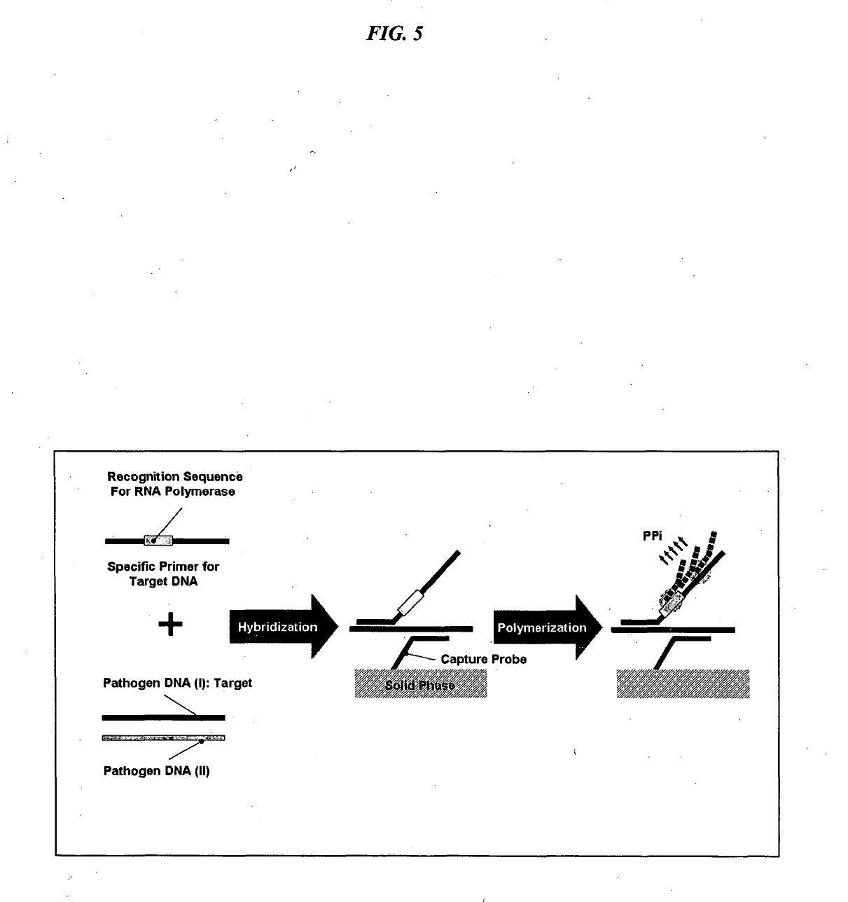

An alternative embodiment of the invention, transcription-based branched BRC assay (FIG. 5), is similar to the branched BRC method disclosed above. It differs in that instead of utilizing a self-complementary second primer, it incorporates a recognition site (promoter sequence) for RNA polymerase into the target specific primer (FIG. 5). The RNA polymerase recognition (promoter) sequence results in the generation of RNA molecules through the incorporation of nucleotides by RNA polymerase and therefore a steady generation of PPi molecules (FIG. 5). The method is not limiting for the type of polymerase utilized and could incorporate either prokaryotic or eukaryotic promoter sequences, to be used with a prokaryotic or eukaryotic RNA polymerase, respectively. Promoter sequences are well known in the art, as discussed further below.

The kinetics of the PPi generation in this method is a function of target (bound primer) quantity and may be detected by real-time monitoring of light by BRC. The photon generation rate in this system grows as a linear function of time and can be defined in a unit volume by:

$$I ( t ) = α \cdot ( \frac{{k}_{L}}{V} ) \cdot {k}_{t} \cdot {N}_{T} \cdot t , \qquad ( 18 )$$

where kₜ is the average turnover rate of the polymerization process of the probes and N_{T} is the total number of target molecules in the volume.

Bioluminescence Super Regenerative Cycle (BSRC) Assays

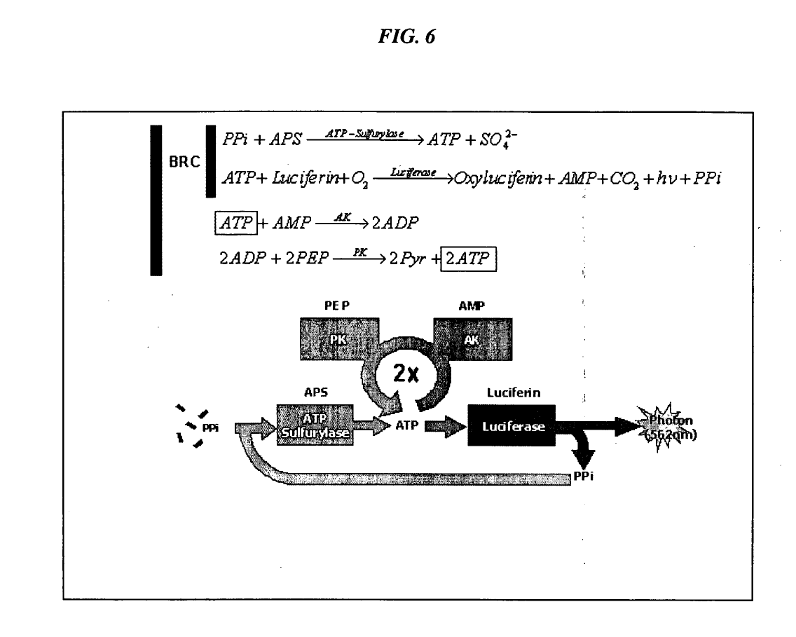

The two exemplary embodiments of the invention discussed above increase the sensitivity of BRC detection by generating pyrophosphate from replication of the primer sequence. A third exemplary embodiment, bioluminescence super regenerative cycle (BSRC, or “super BRC”) results in signal amplification through the generation of 2 ATP molecules for every pyrophosphate by utilizing an additional enzyme-coupled process. In the exemplary embodiment disclosed in FIG. 6, the additional enzymes are adenylate kinase and pyruvate kinase, with phosphoenolpyruvate added. However, the skilled artisan will realize that alternative combinations of enzymes and substrates could potentially be utilized to obtain the same result.

As shown in FIG. 6, the BRC enzymes are used to produce ATP from APS and PPi. The ATP may be reacted with AMP in the presence of adenylate kinase, producing two molecules of ADP. In this method an additional enzymatic complex is added to the standard BRC reaction: Adenylate Kinase (AK) in the presence of AMP substrate, and pyruvate kinase (PK) in the presence of phosphoenolpyruvate (PEP). The additional enzymes can create two ATP molecules from a single ATP by substrate cycling. Adenylate kinase catalyzes the transfer of a phosphate group from ATP to AMP, creating two molecules of ADP. Pyruvate kinase catalyzes the transfer of a phosphate group from PEP to ADP to form ATP, resulting in the creation of two molecules of ATP for every molecule of ATP previously present. This process would exponentially increase the concentration of ATP molecules in the reaction buffer. Since bioluminescence light activity of luciferase is proportional to the ATP concentration, the amount of light generated grows exponentially as a function of time. The rate of light generation growth depends on the kinetics of AK and PK and the concentration of their substrates.

The light intensity generated by the BSRC method, considering an exponential growth rate of k for the concentration of ATP molecules, is a function of time defined by

$$I = α \cdot ( \frac{{k}_{L}}{V} ) \cdot {N}_{P P i} \cdot exp ( k t ) \qquad ( 19 )$$

The super BRC assay generates more photons compared to the standard BRC protocol discussed above. However, quantifying the original concentration of PPi involves kinetic analysis, in contrast to data analysis with normal BRC which analyzes steady state light emission.

In the super BRC method one or more primers may be designed to haye sequences specific to a target biomolecule of interest. The primers may be initially added into the solution where the target biomolecule is potentially present. If the target is present in the sample, the primer(s) anneals to the target DNA, and the quantity of the primed target DNA is equal to the number of original target molecules in the sample. If a polymerase enzyme is then added with dNTPs, the primed target DNA may be extended with incorporation of nucleotides by polymerization. A single PPi molecule is generated for each nucleotide incorporated. If the length of polymerization is known, the quantity of the target molecule can be quantified, and its concentration can be determined. The light intensity generated in this process is

$$I = α \cdot ( \frac{{k}_{L}}{V} ) \cdot {N}_{P} \cdot {L}_{X} \qquad ( 20 )$$

where N_{P} is the number of target biomolecules in the solution and Lx is the extendable length of the target nucleic acid and/or oligonucleotide probe.

Terminal Transferase Based Assays

Particular embodiments of the invention concern methods to detect, identify and/or quantify the presence of nucleic acids and/or other biomolecules linked to oligonucleotides and/or nucleic acids, by means of terminal transferase activity. Sources of and general methods applicable to terminal transferase assays are known in the art (e.g., Chang and Bollum, CRC Crit. Rev. Biochem., 21, 27-52, 1986; Roychoudhury et al., Nucl. Acids Res. 3, 101-116, 1976; Tu and Cohen, Gene 10, 177-183, 1980; Boule et al., J. Biol. Chem. 276, 31388-31393, 2001).

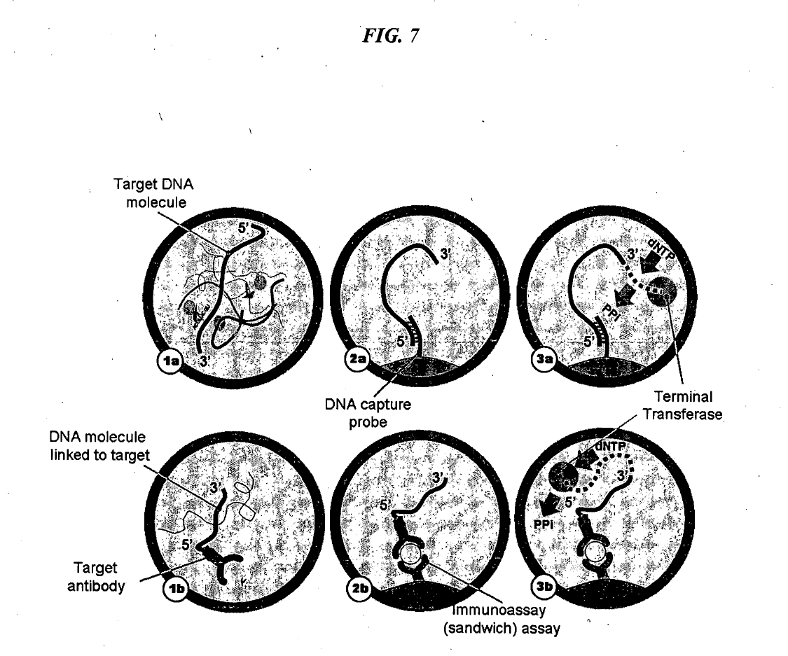

A general approach that may be used involves the initial capture or isolation of one or more specific DNA target molecules, or a target moiety containing DNA probes (e.g., antibody molecules linked with a DNA oligonucleotide) from the sample. Isolation can be carried out by various solid surface methods (e.g. capturing probe-coated magnetic beads), affinity matrices or electrophoretic processes. Once a target DNA has been captured or isolated, terminal transferase is added in the presence of nucleotides (dNTPs). Terminal transferase catalyzes the addition of dNTPs to the 3′ terminus of DNA. The enzyme works on single-stranded DNA (ssDNA), as well as the 3′ overhangs of double-stranded DNA (dsDNA). Its activity therefore resembles a DNA polymerase that does not require a primer, avoiding the need for a separate primer hybridization procedure. Because the enzyme can be used with double-stranded DNA, it does not require the separate isolation of single-stranded DNA. A general scheme for methods of use of terminal transferase for target molecule detection and/or quantitation is illustrated in FIG. 7.

As disclosed in FIG. 7, the target nucleic acid can either be free (1a-3a) or can be attached to another molecule, such as an antibody (1b-3b). In cases where the target is an RNA molecule, such as a messenger RNA (mRNA), the RNA may be converted to cDNA using reverse transcriptase, according to known protocols (e.g., Berger and Kimmel, 1987; Molecular Sambrook et al., 1989). The target nucleic acid may be captured, for example, by hybridization to a sequence specific capture probe (2a). Alternatively, target nucleic acids attached to another molecule may be captured by a variety of known immobilization methods, such as sandwich immunoassay (2b). Once captured, the substrate may be washed to remove unbound nucleic acids and the bound target may be extended using terminal transferase (3a, 3b). Where capture oligonucleotides are used, the 3′ end may be blocked, for example using dideoxy nucleotides, to prevent the terminal transferase from extending unhybridized capture probes.

The rate of terminal transferase mediated dNTP incorporation into the captured strand depends on the concentration of the enzyme, nucleotides and the relative amount of captured 3′ termini (which is in turn a function of the amount of target nucleic acid in the sample). Given the accurate determination of terminal transferase activity in a fixed time interval, and the initial nucleotide and enzyme concentrations, it is possible to correlate the measured terminal transferase activity with the concentration of target nucleic acid (total amount of 3′ terminus) in the sample.

Terminal transferase based assays measure the number of 3′ termini of DNA molecules in the sample, independent of the DNA being the actual target or just a reporter species linked to a secondary target. The enzyme can in theory incorporate unlimited number of nucleotides into the strand. However in a fixed time interval, depending on the activity of the enzyme, this number will be within a given deterministic range. A typical terminal transferase reaction may be performed, for example, at 20° C. in buffer containing 20 mM Tris acetate (pH 7.9) and 50 mM potassium acetate, supplemented with 1.5 mM CoCl₂. Alternative assay conditions include 50 nM potassium acetate, 20 mM Tris-acetate (pH 7.9), 10 mM magnesium acetate and 1 mM dithiothreitol, at 37° C. Additional conditions suitable for assay of terminal transferase activity are known (see, e.g., Chang and Bollum, 1986; Roychoudhury et al., 1976; Tu and Cohen, 1980; Boule et al. 2001).

Although a preferred substrate for terminal transferase is protruding 3′ ends, it will also less efficiently add nucleotides to blunt and 3′-recessed ends of ssDNA or dsDNA fragments. Cobalt is the necessary cofactor for activity of this enzyme. Terminal transferase may be purchased commercially (e.g., Fermentas, Inc., Hanover, Md.; Promega, Madison, Wis.; Stratagene, La Jolla, Calif.) and is usually produced by expression of the bovine gene in E. coli.

The growth of a DNA strand in a terminal transferase based assay can potentially result in a variety of detectable phenomena. Exemplary measurable changes produced by enzyme activity include, but are not limited to, intrinsic characteristics of the growing molecule itself (e.g., molecular mass, overall charge) as well as natural products of the incorporation reaction (e.g. PPi). Alternatively other effects can be measured using extrinsic modifications. These may include various labels or fluorogenic species attached to or incorporated into the nucleotide substrates. In preferred embodiments, the BRC assay system is used to detect PPi generated by terminal transferase activity.

Immuno-BRC Assays

In various embodiments of the invention, BRC assay methods may be utilized in combination with immunoassay techniques, to provide for highly sensitive and selective detection, identification and/or quantification of target antigens. Antibodies against target antigens may be commercially available or may be prepared as disclosed below. Antibody-based BRC assays may be of use in a variety of applications to analyze any target against which an antibody may be prepared.

In one exemplary embodiment of the invention, based on a sandwich ELISA type detection method, a primary antibody against a target molecule of interest may be attached to a surface. A sample suspected of containing the target molecule may be exposed to the surface to allow binding of the target to the primary antibody. After washing, a secondary antibody that binds to a different epitope of the same target molecule may be added. In various embodiments, the secondary antibody may be tagged with one or more oligonucleotides. In preferred embodiments, the secondary antibody may be labeled with a dextran molecule. Multiple oligonucleotide tags may be attached to dextran, allowing amplification of the BRC signal.

Dextran may be conjugated to antibodies by methods known in the art. For example, dextran-biotin conjugates may be purchased (e.g., Molecular Probes, Inc.) and attached to an avidin or streptavidin labeled antibody. Oligonucleotide tags may be prepared incorporating reactive groups for attachment to dextran, or may be purchased from commercial sources (e.g., amine-oligos, SH-oligos, acrydite-oligos or biotin-oligos from Integrated DNA Technologies, Coralville, Iowa). Methods for attachment of oligonucleotides to dextran may utilize published protocols (e.g., Gingeras et al., Nucleic Acids Res. 15:5373-90, 1987).

Tag oligonucleotides and/or nucleic acids bound to dextran may be used to detect secondary antibody binding to target molecules using any of the BRC techniques disclosed above, such as regular BRC, branched-chain BRC, transcription based BRC or super BRC. Alternatively, a terminal transferase-based BRC method may be used to detect, identify and/or quantify target biomolecules by immuno-BRC. In some embodiments of the invention, a self-priming oligonucleotide that hybridizes to itself may be used to initiate DNA polymerization and PPi generation for assay by BRC.

In alternative embodiments of the invention, luciferase may be attached to a primary or secondary antibody. Various immunoassay techniques, for example sandwich ELISA, may be performed to detect a target antigen. After binding and washing, reagents comprising ATP, APS, ATP sulfurylase and luciferin may be added to initiate bioluminescent detection.

The skilled artisan will realize that many variations on immuno-BRC methods may be utilized within the scope of the claimed methods. For example, in alternative embodiments a primary antibody may be directly labeled with tag oligonucleotides attached to dextran. Samples suspected of containing target biomolecules may be cross-linked to a solid surface and the primary antibody allowed to bind to the target for detection by BRC assay. In other alternatives, target molecules may be immobilized on a surface and reacted with an unlabeled primary antibody. A secondary antibody labeled with tag oligonucleotides attached to dextran may bind to the first antibody and be detected by BRC. The latter method offers the advantage that a single type of tagged secondary antibody (e.g., goat anti-mouse antibody) may be used to detect binding of a large number of primary antibodies.

In immuno-BRC assays where the tagged (secondary) antibody exhibits specific binding to a target molecule, a given sample may be assayed for a number of different target molecules either simultaneously or sequentially. For example, an antibody array may be prepared on a protein chip using standard methods. After exposure of a sample to the array, a mixture of secondary antibodies of differing specificities may be added to the chip. The presence of a target molecule is indicated by a signal (e.g., a bioluminescent signal) detected from a specific location on the chip. Using a sandwich immunoassay, detection of a target molecule on such a protein chip depends on the specificity of binding of both primary and secondary antibodies to the antigen. In other alternative embodiments, specificity of detection may depend upon the particular oligonucleotide tag attached to an antibody. A mixture of antibodies could be labeled each with a distinct oligonucleotide tag sequence. Upon binding of tagged antibodies to one or more target molecules, primers designed to hybridize to a single oligonucleotide tag sequence may be added sequentially, followed by addition of polymerase, nucleotides and BRC assay reagents. After generation of a signal, the tagged molecules could be washed, a new primer specific for a different oligonucleotide tag could be added and BRC detection performed again.

The skilled artisan will realize that many variations on known immunoassay techniques may be performed with BRC or other detection methods, and any such known immunoassay protocol may be utilized in the disclosed methods.

Thermostable Enzymes

In certain embodiments of the invention, the BRC assay and/or other detection methods may utilize thermostable enzymes, including but not limited to thermostable terminal transferase, polymerase, ATP sulfurylase and/or luciferase. Such thermostable enzymes may be of use for a variety of applications. Use of thermostable polymerases for thermal cycling processes, such as PCR, are well known in the art. In some embodiments, where detection of light emission or another type of signal occurs in real time, such thermal cycling processes may occur concurrently with BRC detection or other detection modalities. In such cases, thermostable detection enzymes such as luciferase and ATP sulfurylase may be utilized to avoid thermal inactivation during the PCR process. Alternatively, isothermal processes for nucleic acid and/or oligonucleotide amplification and/or detection may be conducted at elevated temperatures, utilizing thermostable enzymes. In certain embodiments, the use of thermostable enzymes would allow nucleic acid and/or oligonucleotide polymerization and detection to occur in a single step process, avoiding the need to separate the production of PPi or ATP from their detection.

Any thermostable enzyme known in the art may be utilized. Such enzymes are commercially available from a variety of sources, such as Taq polymerase (Roche Molecular Biochemicals, Indianapolis, Ind.), KlenTaq™ DNA Polymerase (Sigma-Aldrich, St. Louis, Mo.), Tgo DNA Polymerase (Roche Molecular Biochemicals), DyNAzyme™ DNA Polymerase (Finnzymes, Espoo, Finland) and GeneAmp® thermostable reverse transcriptase (Applied Biosystems, Foster City, Calif.). A thermostable form of luciferase (Ultraglow™ recombinant luciferase, Promega Corp., Madison, Wis., catalog #E140X) has been found by the inventors to be stable to about 95° C. Taq polymerase is a thermostable enzyme with terminal transferase activity.

A thermostable form of ATP sulfurylase has recently been reported (Hanna et al., Arch. Biochem. Biophys. 406:275-288, 2002). The open reading frame encoding the thermostable enzyme is available from GenBank (Accession No. AAC07134). Methods of preparation and purification of thermostable ATP sulfurylase are known (Hanna et al., 2002).

Apparatus for BRC Assays

To determine the quantity of PPi and/or ATP molecules present in BRC assays, the number of photons generated by the BRC process may be counted in selected time intervals, and the acquired waveform may be correlated to the target molecule characteristics and/or quantity. The generation of photons by luciferase in typical BRC assays has a quantum efficiency (Q.E.) of approximately 0.88 per consumed ATP molecule, and a maximum wavelength (depending on the type of luciferase) in the visible range of the electromagnetic spectrum (e.g. 565 nm for firefly luciferase).

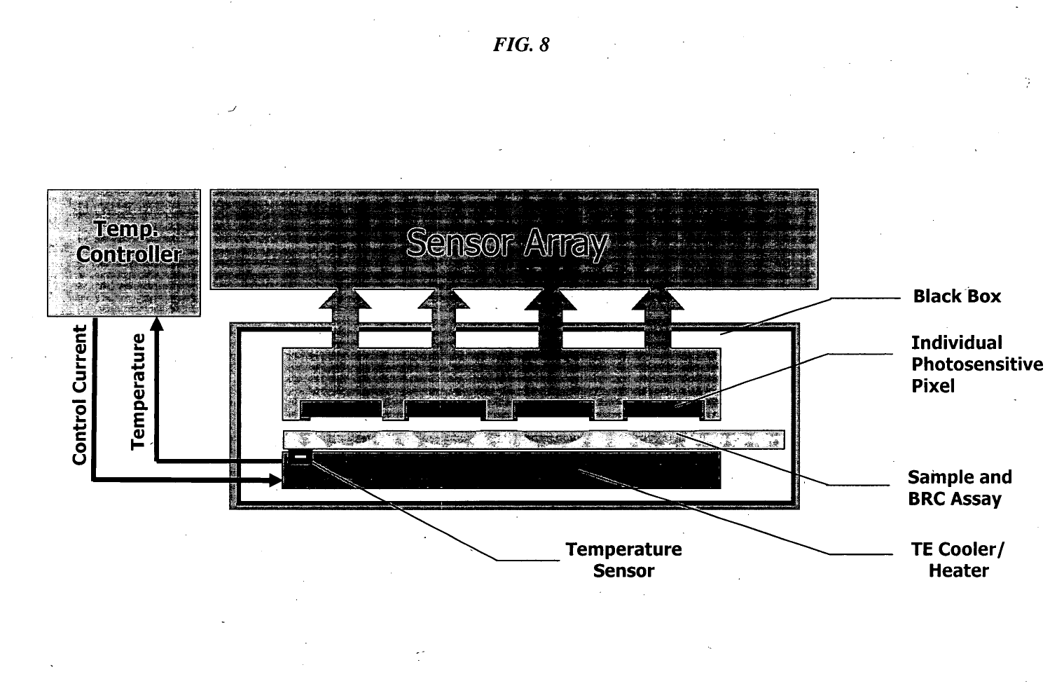

Establishing a controlled environment for the BRC assay facilitates reliable measurement of the photon generation rate and subsequent target molecule quantification. In certain preferred embodiments of the invention, the use of a reaction chamber with controllable temperature and minimum background light may be important for accurate target molecule quantification. In an exemplary embodiment illustrated in FIG. 8, an apparatus for BRC detection may comprise one or more of the following components.

i. Reaction chambers for BRC assay process, and/or affinity capture of targets

ii. Fluidic system to insert reagents or extract products from the reaction chambers

iii. Magnetic capturing devices

iv. Vibration generator and/or mixing device to increase convection

v. Optical coupling devices to convey the generated photons to a photodetector

vi. Photodetector to generate a relative photocurrent from the incident photons produced by BRC.

vii. Sensor array to efficiently acquire and measure photocurrent

viii. Cooling and/or heating device for controlling the reaction chamber temperature

ix. Cooling and/or heating device for controlling the photodetector and/or sensor temperature

x. Temperature controller module with a plurality of localized temperature sensors within the system to adjust the temperature based on user specifications.

xi. Data acquisition hardware to digitize the data from the sensor array

Reaction Chambers

In certain embodiments of the invention, a reaction chamber may contain reaction buffer, substrates, enzymes and reagents for the BRC or other detection assays. Alternatively, the reaction chamber may contain capture medium to allow target biomolecules (e.g. nucleic acids and/or proteins) to be specifically captured using different types of affinity matrices, functionalized gels and/or probes immobilized on solid surfaces (e.g. magnetic beads). Various methods of specific biomolecule capture, such as nucleic acid hybridization, antibody binding, aptamer binding, etc. are known in the art and any such known method may be used. Exemplary methods for preparing one or more binding moieties, such as antibodies or aptamers, for capture of target biomolecules are discussed in more detail below.

In particular embodiments of the invention, the binding moieties may be chemically attached to a hydrogel, such as a polyacrylamide based hydrogel (e.g., Yu et al., BioTechniques 34:1008-1022, 2003. Acrylamide monomers may be copolymerized with different probes (e.g., oligonucleotides, DNA, proteins, aptamers, etc.) by photoinduced polymerization of methacrylic modified monomers. Binding moieties may be localized in different reaction chambers. Alternatively, a single reaction chamber could potentially contain two or more hydrogels, each attached to a different binding moiety. The hydrogels may be attached to glass, silicone or other types of surfaces. Avidin-modified binding moieties may be attached to hydrogels containing biotin-modified monomers. Other methods of attaching binding moieties to hydrogels are known and may be used. The use of hydrogels improves the stability of binding moieties, such as proteins, and can maintain their binding activity for six months or longer (Yu et al., 2003). Hydrogel based chips may be utilized in combination with optical detection methods, such as BRC.

The binding moieties may be attached to the surface of the gel or alternatively may be embedded within the hydrogel to increase their stability. Where the binding moieties are embedded within the hydrogel, assays for the presence or absence of target molecules may also be performed within the gel. The hydrogel may be used to confine the reaction and/or enzymes, making localized BRC possible. Such assays may be performed using, for example, nucleic acid detection or immunoassay. The target and assay method are not limiting and virtually any target that can permeate into the hydrogel may be assayed by the disclosed method. Such localized assays allow for the possibility that more than one binding moiety-target interaction could be assayed within the same hydrogel.

The volume of the reaction chamber can vary anywhere between 1 nl (nanoliter) and 10 ml, but in most applications is typically between 2 μl (microliters) and 50 μl. In various embodiments, the reaction chamber may have an internal volume of about 1, 2, 5, 10, 20, 50, 100, 250, 500 or 750 nl, about 1, 2, 5, 10, 20, 50, 100, 250, 500 or 750 μl, or about 1, 2, 5 or 10 ml. The reaction chamber can comprise 96 well, 384 well, or other standard microtiter plates, and may be microfabricated by standard methods (e.g. etched, molded, drilled) in glass, silicon, ceramic, plastic, or composite materials. In preferred embodiments, the material used to construct the reaction chamber is optically transparent to allow detection of bioluminescence. The distance between chambers can vary from about 10 μm to about 5 cm, but in typical applications the distance will range between about 100 μm and about 1 cm. Each chamber may have a plurality of inlets and outlets, and may also be connected to other chambers by channels. In certain embodiments, different reactions and/or assay procedures may be performed sequentially in different chambers. For example, a first chamber may containing a target capturing matrix (e.g., oligonucleotide capture probe, aptamer, antibody) specific for a given target molecule. After capture of the specific target molecule, other target molecules that do not bind may be washed out and sent to a second chamber, which might contain other types of specific capturing matrices, resulting in a chamber-specific (site-specific) capture of different targets within a mono-directional or bi-directional flow-through system.

Fluidic Systems

A fluidic system may comprise components that facilitate the movement of solutions (e.g. reaction buffer) and/or gases (e.g. oxygen for luciferin oxidation) into and/or out of reaction chambers through specific inlets and/or outlets. The fluidic system typically comprises a plurality of pumps, fluidic channels, valves, and/or fluid reservoirs. The fluidic system is capable of delivering and/or extracting solutions or gases of volumes of about 1 pl to about 10 ml, but in typical applications the volume transferred at any given time will vary between about 1 nl to about 20 μl. The fluidic system may also be used to deliver and/or remove biological samples, magnetic particles, BRC or other reagents, primers, antibodies, etc. into the chambers.

Magnetic Capture Device

In particular embodiments of the invention, magnetic particles such as paramagnetic beads coated with capture (binding) moieties may be used to capture specific targets. In such embodiments, a magnetic field is generally induced to capture beads attached to target molecules and to wash out uncaptured species using a fluidic system. The magnetic field and/or fluidic system may also be used to move the beads to particular spatial locations at different points during the procedure. The magnetic field within the chambers and/or fluidic channels can be created by a plurality of independent permanent magnets, magnetic coils, and/or magnetic spiral inductors. In some embodiments of the invention the magnetic field intensity introduced on the field generator within the chamber may be modulated in order to release and capture the magnetic particles. In these cases, a permanent magnet can be mechanically placed in close proximity and/or into the designated chamber to capture beads, or moved away from the chamber to release beads. In the case of magnetic coils or spiral inductors, the release and capture of magnetic beads can be carried out by controlling the electrical current driving the coil, or spiral.

Magnetic particles, including magnetic particles derivatized for attachment of specific capture (binding) moieties, may be purchased, for example from Dynal Biotech (Dynabeads®, Lake Success, N.Y.). Alternatively, magnetic beads may be prepared by known methods (e.g., U.S. Pat. No. 4,267,234). Processes for the coupling of molecules to magnetic beads or a magnetite substrate are well known in the art (i.e. U.S. Pat. Nos. 4,695,393, 3,970,518, 4,230,685, and 4,677,055).

Vibration Generator and/or Mixing Device

In certain embodiments of the invention, mechanical motion may be used to stir, pump, filter, and/or manipulate gases, liquids, cells, bacteria, and other samples. In some applications electromechanical actuators and/or ultrasonic devices may be used to induce motion and/or create mechanical waves in the chamber and/or channels of the apparatus. Such devices may also affect the BRC or other reaction process. Electromechanical actuators are known in the art and may be purchased from standard sources.

Optical Coupling Device

Light that is generated from the reaction chambers, for example by BRC assay, may be collected and transferred to a photodetector using an optical coupling device. In alternative embodiments of the invention, the generated photons may either propagate for a short distance to a photodector, or may reach a photodetector substantially in contact with the chamber wall (distance from detector to chamber can vary from 1 μm to 1 m, but typically would range from 10 μm to 2 mm), or can be guided using an optical waveguide system (e.g. single optical fiber, or fiber bundle). In addition, different variations of lenses and/or mirrors may also be used to focus the generated light onto a photodetector device. An optical coupling device may also comprise one or more filters, which only pass certain wavelength regions relevant to the assay detection (e.g. 550 nm to 570 nm for Firefly luciferase photon emission). Optical fibers and other types of optical coupling devices are well known in the art and any such known device may be used in the disclosed apparatus.

Photodetector and Sensor Array

A number of different photosensitive devices can be used to measure the photon flux intensity from BRC or other optical assay. The devices can be photodiodes, avalanche photodiodes, phototransistors, vacuum photodiodes, silicon photodiodes, photomultiplier tubes (PMTs), multi anode photomultiplier tubes, charged-coupled devices (CCDs), CCD cameras, CMOS image sensors, photoresistive materials or any other optical detection device known in the art. The photodetector can be in a 2D array format, where an individual or plurality of sensors within the array measures the emitted light from a chamber selected from a plurality of reaction chambers. In certain embodiments a single photodetector can be used to sequentially measure light from multiple chambers, one (or several) at a time, in a sequential fashion. In some embodiments, the photodetector can be in close proximity to the chamber and/or even integrated onto the chambers. As an example one could use an array of photodiodes in silicon wafers, where chambers are etched into either the oxide top layers, or the bulk silicon wafer. As another example a micro-fluidic chip can be used, where the reaction chambers are connected via micro-channels and the whole chip is put onto the surface of a semiconductor based image sensor (e.g. CMOS or CCD), where the light from each well directly impinges on a photosensitive section of the imager.

In certain embodiments of the invention, a highly sensitive cooled CCD detector may be used. The cooled CCD detector has a probability of single-photon detection of up to 80%, a high spatial resolution pixel size (5 microns), and sensitivity in the visible through near infrared spectra. (Sheppard, Confocal Microscopy: Basic Principles and System Performance in: Multidimensional Microscopy, P. C. Cheng et al. eds., Springer-Verlag, New York, N.Y. pp. 1-51, 1994.) In another embodiment of the invention, a coiled image-intensified coupling device (ICCD) may be used as a photodetector that approaches single-photon counting levels (U.S. Pat. No. 6,147,198). A small number of photons triggers an avalanche of electrons that impinge on a phosphor screen, producing an illuminated image. This phosphor image is sensed by a CCD chip region attached to an amplifier through a fiber optic coupler.

In some embodiments of the invention, an avalanche photodiode (APD) may be made to detect low light levels. The APD process uses photodiode arrays for electron multiplication effects (U.S. Pat. No. 6,197,503). The invention is not limited to the disclosed embodiments and it is contemplated that any light detector known in the art that is capable of accumulating photons over a time interval may be used in the disclosed methods and apparatus.

The output of the photodetector is typically in form of a photocurrent and/or voltage, which has a relationship to the incident photon flux to the detector. The output of the sensor depends on the topology, number of photodetector elements and characteristics of individual photodetectors, and may be in parallel (i.e. all output channels are on separate lines), or sequential (i.e. one output is connected to the output line at a time).

Temperature Control Devices

In some embodiments of the invention, the reaction chambers and/or photosensors are designed to be temperature controlled, for example by incorporation of Peltier elements or other methods known in the art. Methods of controlling temperature for low volume liquids used in nucleic acid polymerization or other reactions are known in the art. (See, e.g., U.S. Pat. Nos. 5,038,853, 5,919,622, 6,054,263 and 6,180,372.) Methods for maintaining temperature control of sensing elements are also known in the art.

In certain embodiments of the invention, cyclic changes in temperature in one or more reaction chambers (e.g., as used in the PCR process) may be useful. The temperature profile can vary from 0° C. and 100° C., but in most BRC applications varies between room temperature and 95° C. Each chamber may be individually thermally controlled, for example with a different heater/cooler device and a temperature sensor (e.g. thermocouple, or a thermistor) associated with each chamber. Alternatively, a plurality of chambers may be commonly thermally controlled, using a single temperature controller and sensor.

Different types of known heating and/or cooling devices may be used, such as resistive heaters, Peltier devices, heat sinks, fluidic cooling and heating devices and laser cooling and heating. As an example, Peltier devices, also known as thermoelectric (TE) modules, are small solid-state devices that function as heat pumps, transferring heat from one location to another. Peltier devices may be incorporated into an apparatus in contact with the reaction chambers to form a temperature-contolled reaction chamber unit. A typical Peltier unit is a few millimeters thick by a few millimeters to a few centimeters square. It is a sandwich formed by two ceramic plates with an array of small bismuth telluride cubes (“couples”) in between. When a direct current (DC) is applied, heat is pumped from one side of the unit to the other, at which point the heat can be removed with a heat sink or other cooling means. Heat may be pumped in either direction, allowing alternate heating or cooling of the chamber.

A heating or cooling module may also be used to control the temperature of the photosensors (e.g. photodiodes) used in the system. The performance of photodiodes, for example, is extremely dependent upon temperature. Temperature can affect both the quantum efficiency and even more dramatically the dark current and therefore the noise characteristics of such photosensors. In preferred embodiments of the invention, the sensors will have a fixed temperature during measurements of light emission. A cooling/heating device may be either integrated with, or put into contact with, the photosensor in order to maintain a predetermined temperature or a time-dependent temperature cycle.

Thermal Controller

The heating and cooling devices may be individually controlled by a controller means. In the case of TE cooling, an electronic controller module may sense the temperature of each designated heating/cooling location. Based on the difference between the actual and predetermined temperature, the controller pumps heat into or out of the location until the location temperature reaches its predetermined (null point) value. The controller means in turn may be controlled by a computer or similar secondary controller device, having a user interface so that a predetermined temperature, or predetermined series of temperatures, or predetermined cycles of a repeated temperature series may be selected by the user. Such computer systems and temperature controller means are well known in the art and can incorporate any of a wide variety of temperature-control devices well known to those skilled in the art.

Certain embodiments of the invention concern a portable, ultra-sensitive, pathogen detection system that can identify predetermined pathogens and, their antibiotic resistance profiles in biological samples. Applications of this device include detection and quantification of the presence of predetermined microbe species in air or sterile biological fluids such as blood, cerebrospinal fluid and urine. Detection can be performed in a much more rapid and accurate fashion than is currently possible. Such systems may comprise an apparatus as disclosed herein, designed for use as a microfluidic system.

Microfluidic Structures

In various embodiments of the invention, microfluidic devices may be used to provide samples to specific capture sites and to process such samples for target molecule detection. The small volumes of microfluidic devices allow processing of small sample volumes. Given the small detection volumes for BRC assays, background luminescence from the system will also be low. The combination of a low sample volume and low background luminescence allows for particularly high sensitivity of detection. Microfluidic devices comprise one or more channels of micron-size depth and width, generally between 10 and 900 microns. The channels may be of varying length but generally are between 0.1 and 100 cm in length. Microfluidic devices therefore contain very small volumes defined by each channel, generally ranging from 100 picoliters to 100 microliters. Because of their small internal volumes, reagent consumption is low, only a few target biomolecules are required to create a measurable signal, the devices are compact and easily stored and transported, and the devices may be designed to be disposable and convenient to use.

Low reagent consumption is especially important when expensive or difficult to obtain reagents are used. When used, for example, for pathogen detection, the number of microorganisms required to be detected can be very low, allowing detection limits for example of a single cell, 2 or more cells, 10 cells, 100 cells or 1000 cells. The microfluidic channels may be formed from any substance having a surface compatible with biological materials. In exemplary embodiments of the invention, the channels (or at least the surface of the channels) may be made of glass, fused silica, quartz or silicon. (See, e.g, Bousse et al., “Electrokinetically Controlled Microfluidic Analysis Systems,” Ann. Rev. Biophys. Biomol. Struct. 29:155-181, 2000.)

Other materials that may be used for construction of microfluidic devices include organic polymers (i.e. plastics) such as methacrylates, polystyrene, polypropylene, polycarbonate, polyethylene, or the like. Soft polymeric materials such as organosilanes, including polydimethylsilane (PDMS) can be used to fabricate the microfluidic channels. The soft polymers alternatively may be polyacrylamide materials or mixed polymers containing co-polymerized organic or inorganic substances. An advantage of soft polymers is that they are deformable by applying external pressure. Application of external pressure results in creation of a closed valve. Because the soft polymer materials can be elastic, release of the pressure results in reopening of the valve. Flow in the channel is restored provided that a gradient in pressure is created along the length of the channel. (See, e.g., Thorsen et al., “Microfluidic Large-Scale Integration,” Science 298:580-586, 2002.) Application of external pressure adjacent to a closed valve creates pressure that may be used to pump fluids. Alternatively, the pressure may be created by application of gas pressure, application of a vacuum (relative to ambient pressure) or by applying an electrical field along the channel and creating a pressure gradient by electroendosmosis. All of these processes are well known in the art.

Micro-Electro-Mechanical Systems (MEMS)

In some embodiments of the invention, the chambers, sensors and other components of the disclosed apparatus may be incorporated into one or more Micro-Electro-Mechanical Systems (MEMS). MEMS are integrated systems that may comprise mechanical elements, actuator elements, control elements, detector elements and/or electronic elements. All of the components may be manufactured by known microfabrication techniques on a common chip, comprising a silicon-based or equivalent substrate (e.g., Voldman et al., Ann. Rev. Biomed. Eng. 1:401-425, 1999).

The electronic components of MEMS may be fabricated using integrated circuit (IC) processes (e.g., CMOS, Bipolar, or BICMOS processes). They may be patterned using photolithographic and etching methods known for semiconductor chip manufacture. The micromechanical components may be fabricated using “micromachining” processes that selectively etch away parts of the silicon wafer and/or add new structural layers to form the mechanical and/or electromechanical components. Basic techniques in MEMS manufacture include depositing thin films of material on a substrate, applying a patterned mask on top of the films by photolithographic imaging or other known lithographic methods, and selectively etching the films. A thin film may have a thickness in the range of a few nanometers to 100 micrometers. Deposition techniques of use may include chemical procedures such as chemical vapor deposition (CVD), electrodeposition, epitaxy and thermal oxidation and physical procedures like physical vapor deposition (PVD) and casting. Sensor layers of 5 nm thickness or less may be formed by such known techniques. Standard lithography techniques may be used to create sensor layers of micron or sub-micron dimensions, operably coupled to detectors.

The manufacturing method is not limiting and any methods known in the art may be used, such as atomic layer deposition, pulsed DC magnetron sputtering, vacuum evaporation, laser ablation, injection molding, molecular beam epitaxy, dip-pen nanolithograpy, reactive-ion beam etching, chemically assisted ion beam etching, microwave assisted plasma etching, focused ion beam milling, electron beam or focused ion beam technology or imprinting techniques. Methods for manufacture of nanoelectromechanical systems may be used for certain embodiments of the invention. (See, e.g., Craighead, Science 290:1532-36,0.)

In some embodiments, the reaction chamber and other components of the apparatus may be manufactured as a single integrated chip. Such a chip may be manufactured by methods known in the art, such as by photolithography and etching. However, the manufacturing method is not limiting and other methods known in the art may be used, such as laser ablation, injection molding, casting, or imprinting techniques. Microfabricated chips are commercially available from sources such as Caliper Technologies Inc. (Mountain View, Calif.) and ACLARA BioSciences Inc. (Mountain View, Calif.).

In a non-limiting example, Borofloat glass wafers (Precision Glass & Optics, Santa Ana, Calif.) may be pre-etched for a short period in concentrated HF (hydrofluoric acid) and cleaned before deposition of an amorphous silicon sacrificial layer in a plasma-enhanced chemical vapor deposition (PECVD) system (PEII-A, Technics West, San Jose, Calif.). Wafers may be primed with hexamethyldisilazane (HMDS), spin-coated with photoresist (Shipley 1818, Marlborough, Mass.) and soft-baked. A contact mask aligner (Quintel Corp. San Jose, Calif.) may be used to expose the photoresist layer with one or more mask designs, and the exposed photoresist removed using a mixture of Microposit developer concentrate (Shipley) and water. Developed wafers may be hard-baked and the exposed amorphous silicon removed using CF₄ (carbon tetrafluoride) plasma in a PECVD reactor. Wafers may be chemically etched with concentrated HF to produce the reaction chamber and any channels. The remaining photoresist may be stripped and the amorphous silicon removed.

Access holes may be drilled into the etched wafers with a diamond drill bit (Crystalite, Westerville, Ohio). A finished chip may be prepared by thermally bonding an etched and drilled plate to a flat wafer of the same size in a programmable vacuum furnace (Centurion VPM, J. M. Ney, Yucaipa, Calif.). In certain embodiments, the chip may be prepared by bonding two etched plates to each other. Alternative exemplary methods for fabrication of a reaction chamber chip are disclosed in U.S. Pat. Nos. 5,867,266 and 6,214,246.

Nucleic Acids and Oligonucleotides

In various embodiments of the invention, samples comprising nucleic acids may be prepared by any technique known in the art. In certain embodiments, analysis may be performed on crude sample extracts, containing complex mixtures of nucleic acids, proteins, lipids, polysaccharides and other compounds. Such samples are likely to contain contaminants that could potentially interfere with the BRC process or other detection methods. In preferred embodiments, nucleic acids may be partially or fully separated from other sample constituents before analysis to detect target nucleic acids.

Methods for partially or fully purifying DNA and/or RNA from complex mixtures, such as cell homogenates or extracts, are well known in the art. (See, e.g., Guide to Molecular Cloning Techniques, eds. Berger and Kimmel, Academic Press, New York, N.Y., 1987; Molecular Cloning: A Laboratory Manual, 2nd Ed., eds. Sambrook, Fritsch and Maniatis, Cold Spring Harbor Press, Cold Spring Harbor, N.Y., 1989). Generally, cells, tissues or other source material containing nucleic acids are first homogenized, for example by freezing in liquid nitrogen followed by grinding in a mortar and pestle. Certain tissues may be homogenized using a Waring blender, Virtis homogenizer, Dounce homogenizer or other homogenizer. Crude homogenates may be extracted with detergents, such as sodium dodecyl sulphate (SDS), Triton X-100, CHAPS (3-[(3-cholamidopropyl)-dimethylammonio]-1-propane sulfonate), octylglucoside or other detergents known in the art. As is well known, nuclease inhibitors such as RNase or DNase inhibitors may be added to prevent degradation of target nucleic acids.

Extraction may also be performed with chaotrophic agents such as guanidinium isothiocyanate, or organic solvents such as phenol. In some embodiments, protease treatment, for example with proteinase K, may be used to degrade cell proteins. Particulate contaminants may be removed by centrifugation or ultracentrifugation. Dialysis against aqueous buffer of low ionic strength may be of use to remove salts or other soluble contaminants. Nucleic acids may be precipitated by addition of ethanol at −20° C., or by addition of sodium acetate (pH 6.5, about 0.3 M) and 0.8 volumes of 2-propanol. Precipitated nucleic acids may be collected by centrifugation or, for chromosomal DNA, by spooling the precipitated DNA on a glass pipet or other probe. The skilled artisan will realize that the procedures listed above are exemplary only and that many variations may be used, depending on the particular type of nucleic acid to be analyzed.

In certain embodiments, nucleic acids to be analyzed may be naturally occurring DNA or RNA molecules. Virtually any naturally occurring nucleic acid may be analyzed by the disclosed methods including, without limit, chromosomal, mitochondrial or chloroplast DNA or ribosomal, transfer, heterogeneous nuclear or messenger RNA. Nucleic acids may be obtained from either prokaryotic or eukaryotic sources by standard methods known in the art. Alternatively, nucleic acids of interest may be prepared artificially, for example by PCR™ or other known amplification processes or by preparation of libraries such as BAC, YAC, cosmid, plasmid or phage libraries containing nucleic acid inserts. (See, e.g., Berger and Kimmel, 1987; Sambrook et al., 1989.) The source of the nucleic acid is unimportant for purposes of analysis and it is contemplated within the scope of the invention that nucleic acids from virtually any source may be analyzed.

Nucleic Acid Replication

In certain embodiments of the invention, target nucleic acids may be amplified and/or replicated prior to or during detection. Amplification may be accomplished by any technique known in the art. Exemplary embodiments are disclosed below.

Primers

The term primer, as used herein, is meant to encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-stranded or single-stranded form, although the single-stranded form is preferred. Primers may be prepared, for example, using oligonucleotide synthesizers available from standard commercial sources (e.g., Applied Biosystems, Foster City, Calif.). Alternatively, primers of any selected sequence may be obtained from standard commercial sources (e.g., Midland Certified Reagents, Midland, Tex.). Such commercial primers may be purchased with specific chemical modifications, for example, attachment of a biotin moiety or other reactive group to facilitate immobilization of the primer to a solid surface or attachment of a label or other group. In certain embodiments of the invention, primers incorporating a preexisting label moiety may be purchased from commercial sources. Methods for selection, design and validation of primer sequences to amplify any given target nucleic acid and/or oligonucleotide tag sequence are well known in the art.

Polymerases

In certain embodiments of the invention, the disclosed methods may involve binding of a DNA polymerase to a primer molecule and the catalyzed addition of nucleotide precursors to the 3′ end of a primer. In alternative embodiments, other types of polymerase, such as RNA polymerase, may be utilized that do not require primers but rather bind to promoter sequences to initiate RNA polymerization. Non-limiting examples of polymerases of potential use include DNA polymerases, RNA polymerases, reverse transcriptases, and RNA-dependent RNA polymerases. The differences between these polymerases in terms of their requirement or lack of requirement for primers or promoter sequences are known in the art.

Non-limiting examples of polymerases that may be of use include Thermatoga maritima DNA polymerase, AmplitaqFS™ DNA polymerase, Taquenase™ DNA polymerase, ThermoSequenase, Taq DNA polymerase, Qbeta™ replicase, T4 DNA polymerase, Thermus thermophilus DNA polymerase, RNA-dependent RNA polymerase and SP6 RNA polymerase. Commercially available polymerases including Pwo DNA Polymerase from Boehringer Mannheim Biochemicals (Indianapolis, Ind.); Bst Polymerase from Bio-Rad Laboratories (Hercules, Calif.); IsoTherm™ DNA Polymerase from Epicentre Technologies (Madison, Wis.); Moloney Murine Leukemia Virus Reverse Transcriptase, Pfu DNA Polymerase, Avian Myeloblastosis Virus Reverse Transcriptase, Thermus flavus (Tfl) DNA Polymerase and Thermococcus litoralis (Tli) DNA Polymerase from Promega (Madison, Wis.); RAV2 Reverse Transcriptase, HIV-1 Reverse Transcriptase, T7 RNA Polymerase, T3 RNA Polymerase, SP6. RNA Polymerase, RNA Polymerase E. coli, Thermus aquaticus DNA Polymerase, T7 DNA Polymerase +/−3′→5′ exonuclease, Klenow Fragment of DNA Polymerase I, Thermus ‘ubiquitous’ DNA Polymerase, and DNA polymerase I from Amersham Pharmacia Biotech (Piscataway, N.J.).

As is known in the art, various polymerases have an endogenous 3′-5′ exonuclease activity that may be used for proof-reading newly incorporated nucleotides. Because a molecule of pyrophosphate is generated for each nucleotide incorporated into a growing chain, regardless of whether or not it is subsequently removed, in certain embodiments of the invention it may be preferred to use polymerases that are lacking exonuclease or proof-reading activity. Methods of using polymerases and compositions suitable for use in such methods are well known in the art (e.g., Berger and Kimmel, 1987; Sambrook et al., 1989).

Amplification Methods

A number of template dependent processes are available to amplify target nucleic acids. One of the best known amplification methods is the polymerase chain reaction (referred to as PCR™) which is described in U.S. Pat. Nos. 4,683,195, 4,683,202 and 4,800,159, and in Innis et al. (PCR Protocols, Academic Press, Inc., San Diego Calif., 1990).

Briefly, in PCR, two primer sequences are prepared which are complementary to regions on opposite complementary strands of, for example, a target nucleic acid. An excess of deoxynucleoside triphosphates are added to a reaction mixture along with a DNA polymerase, e.g., Taq polymerase. If the target sequence is present in a sample, the primers will bind to the target and the polymerase will cause the primers to be extended along the target sequence by adding on nucleotides. By raising and lowering the temperature of the reaction mixture, the extended primers will dissociate from the nucleic acid to form reaction products, excess primers will bind to the nucleic acid and to the reaction products and the process is repeated.

A reverse transcriptase PCR amplification procedure may be performed in order to amplify mRNA. Methods of reverse transcribing RNA into cDNA are well known and disclosed, for example, in Sambrook et al. (1989). Alternative methods for reverse transcription utilize thermostable DNA polymerases. These methods are disclosed in WO 0.90/07641 filed Dec. 21, 1990. Polymerase chain reaction methodologies are well known in the art.

Qbeta Replicase, disclosed in PCT Application No. PCT/US87/00880, may also be used for amplification. In this method, a replicative sequence of RNA which has a region complementary to that of a target nucleic acid is added to a sample in the presence of an RNA polymerase. The polymerase will copy the replicative sequence which may then be detected.

Strand Displacement Amplification (SDA) is an isothermal method of carrying out amplification of target nucleic acids that involves multiple rounds of strand displacement and synthesis, i.e., nick translation. A similar method, called Repair Chain Reaction (RCR), involves annealing several probes throughout a region targeted for amplification, followed by a repair reaction.

Still other amplification methods are disclosed in GB Application No. 2 202 328, in which “modified” primers are used in a PCR like process. The primers may be modified by labeling with a capture moiety (e.g., biotin) and/or a detector moiety (e.g., enzyme). Other nucleic acid amplification procedures include transcription-based amplification systems (TAS), nucleic acid sequence based amplification (NASBA) and 3SR. (See, Kwoh et al., Proc. Nat. Acad. Sci. USA, 86: 1173, 1989) and PCT Application WO 88/10315.) These amplification techniques involve annealing a primer which has target nucleic acid specific sequences. Following polymerization, DNA/RNA hybrids are digested with RNase H while double stranded DNA molecules are heat denatured again. In either case the single stranded DNA is made fully double stranded by addition of second target nucleic acid specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once again with a polymerase such as T7 or SP6.

Davey et al., European Application No. 329 822 disclose a nucleic acid amplification process involving cyclically synthesizing single-stranded RNA (“ssRNA”), ssDNA, and double-stranded DNA (dsDNA). The ssRNA is a first template for a first primer oligonucleotide, which is elongated by reverse transcriptase. The RNA is then removed from the resulting DNA:RNA duplex by the action of ribonuclease H. The resultant ssDNA is a second template for a second primer, which also includes the sequences of an RNA polymerase promoter (exemplified by T7 RNA polymerase) 5′ to its homology to the template. This primer is then extended by DNA polymerase, resulting in a double-stranded DNA (“dsDNA”) molecule having a sequence identical to that of the original RNA between the primers and having additionally, at one end, a promoter sequence. This promoter sequence may be used by the appropriate RNA polymerase to make many RNA copies of the DNA. These copies may then re-enter the cycle, leading to very swift amplification. With proper choice of enzymes, this amplification may be done isothermally without addition of enzymes at each cycle. Because of the cyclical nature of this process, the starting sequence may be chosen to be in the form of either DNA or RNA.

Miller et al., PCT Application WO 89/06700 disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods including “race” and “one-sided PCR” are known in the art and any such known method may be used. (See, e.g., Frohman, In: PCR™ Protocols: A Guide To Methods And Applications, Academic Press, N.Y., 1990; Ohara et al., Proc. Nat'l Acad. Sci. USA, 86:5673-5677, 1989).

Kurn et al. (U.S. Pat. No. 6,251,639) disclose an isothermal, single primer linear nucleic acid amplification method. In this approach, methods for amplifying complementary DNA using a composite primer, primer extension, strand displacement, and optionally a termination sequence, are provided, as well as methods for amplifying sense RNA using a composite primer, primer extension, strand displacement, optionally template switching, a propromoter oligonucleotide and transcription.

Promoters

In various embodiments of the invention involving transcription of a DNA strand by an RNA polymerase, it may be desirable to incorporate a promoter sequence, for example into a primer. A “promoter” refers to a DNA sequence recognized by an RNA polymerase to initiate transcription. Depending on the application, a promoter may be a eukaryotic promoter or a prokaryotic promoter, to be used respectively with eukaryotic or prokaryotic RNA polymerases. Promoter elements recognized by eukaryotic and prokaryotic RNA polymerases are known in the art and any such known elements may be used.

The term promoter refers generically to a group of transcriptional control modules that are clustered around the initiation site for RNA polymerase. Promoters are composed of discrete functional modules, each consisting of approximately 7-20 bp of DNA, and containing one or more recognition sites for transcriptional activator or repressor proteins. At least one module in each promoter functions to position the start site for RNA synthesis. The best known example of this is the TATA box (or Pribnow box in prokaryotes), but in some promoters lacking a TATA box, a discrete element overlying the start site helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another.

The particular promoter that is employed to initiate transcription is not believed to be important. In various embodiments, the human cytomegalovirus (CMV) immediate early gene promoter, the SV40 early promoter or the Rous sarcoma virus long terminal repeat can be used to obtain high-level transcription by eukaryotic RNA Polymerase II. The use of other viral, mammalian or bacterial promoters which are well-known in the art is also contemplated. Any promoter/enhancer combination (e.g., Eukaryotic Promoter Data Base) could be used to drive transcription of a target nucleic acid sequence.

Tables 2 and 3 list various eukaryotic enhancers/promoters that may be employed to regulate transcription. Enhancers are genetic elements that increase transcription from a eukaryotic promoter located at a distant position on the same molecule of DNA. Enhancers are organized much like promoters. That is, they are composed of many individual elements, each of which binds to one or more transcriptional proteins. The skilled artisan will recognize that in addition to the listed promoters/enhancers, many prokaryotic promoters are known and may be used to drive transcription. Such prokaryotic promoter sequences include, but are not limited to, the lac promoter, the B-gal promoter, the lambda promoter, the fd promoter, the trp promoter, the T7 promoter, etc. Many prokaryotic promoters are commercially available from standard sources. Inducible promoter elements are disclosed in Table 3. In some embodiments of the invention, it may be preferable to activate transcription at specific points in the procedure. In such case, use of an inducible promoter allows precise control of the timing of RNA polymerase activity.

TABLE 2
ENHANCER/PROMOTER

Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Prealbumin (Transthyretin)
Muscle Creatine Kinase
Elastase I
Metallothionein
Collagenase
Albumin Gene
α-Fetoprotein
τ-Globin
β-Globin
e-fos
c-HA-ras
Insulin
Neural Cell Adhesion Molecule (NCAM)
α1-Antitrypsin
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus

TABLE 3

| Element | Inducer |
|---|---|
| MT II | Phorbol Ester (TPA) |
| | Heavy metals |
| MMTV (mouse mammary tumor | Glucocorticoids |
| virus) | |
| β-Interferon | poly(rI)X, poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone α | Thyroid Hormone |
| Gene | |
| Insulin E Box | Glucose |

Proteins, Polypeptides and Peptides

In various embodiments of the invention, the target biomolecule(s) of interest may comprise one or more proteins, polypeptides or peptides. These terms are used interchangeably herein. In different embodiments, proteins to be analyzed may be purified from natural sources, expressed by in vitro translation of an mRNA species or by linked transcription/translation of a DNA species, and/or expressed in a host cell that has been transformed with a gene or a complementary DNA (cDNA) species. These methods are not limiting and proteins to be analyzed may be prepared by any method known in the art.

Protein Purification

In certain embodiments of the invention, proteins to be analyzed may be partially or fully purified from a variety of sources before analysis. Protein purification techniques are well known in the art. These techniques typically involve an initial fractionation of cell or tissue homogenates and/or extracts into protein and non-protein fractions. Fractionation may utilize, for example, differential solubility in aqueous solutions, detergents and/or organic solvents, elimination of classes of contaminants such as nucleic acids by enzymatic digestion, precipitation of proteins with ammonium sulphate, polyethylene glycol, antibodies, heat denaturation and the like, followed by ultracentrifugation. Low molecular weight contaminants may be removed by dialysis, filtration and/or organic phase extraction.

Protein(s) of interest may be purified using chromatographic and/or electrophoretic techniques to achieve partial or complete purification. Methods suited to the purification of proteins include, but are not limited to, ion-exchange chromatography, gel exclusion chromatography, polyacrylamide gel electrophoresis, affinity chromatography, immunoaffinity chromatography, hydroxylapatite chromatography, hydrophobic interaction chromatography, reverse phase chromatography, isoelectric focusing, fast protein liquid chromatography (FPLC) and high pressure liquid chromatography (HPLC). These and other methods of protein purification are known in the art and are not limiting for the claimed subject matter. There is no requirement that the protein must be in its most purified state and methods exhibiting a lower degree of relative purification may, for example, have advantages in increased recovery of target protein.

Particular embodiments of the invention may rely on affinity chromatography for purification and/or immobilization of proteins. The method relies on an affinity between a protein and a molecule to which it can specifically bind. Chromatography material may be prepared by covalently attaching a protein-binding ligand, such as an antibody, antibody fragment, receptor protein, substrate, inhibitor, product or an analog of such ligands to an insoluble matrix, such as column chromatography beads, magnetic beads or a nylon or other membrane. The matrix is then able to specifically adsorb the target protein from a solution. Elution occurs by changing the solvent conditions (e.g. pH, ionic strength, temperature, detergent concentration, etc.). One of the most common forms of affinity chromatography is immunoaffinity chromatography. Methods for generating antibodies against various types of proteins for use in immunoaffinity chromatography are well known in the art, discussed in more detail below.

In some embodiments of the invention, one or more proteins of interest may be specifically labeled. Various methods for protein labeling are known in the art, discussed in more detail below.

In Vitro Translation

Proteins may be expressed using an in vitro translation system with mRNA templates. Complete kits for performing in vitro translation are available from commercial sources, such as Ambion (Austin, Tex.), Promega (Madison, Wis.), Invitrogen (Carlsbad, Calif.) and Novagen (Madison, Wis.). Such kits may utilize total RNA, purified polyadenylated mRNA, and/or purified individual mRNA species obtained from a cell, tissue or other sample. Methods of preparing different RNA fractions and/or individual mRNA species for use in in vitro translation are known. (E.g., Sambrook, et al., 1989; Ausubel et al., Current Protocols in Molecular Biology, Wiley and Sons, New York, N.Y., 1994).

In certain alternative embodiments of the invention, in vitro translation may be linked to transcription of genes to generate mRNAs. Such linked transcription/translation systems may use PCR amplification products and/or DNA sequences inserted into standard expression vectors such as BACs (bacterial artificial chromosomes), YACs (yeast artificial chromosomes), cosmids, plasmids, phage and/or other known expression vectors. Linked transcription/translation systems are available from commercial sources (e.g., Proteinscript™ II kit, Ambion, Austin, Tex.; Quick Coupled System, Promega, Madison, Wis.; Expressway, Invitrogen, Carlsbad, Calif.). Such systems may incorporate various elements to optimize the efficiency of transcription and translation, such as polyadenylation sequences, consensus ribosomal binding (Kozak) sequences, Shine-Dalgarno sequences and/or other regulatory sequences known in the art.

Protein Expression in Host Cells

Nucleic acids encoding target proteins of interest may be incorporated into expression vectors for transformation into host cells and production of the encoded proteins. Non-limiting examples of host cell lines known in the art include bacteria such as E. coli, yeast such as Pichia pastoris, and mammalian cell lines such as VERO cells, HeLa cells, Chinese hamster ovary cell lines, human embryonic kidney (HEK) 293 cells, mouse neuroblastoma N2A cells, or the W138, BHK, COS-1, COS-7, 293, HepG2, 3T3, RIN, L-929 and MDCK cell lines. These and other host cell lines may be obtained from standard sources, such as the American Type Culture Collection (Rockville, Md.) or commercial vendors.

A complete gene can be expressed or fragments of a gene encoding portions of a protein can be expressed. The gene or gene fragment encoding protein(s) of interest may; be inserted into an expression vector by standard cloning techniques. Expression libraries containing part or all of the messenger RNAs expressed in a given cell or tissue type may be prepared by known techniques or commercially purchased. Such libraries may be screened for clones encoding particular proteins of interest, for example using antibody or oligonucleotide probes and known screening techniques.

The engineering of DNA segment(s) for expression in a prokaryotic or eukaryotic system may be performed by techniques generally known in the art. Any known expression system may be employed for protein expression. Expression vectors may comprise various known regulatory elements for protein expression, such as promoters, enhancers, ribosome binding sites, termination sequences, polyadenylation sites, etc.

Promoters commonly used in bacterial expression vectors include the β-lactamase, lactose and tryptophan promoter systems. Suitable promoter sequences in yeast expression vectors include the promoters for 3-phosphoglycerate kinase or other glycolytic enzymes. Promoters of use for mammalian cell expression may be derived from the genome of mammalian cells (e.g., metallothionein promoter) or from mammalian viruses (e.g., the adenovirus late promoter or the early and late promoters of SV40). Many other promoters are known and may be used in the practice of the disclosed methods.

Eukaryotic expression systems of use include, but are not limited to, insect cell systems infected with, for example, recombinant baculovirus, or plant cell systems infected with recombinant cauliflower mosaic virus or tobacco mosaic virus. In an exemplary insect cell system, Autographa californica nuclear polyhidrosis virus is used as a vector to express foreign genes in Spodoptera frugiperda cells or the Hi5 cell line (Invitrogen, Carlsbad, Calif.). Nucleic acid coding sequences are cloned into, for example, the polyhedrin gene of the virus under control of the polyhedrin promoter. Recombinant viruses containing the cloned gene are then used to infect Spodoptera frugiperda cells and the inserted gene is expressed (e.g., U.S. Pat. No. 4,215,051; Kitts et al., Biotechniques 14:810-817, 1993; Lucklow et al., J. Virol., 67:4566-79, 1993). Other exemplary insect cell expression vectors are based on baculovirus vectors, for example, pBlueBac (Invitrogen, Sorrento, Calif.).

An exemplary expression system in mammalian cell lines may utilize adenovirus as an expression vector. Coding sequences may be ligated to, e.g., the adenovirus late promoter. The cloned gene may be inserted into the adenovirus genome by in vitro or in vivo recombination. Insertion in a non-essential region of the viral genome (e.g., region E1 or E3) results in a recombinant virus that is capable of infecting and expressing cloned proteins in: mammalian host cells. The disclosed examples are not limiting and any known expression vector may be used.

Expressed proteins may be partially or completely purified before analysis. In some embodiments of the invention, protein purification may be facilitated by expressing cloned sequences as fusion proteins containing short leader sequences that allow rapid affinity purification. Examples of such fusion protein expression systems are the glutathione S-transferase system (Pharmacia, Piscataway, N.J.), the maltose binding protein system (NEB, Beverley, Mass.), the FLAG system (IBI, New Haven, Conn.), and the 6×His system (Qiagen, Chatsworth, Calif.). In one embodiment of the invention, the leader sequence is linked to a protein by a specific recognition site for a protease. Examples of suitable sequences include those recognized by the Tobacco Etch Virus protease (Life Technologies, Gaithersburg, Md.) or Factor Xa (New England Biolabs, Beverley, Mass.). Alternatively, expressed proteins may be purified by standard techniques discussed above.

Although the methods disclosed above are directed towards analysis of proteins, they are also applicable to the analysis of other types of biomolecules. For example, cells could be incubated in a labeled monosaccharide and polysaccharides could be purified and analyzed as described herein.

Binding Moieties

In some embodiments of the invention, the target biomolecule(s) of interest may be captured, immobilized and/or labeled by binding to one or more binding moieties. A variety of moieties are known in the art, including but not limited to oligonucleotides, nucleic acids, aptamers, antibodies, antibody fragments, chimeric antibodies, single-chain antibodies, ligands, binding proteins, receptor proteins, inhibitors, substrates, etc. Any such known binding moiety may be used in the claimed methods. Exemplary binding moieties—antibodies and aptamers—are discussed in further detail below. Methods for design and production of oligonucleotide binding moieties, e.g. for hybridization to a target nucleic acid and/or oligonucleotide tag, are known in the art and are similar to the methods for primer production discussed above.

Antibodies

Methods for preparing and characterizing antibodies are well known in the art (see, e.g., Harlow and Lane, 1988). Antibodies of use may be monoclonal or polyclonal. In preferred embodiments, monoclonal antibodies are used. Antibodies against a wide variety of antigens are available from commercial sources. Alternatively, antibodies against a novel target may be prepared as disclosed herein.

Antibodies are prepared by immunizing an animal with an immunogen (antigen) and collecting antisera from the immunized animal. A wide range of animal species can be used for the production of antisera. Typical animals used for production of polyclonal antibodies include, rabbits, mice, rats, hamsters, pigs or horses. Because of the relatively large blood volume of rabbits, a rabbit is a preferred choice for production of polyclonal antibodies, while mice are preferred for monoclonal antibody production.

Antibodies, both polyclonal and monoclonal, may be prepared using conventional immunization techniques, generally known in the art. A composition containing antigenic epitopes can be used to immunize one or more experimental animals, such as a rabbit or mouse, which will then produce specific antibodies against the antigens of interest. Polyclonal antisera may be obtained, after allowing time for antibody generation, simply by bleeding the animal and preparing serum samples from the whole blood.

As is well known in the art, a given composition may vary in its immunogenicity. It is often necessary to boost the host immune system, as may be achieved by coupling a peptide or polypeptide immunogen to a carrier. Exemplary carriers are keyhole limpet hemocyanin (KLH) and bovine serum albumin (BSA). Other albumins such as ovalbumin, mouse serum albumin or rabbit serum albumin also can be used as carriers. Techniques for conjugating a polypeptide to a carrier protein are well known in the art and include use of cross-linking reagents such as glutaraldehyde, m-maleimidobenzoyl-N-hydroxysuccinimide ester, carbodiimide and bis-biazotized benzidine. The immunogenicity of a particular immunogen composition may also be enhanced by the use of non-specific stimulators of the immune response, known as adjuvants. Exemplary adjuvants include complete Freund's adjuvant (a non-specific stimulator of the immune response containing killed Mycobacterium tuberculosis), incomplete Freund's adjuvant and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). Booster injections also may be given. The process of boosting and titering is repeated until a suitable titer is achieved. When a desired level of immunogenicity is obtained, the immunized animal can be bled and the serum isolated and stored, and/or the animal can be used to generate monoclonal antibodies.

Monoclonal antibodies may be readily prepared through use of well-known techniques, such as those exemplified in U.S. Pat. No. 4,196,26. Typically, this involves immunizing a suitable animal with a selected immunogen composition. Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell. Any one of a number of myeloma cells may be used, as are known to those of skill in the art (Goding, In: Monoclonal Antibodies: Principles and Practice, 2d ed., Academic Press, Orlando, Fla., pp. 60-61, and 71-74, 1986; Campbell, In: Monoclonal Antibody Technology, Laboratory Techniques in Biochemistry and Molecular Biology, Burden and. Von Knippenberg, Eds., Vol. 13:75-83, Elsevier, Amsterdam, 1984). For example, where the immunized animal is a mouse, one may use P3-X63/Ag8, P3-X63-Ag8.653, NS1/1.Ag 4 l, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus (Kohler and Milstein, Nature, 256:495-497, 1975; Eur. J. Immunol., 6: 511-519, 1976), and those using polyethylene glycol (PEG), such as 37% (v/v) PEG, have been disclosed by Gefter et al., (Somatic Cell Genet., 3: 231-236, 1977). The use of electrically induced fusion methods is also appropriate (Goding, 1986).

Fusion procedures usually produce viable hybrids at low frequencies, around 1×10⁻⁶ to 1×10⁻⁸. However, fused hybrids may be differentiated from the parental unfused cells by culturing in a selective medium. The selective medium generally contains an agent that blocks the de novo synthesis of nucleotides in the tissue culture media. Exemplary and preferred agents are aminopterin, methotrexate, and azaserine. Where aminopterin or methotrexate is used, the media is supplemented with hypoxanthine and thymidine as a source of nucleotides (HAT medium). Where azaserine is used, the media is supplemented with hypoxanthine. A preferred selection medium is HAT. The only cells that can survive in the selective media are those hybrids formed from myeloma and B-cells.

Typically, selection of hybridomas is performed by culturing the cells by single-clone dilution in microtiter plates, followed by testing the individual clonal supernatants (after about two to three wk) for the desired reactivity. The selected hybridomas may then be serially diluted and cloned into individual antibody-producing cell lines, which clones can be propagated indefinitely to provide mAbs.

Aptamers

In certain embodiments of the invention, the binding moieties to be used may comprise aptamers. Methods of constructing and determining the binding characteristics of aptamers are well known in the art. For example, such techniques are disclosed in Lorsch and Szostak (In: Combinatorial Libraries: Synthesis, Screening and Application Potential, R. Cortese, ed., Walter de Gruyter Publishing Co., New York, pp. 69-86, 1996) and in U.S. Pat. Nos. 5,582,981, 5,595,877 and 5,637,459. Aptamers may be comprised of DNA or RNA. Alternatively, once a given aptamer sequence has been identified, modified oligomers of the same sequence may be prepared to provide enhanced stability to nucleases. Any of the hydroxyl groups ordinarily present in oligonucleotides may be replaced by phosphonate groups, phosphate groups, protected by a standard protecting group, or activated to prepare additional linkages to other nucleotides, or may be conjugated to solid supports. The 5′ terminal OH is conventionally free but may be phosphorylated. Hydroxyl group substituents at the 3′ terminus may also be phosphorylated. The hydroxyls may be derivatized by standard protecting groups. One or more phosphodiester linkages may be replaced by alternative linking groups. These alternative linking groups include exemplary embodiments wherein P(O)O is replaced by P(O)S, P(O)NR₂, P(O)R, P(O)OR′, CO, or CNR₂, wherein R is H or alkyl (1-20C) and R′ is alkyl (1-20C); in addition, this group may be attached to adjacent nucleotides through O or S. Not all linkages in an oligomer need to be identical.

In preferred embodiments, the starting pool of oligonucleotides (referred to as nucleic acid ligands) used to prepare aptamers will contain a randomized sequence portion flanked by primer sequences that permit the amplification of nucleic acid ligands found to bind to a selected target. Both the randomized portion and the primer hybridization regions of the initial nucleic acid ligand population may be constructed using conventional solid phase techniques. Such techniques are well known in the art (e.g., Froehler, et al., Tet Lett. 27:5575-5578, 1986a; Nucleic Acids Research, 14:5399-5467, 1986b; Nucleosides and Nucleotides, 6:287-291, 1987; Nucleic Acids Research, 16:4831-4839, 1988). For synthesis of the randomized regions, mixtures of nucleotides at the positions where randomization is desired are added during synthesis.

A preferred method of selecting for selecting aptamers of specific binding activity involves use of the SELEX process, disclosed for example in U.S. Pat. No. 5,475,096 and U.S. Pat. No. 5,270,163. SELEX involves selection from a mixture of candidate nucleic acid ligands and step-wise iterations of binding, partitioning and amplification, using the same general selection scheme, to achieve any desired criterion of binding affinity and selectivity. Starting from a mixture of nucleic acid ligands, the method includes: Contacting the mixture with the target under conditions favorable for binding. Partitioning unbound nucleic acid ligands from those nucleic acid ligands that have bound specifically to target analyte. Dissociating the nucleic acid ligand-analyte complexes. Amplifying the nucleic acid ligands dissociated from the nucleic acid ligand-analyte complexes to yield a mixture of nucleic acid ligands that preferentially bind to the analyte. Reiterating the steps of binding, partitioning, dissociating and amplifying through as many cycles as desired to yield highly specific aptamers that bind with high affinity to the target analyte.

Labels

In certain embodiments of the invention, one or more labels may be attached to a binding moiety, probe, primer, target biomolecule or other molecule. A number of different labels may be used, such as fluorophores, chromophores, radioisotopes, enzymatic tags, antibodies, bioluminescent, electroluminescent, phosphorescent, affinity labels, nanoparticles, metal nanoparticles, gold nanoparticles, silver nanoparticles, magnetic particles, spin labels or any other type of label known in the art.

Non-limiting examples of affinity labels include an antibody, an antibody fragment, a receptor protein, a hormone, biotin, DNP, and any polypeptide/protein molecule that binds to an affinity label.

Non-limiting examples of enzymatic tags include urease, alkaline phosphatase or peroxidase. Colorimetric indicator substrates can be employed with such enzymes to provide a detection means visible to the human eye or spectrophotometrically.

Non-limiting examples of photodetectable labels include Alexa 350, Alexa 430, AMCA, aminoacridine, BODIPY 630/650, BODIPY 650/665, BODIPY-FL, BODIPY—R6G, BODIPY-TMR, BODIPY-TRX, 5-carboxy-4′,5′-dichloro-2′,7′-dimethoxy fluorescein, 5-carboxy-2′,4′,5′,7′-tetrachlorofluorescein, 5-carboxyfluorescein, 5-carboxyrhodamine, 6-carboxyrhodamine, 6-carboxytetramethyl amino, Cascade Blue, Cy2, Cy3, Cy5,6-FAM, dansyl chloride, Fluorescein, HEX, 6-JOE, NBD (7-nitrobenz-2-oxa-1,3-diazole), Oregon Green 488, Oregon Green 500, Oregon Green 514, Pacific Blue, phthalic acid, terephthalic acid, isophthalic acid, cresyl fast violet, cresyl blue violet, brilliant cresyl blue, para-aminobenzoic acid, erythrosine, phthalocyanines, azomethines, cyanines, xanthines, succinylfluoresceins, rare earth metal cryptates, europium trisbipyridine diamine, a europium cryptate or chelate, diamine, dicyanins, La Jolla blue dye, allopycocyanin, allococyanin B, phycocyanin. C, phycocyanin R, thiamine, phycoerythrocyanin, phycoerythrin R, REG, Rhodamine Green, rhodamine isothiocyanate, Rhodamine Red, ROX, TAMRA, TET, TRIT (tetramethyl rhodamine isothiol), Tetramethylrhodamine, and Texas Red. These and other luminescent labels may be obtained from commercial sources such as Molecular Probes (Eugene, Oreg.).

In other embodiments of the invention, labels of use may comprise metal nanoparticles. Methods of preparing nanoparticles are known. (See e.g., U.S. Pat. Nos. 6,054,495; 6,127,120; 6,149,868; Lee and Meisel, J. Phys. Chem. 86:3391-3395, 1982.) Nanoparticles may also be obtained from commercial sources (e.g., Nanoprobes Inc., Yaphank, N.Y.; Polysciences, Inc., Warrington, Pa.). Modified nanoparticles are available commercially, such as Nanogold® nanoparticles from Nanoprobes, Inc. (Yaphank, N.Y.).

In some embodiments of the invention, proteins may be labeled using side-chain specific and/or selective reagents. Such reagents and methods are known in the art. Non-limiting exemplary reagents that may be used include acetic anhydride (lysine, cysteine, serine and tyrosine); trinitrobenzenesulfonate (lysine); carbodiimides (glutamate, aspartate); phenylglyoxal (arginine); 2,3-butanedione (arginine); pyridoxal phosphate (lysine); p-chloromercuribenzoate (cysteine); 5,5′-dithiobis(2-nitro-benzoic acid) (cysteine); diethylpyrocarbonate (lysine, histidine); N-bromosuccinimide (tryptophan) and tetranitromethane (cysteine, tyrosine). In alternative embodiments of the invention, various cross-linking reagents known in the art, such as homo-bifunctional, hetero-bifunctional and/or photoactivatable cross-linking reagents may be used. Non-limiting examples of such reagents include bisimidates; 1,5-difluoro-2,4-(dinitrobenzene); N-hydroxysuccinimide ester of suberic acid; disuccinimidyl tartarate; dimethyl-3,3′-dithio-bispropionimidate; N-succinimidyl-3-(2-pyridyldithio)propionate; 4-(bromoaminoethyl)-2-nitrophenylazide; and 4-azidoglyoxal. Such reagents may be modified to attach various types of labels, such as fluorescent labels. The skilled artisan will realize that such cross-linking reagents are not limited to use with proteins, but may also be used with other types of molecules.

Methods of Immobilization

In various embodiments of the invention, binding moieties, capture probes or analytes of interest may be attached to a surface by covalent or non-covalent interaction. One means for promoting such attachments involves the use of chemical or photo-activated cross-linking reagents. Such reagents are well known in the art.

Homobifunctional reagents that carry two identical functional groups are highly efficient in inducing cross-linking. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking molecules are disclosed in U.S. Pat. No. 5,603,872 and U.S. Pat. No. 5,401,511. Various ligands can be covalently bound to surfaces through the cross-linking of amine residues. Amine residues may be introduced onto a surface through the use of aminosilane, for example. Coating with aminosilane provides an active functional residue, a primary amine, on the surface for cross-linking purposes. In another exemplary embodiment, the surface may be coated with streptavidin or avidin with the subsequent attachment of a biotinylated molecule, such as an antibody or analyte. To form covalent conjugates of ligands and surfaces, various cross-linking reagents have been used, including glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC).

In another non-limiting example, heterobifunctional cross-linking reagents and methods of using the cross-linking reagents are disclosed in U.S. Pat. No. 5,889,155. The cross-linking reagents combine, for example, a nucleophilic hydrazide residue with an electrophilic maleimide residue, allowing coupling in one example, of aldehydes to free thiols. The cross-linking reagent used can be designed to cross-link various functional groups. In various embodiments, the target biomolecules to be analyzed may be attached to a solid surface (or immobilized). Immobilization of biomolecules may be achieved by a variety of methods involving either non-covalent or covalent attachment. In an exemplary embodiment, immobilization may be achieved by coating a surface with streptavidin or avidin and the subsequent attachment of a biotinylated molecule (Holmstrom et al., Anal. Biochem. 209:278-283, 1993). Immobilization may also occur by coating a silicon, glass or other surface with poly-L-Lys (lysine), followed by covalent attachment of either amino- or sulfhydryl-modified biomolecule using bifunctional crosslinking reagents (Running et al., BioTechniques 8:276-277, 1990; Newton et al., Nucleic Acids Res. 21:1155-62, 1993).

Immobilization may take place by direct covalent attachment of 5′-phosphorylated nucleic acids to chemically modified surfaces (Rasmussen et al., Anal. Biochem. 198:138-142, 1991). The covalent bond between the nucleic acid and the surface is formed by condensation with a water-soluble carbodiimide. This method facilitates a predominantly 5′-attachment of the nucleic acids via their 5′-phosphates. DNA is commonly bound to glass by first silanizing the glass surface, then activating with carbodiimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule. DNA may be bound directly to membrane surfaces using ultraviolet radiation. Other non-limiting examples of immobilization techniques for nucleic acids are disclosed in U.S. Pat. Nos. 5,610,287, 5,776,674 and 6,225,068.

The type of surface to be used for immobilization is not limiting. In various embodiments, the immobilization surface may be magnetic beads, non-magnetic beads, a planar surface, or any other conformation of solid surface comprising almost any material. Non-limiting examples of surfaces that may be used include glass, silica, silicate, PDMS, silver or other metal coated surfaces, nitrocellulose, nylon, activated quartz, activated glass, polyvinylidene difluoride (PVDF), polystyrene, polyacrylamide, other polymers such as poly(vinyl chloride), poly(methyl methacrylate) or poly(dimethyl siloxane), and photopolymers which contain photoreactive species such as nitrenes, carbenes and ketyl radicals capable of forming covalent links with various molecules (See U.S. Pat. Nos. 5,405,766 and 5,986,076).

Statistical Signal Processing

In certain embodiments of the invention, statistical signal processing may be used to deconvolute a complex signal into its individual components. For example, some embodiments may involve sequencing of target nucleic acids, using a mixture of nucleotides that are distinguishably labeled with different fluorophores. In other embodiments, the presence of different target molecules in a sample may be simultaneously assayed using distinguishably probes that bind specifically to different targets. Such methods may benefit from the use of signal processing techniques disclosed herein. The signal processing techniques are generally applicable where a number of otherwise identical reactions or processes occur simultaneously, with variable temporal offset. This may occur, for example, where multiple copies of a DNA template are being simultaneously replicated. Although in preferred embodiments, all copies of a given template will be subject to a coordinated initiation of replication, random variations in the polymerization process will rapidly result in a distribution of reaction rates, with some complementary strands synthesized earlier and others synthesized later. The resulting temporal offset in signal detection will soon result in a highly convoluted signal that may preferably be deconvoluted before further analysis.

For a fixed signature signal s(t) of duration T seconds, i.e,

s(t)≠0 for 0≦t≦T (21)

and

s(t)=0 for t<0,t>T (22)

the random superposition of N such signatures immersed in noise may be observed. The observed signal may be described by

$$y ( t ) = {\sum }_{n = 1}^{N} s ( t - {d}_{n} ) + v ( t ) , \qquad ( 23 )$$

where dₙ represents the random delay (time shift) for the n th (n=1,2, . . . , N) signature sequence and where v(t) represents the noise process. It is assumed that the observed signal starts at time t=0, so that all the delays are non-negative (i.e, dₙ≧0).

In practice, continuous signals are rarely measured. Rather what are measured are the sampled values of the signal, obtained from sampling at a certain rate. With a sampling rate of R samples per second, the signature signal may be represented by the following sequence of length L=RT+1

sᵢ=s(i/R), i=0,1, . . . ,RT. (24)

In this case, the sampled observation signal yᵢ=y(i/R) is simply

$${y}_{i} = {\sum }_{n = 1}^{N} {s}_{i - {k}_{n}} + {v}_{i} , \qquad ( 25 )$$

where vᵢ=v(i/R) represents the samples of the noise and where kₙ represents the delay via the formula

kₙ=└Rdₙ┘. (26)

Equation 25 assumes that the sequence sᵢ is zero for i<0. An important condition for the present analysis is that N is very large. In this case it is reasonable to consider a distribution for the delays dₙ, or kₙ. If Nⱼ denotes the number of signature sequences that begin at time j, i.e., the number of n such that kₙ=j, Equation 25 may be rewritten as

$${y}_{i} = {\sum }_{j = 0}^{D} {N}_{j} {s}_{i - j} + {v}_{i} , \qquad ( 27 )$$

where D represents the total duration of the delays. In other words, the delays j extend from j=0 to j=D. Note, moreover, that

$${\sum }_{j = 0}^{D} {N}_{j} = N \qquad ( 28 )$$

and that the total duration of the observed signal is

D+RT+1. (29)

It is also possible to write the “convolution” in Equation 27 as

$${y}_{i} = {\sum }_{j = 0}^{RT} {N}_{i - j} {s}_{j} + {v}_{j} . \qquad ( 29 )$$

It is now possible to resolve the following problem. Given the observations sequence yᵢ, satisfying Equation 27, or equivalently Equation 29, determine the unknown signature sequence sᵢ. A standard assumption for the noise process vᵢ is that it is zero-mean, Gaussian and white, i.e., uncorrelated in time—although other types of noise models can also be dealt with, e.g., zero-mean Gaussian noise with a certain power spectral density function.

If the Nⱼ in Equation 27 or Equation 29 are assumed to be known then we are simply confronted with an overdetermined system of linear equations in the unknowns sᵢ. To see this more explicitly, it is useful to rewrite Equation 27 in the following form

$$[ \begin{matrix}{y}_{0} \\ {y}_{1} \\ ⋮ \\ {y}_{D + RT - 1} \\ {y}_{D + RT}\end{matrix} ] = [ \begin{matrix}{N}_{0} & & & \\ {N}_{1} & {N}_{0} & & \\ ⋮ & {N}_{1} & ⋰ & \\ {N}_{D} & ⋮ & ⋰ & {N}_{0} \\ & {N}_{D} & ⋮ & {N}_{1} \\ & & ⋰ & ⋮ \\ & & & {N}_{D}\end{matrix} ] [ \begin{matrix}{s}_{0} \\ {s}_{1} \\ ⋮ \\ {s}_{RT - 1} \\ {s}_{RT}\end{matrix} ] + [ \begin{matrix}{v}_{0} \\ {v}_{1} \\ ⋮ \\ {v}_{D + RT - 1} \\ {v}_{D + RT}\end{matrix} ] . \qquad ( 30 )$$

With the Nⱼ known, the coefficient matrix in Equation 30 is known. Therefore the unknown vector of sᵢ's can be readily computed via standard methods such as least-squares. The problem is that the Nⱼ are not known. All that is observed is the sequence yᵢ. Therefore we are confronted with an equation where all the quantities on the right-hand-side (the Nⱼ, sᵢ and vᵢ) are unknown. A natural question is whether in principle the desired sᵢ may be identified from Equation 30.

If it is assumed that the noise vector of vᵢ's is negligible, then Equation 30 is a system of D+RT+1 equations (the number of observations) in D+RT+2 unknowns (D+1 unknowns for the Nⱼ and RT+1 unknowns for the sᵢ). Therefore, even in the noiseless case, it appears that there is an identifiability problem for there are more unknowns than equations. Of course, it is possible to use the equation

$${\sum }_{j = 0}^{D} {N}_{j} = N$$

to get the number of equations and unknowns to match. However, with some very reasonable statistical assumptions it is possible to circumvent the idenitifiability problem altogether.

Statistical Assumptions: Exploiting Large N

A distinguishing feature of sequencing problems is that the number of DNA molecules, and hence signature sequences, N is extremely large. Therefore if something is known about the statistics of the delay distribution then it is possible to “estimate” the values of the Nⱼ, and thereby the coefficient matrix in Equation 30. The statistics of the delay distribution is a macroscopic quantity, and so it is reasonable to assume that it is known. Moreover, being a macroscopic quantity, it is also reasonable to assume that it may be controlled using an appropriate system design. This statistical knowledge can be used to estimate the Nⱼ.

Uniform delay distribution: Assume that the delay distribution is uniform over D, the duration of the delays. In other words, the signature sequences are equally likely to begin anywhere in the interval [0,D]. This assumption is true in many applications and the sequencing system may be designed to exhibit a uniform delay distribution over D.

Using properties of the binomial distribution, each of the N will be random variables with mean and variance

μ_{N}=ENⱼ=N/D and σ_{N}²=E(Nⱼ−N/D)²=(1−1/D)N/D (31)

where E denotes expectation. It can also be shown that the random variables Nⱼ have cross-covariance:

C_{N}_{ᵢ}_{N}_{ⱼ}=E(N−N/D)(Nⱼ−N/D)=−N/D². (32)

Equation 31 shows that as N grows larger the mean N/D becomes a better and better estimate of the actual value Nⱼ. The ratio of the standard deviation of Nⱼ to its mean is given by

$$\frac{{σ}_{N}}{{μ}_{N}} = \sqrt{\frac{D - 1}{N}} , \qquad ( 33 )$$

which goes to zero as N goes to infinity, so that the estimate becomes more and more reliable with larger sample size. If we define the random variable \overline{N}ⱼ=Nⱼ−N/D, Equation 30 may be rewritten as:

$$[ \begin{matrix}{y}_{0} \\ {y}_{1} \\ ⋮ \\ {y}_{D + RT - 1} \\ {y}_{D + RT}\end{matrix} ] = \frac{N}{D} [ \begin{matrix}1 & & & \\ 1 & 1 & & \\ ⋮ & 1 & ⋰ & \\ 1 & ⋮ & ⋰ & 1 \\ & 1 & ⋮ & 1 \\ & & ⋰ & ⋮ \\ & & & 1\end{matrix} ] [ \begin{matrix}{s}_{0} \\ {s}_{1} \\ ⋮ \\ {s}_{RT - 1} \\ {s}_{RT}\end{matrix} ] + [ \begin{matrix}{\tilde{N}}_{0} & & & \\ {\tilde{N}}_{1} & {\tilde{N}}_{0} & & \\ ⋮ & {\tilde{N}}_{1} & ⋰ & \\ {\tilde{N}}_{D} & ⋮ & ⋰ & {\tilde{N}}_{0} \\ & {\tilde{N}}_{D} & ⋮ & {\tilde{N}}_{1} \\ & & ⋰ & ⋮ \\ & & & {\tilde{N}}_{D}\end{matrix} ] [ \begin{matrix}{s}_{0} \\ {s}_{1} \\ ⋮ \\ {s}_{RT - 1} \\ {s}_{RT}\end{matrix} ] + [ \begin{matrix}{v}_{0} \\ {v}_{1} \\ ⋮ \\ {v}_{D + RT - 1} \\ {v}_{D + RT}\end{matrix} ] . \qquad ( 34 )$$

In Equation 34, the matrix coefficient in the first term is known. Although the second matrix coefficient is unknown its “energy” is less by a factor of N. To make this more precise, defining the last two terms in Equation 34 as an “equivalent” noise

$$[ \begin{matrix}{w}_{0} \\ {w}_{1} \\ ⋮ \\ {w}_{D + RT - 1} \\ {w}_{D + RT}\end{matrix} ] = [ \begin{matrix}{\tilde{N}}_{0} & & & \\ {\tilde{N}}_{1} & {\tilde{N}}_{0} & & \\ ⋮ & {\tilde{N}}_{1} & ⋰ & \\ {\tilde{N}}_{D} & ⋮ & ⋰ & {\tilde{N}}_{0} \\ & {\tilde{N}}_{D} & ⋮ & {\tilde{N}}_{1} \\ & & ⋰ & ⋮ \\ & & & {\tilde{N}}_{D}\end{matrix} ] [ \begin{matrix}{s}_{0} \\ {s}_{1} \\ ⋮ \\ {s}_{RT - 1} \\ {s}_{RT}\end{matrix} ] + [ \begin{matrix}{v}_{0} \\ {v}_{1} \\ ⋮ \\ {v}_{D + RT - 1} \\ {v}_{D + RT}\end{matrix} ] , \qquad ( 35 )$$

Using Equation 31 and Equation 32 it is straightforward to compute the covariance matrix of the equivalent noise. If the off-diagonal terms are ignored compared to the diagonal ones (from Equations 31 and 32 σ_{N}² is larger than C_{N}_{ᵢ}_{N}_{ⱼ} by a factor of D), then the covariance matrix can be written as

$${R}_{w} = [ \begin{matrix}{σ}_{v}^{2} + \frac{{NP}_{s}}{DRT} & & & & & & & & \\ & {σ}_{v}^{2} + \frac{2 {NP}_{s}}{DRT} & & & & & & & \\ & & ⋰ & & & & & & \\ & & & {σ}_{v}^{2} + \frac{{NP}_{s}}{D} & & & & & \\ & & & & ⋰ & & & & \\ & & & & & {σ}_{v}^{2} + \frac{{NP}_{s}}{D} & & & \\ & & & & & & ⋰ & & \\ & & & & & & & {σ}_{v}^{2} + \frac{2 {NP}_{s}}{DRT} & \\ & & & & & & & & {σ}_{v}^{2} + \frac{{NP}_{s}}{DRT}\end{matrix} ]$$

for D>RT and

$${R}_{w} = [ \begin{matrix}{σ}_{v}^{2} + \frac{{NP}_{s}}{DRT} & & & & & & \\ & {σ}_{v}^{2} + \frac{2 {NP}_{s}}{DRT} & & & & & \\ & & ⋰ & & & & \\ & & & {σ}_{v}^{2} + \frac{N ( D + RT )}{2 DRT} & & & \\ & & & & ⋰ & & \\ & & & & & {σ}_{v}^{2} + \frac{2 {NP}_{s}}{DRT} & \\ & & & & & & {σ}_{v}^{2} + \frac{{NP}_{s}}{DRT}\end{matrix} ]$$

for D<RT, where the noise variance is defined as Evᵢvⱼ=σᵥ²δᵢⱼ and the signature signal energy is defined as

$${P}_{s} = {\sum }_{i = 0}^{R T} {s}_{i}^{2} . \qquad ( 36 )$$

An important quantity is the “equivalent” signal-to-noise-ratio (SNR), which can be computed to be

$$\begin{matrix}SNR = \frac{{SNR}_{perfect}}{1 + \frac{D}{N} {SNR}_{perfect}} , \\ where & ( 37 ) \\ {SNR}_{perfect} = \frac{{N}^{2} {P}_{s}}{D ( D + RT ) {σ}_{v}^{2}} , & ( 38 )\end{matrix}$$

is the SNR when we have exact knowledge of the Nⱼ. As N goes to infinity, SNR approaches SNR_{perfect}. In other words, in the limit of large N, the system behaves as if the values of the Nⱼ are known. Thus, the macroscopic statistical knowledge allows circumvention of the identifiability problem.

The Wiener solution: Now that all the relevant covariance matrices have been computed, it is straightforward to find the least-mean-squares estimate of the signature sequence. The solution is referred to as the Wiener solution and is given by

$$[ \begin{matrix}\hat{{s}_{0}} \\ \hat{{s}_{1}} \\ ⋮ \\ \hat{{s}_{RT - 1}} \\ \hat{{s}_{RT}}\end{matrix} ] = \frac{{NP}_{s}}{DRT} {{Θ}^{*} ( {R}_{w} + \frac{{N}^{2} {P}_{s}}{{D}^{2} RT} Θ {Θ}^{*} )}^{- 1} [ \begin{matrix}{y}_{0} \\ {y}_{1} \\ ⋮ \\ {y}_{D + RT - 1} \\ {y}_{D + RT}\end{matrix} ] , \qquad ( 39 )$$

where the (D+RT+1)×(RT+1) Toeplitz matrix Θ from Equation 34 is defined as

$$Θ = [ \begin{matrix}1 & & & \\ 1 & 1 & & \\ ⋮ & 1 & ⋰ & \\ 1 & ⋮ & ⋰ & 1 \\ & 1 & ⋮ & 1 \\ & & ⋰ & ⋮ \\ & & & 1\end{matrix} ] . \qquad ( 40 )$$

The Wiener solution shown in Equation 39 requires computing the inverse of a (D+RT+1)×(D+RT+1) matrix. Due to the Toeplitz structure this can be done efficiently and in a numerically stable way. Examplary resolutions of the inverse matrix computation using the Wiener solution are provided below in the Examples section.

Nanopores

In certain embodiments of the invention, nanopores may be used to characterize one or more target biomolecules, to detect signals generated by the movement of target molecules through the nanopores. A nanopore may be a protein channel in a lipid bilayer or an extremely small isolated ‘hole’ in a thin, solid-state membrane. For a nanopore to be useful as a single molecule detector, its diameter must not be much larger than the size of the molecule to be detected. When a single molecule enters a nanopore in an insulating membrane, it causes changes in the nanopore's electrical properties that are readily detected with known electronic devices and circuits. Alternatively, nanopores associated with sensor layers, such as photodetector sensor layers, may be incorporated into an apparatus or system.

Fabrication of Nanopores

Fabrication of nanopores, individually or in arrays, may utilize any technique known in the art for nanoscale manufacturing. In certain embodiments of the invention, nanopores may be constructed on a solid-state matrix using known nanolithography methods, including but not limited to chemical vapor deposition, electrochemical deposition, chemical deposition, electroplating, thermal diffusion and evaporation, physical vapor deposition, sol-gel deposition, focused electron beam, focused ion beam, molecular beam epitaxy, dip-pen nanolithography, reactive-ion beam etching, chemically assisted ion beam etching, microwave assisted plasma etching, electro-oxidation, scanning probe methods, chemical etching, laser ablation, or any other method known in the art (E.g., U.S. Pat. No. 6,146,227).

In certain embodiments of the invention, channels or grooves may be etched into a semiconductor surface by various techniques known in the art including, but not limited to, methodologies using an STM/AFM tip in an oxide etching solution. After channels are formed, two semiconductor surfaces may be opposed to create one or more nanopores that penetrate the semiconductor. In other embodiments of the invention, STM tip methodologies may be used to create nanopores and other nanostructures using techniques known in the art. In alternative embodiments of the invention, scanning probes, chemical etching techniques, and/or micromachining may be used to cut micrometer-dimensioned or nanometer-dimensioned channels, grooves or holes in a semiconductor substrate.

Alternatively, nanopores may be made using a high-throughput electron-beam lithography system (e.g., Heyderman et al., Microelectronic Engineering, 2003). Electron-beam lithography may be used to write features as small as 5 nm on silicon chips. Sensitive resists, such as polymethyl-methacrylate, coated on silicon surfaces may be patterned without use of a mask. The electron-beam array may combine a field emitter cluster with a microchannel amplifier to increase the stability of the electron beam, allowing operation at low currents. In some embodiments of the invention, the SoftMask™ control system may be used to control electron-beam lithography of nanoscale features on a semiconductor chip substrate.

In alternative embodiments of the invention, nanopores may be produced using focused atom lasers (e.g., Bloch et al., “Optics with an atom laser 210 beam,” Phys. Rev. Lett. 87:123-321,1). Focused atom lasers may be used for lithography, much like standard lasers or focused electron beams. Such techniques are capable of producing micron scale or even nanoscale structures on a chip. In other alternative embodiments of the invention, dip-pen nanolithography may be used to form (e.g., Ivanisevic et al., “Dip-Pen Nanolithography on Semiconductor Surfaces,” J. Am. Chem. Soc., 123: 7887-7889,1). Dip-pen nanolithograpy uses AFM techniques to deposit molecules on surfaces, such as silicon chips. Features as small as 15 nm in size may be formed, with spatial resolution of 10 nm. Nanoscale pores may be formed by using dip-pen nanolithography in combination with regular photolithography techniques.

In other embodiments of the invention, ion-beam lithography may be used to create on a chip (e.g., Siegel, “Ion Beam Lithography,” VLSI Electronics, Microstructure Science, Vol. 16, Einspruch and Watts Eds., Academic Press, New York, 1987). A finely focused ion beam may be used to write nanoscale features directly on a layer of resist without use of a mask. Alternatively, broad ion beams may be used in combination with masks to form features as small as 100 nm in scale. Chemical etching, for example, with hydrofluoric acid, is used to remove exposed silicon or other chip material that is not protected by resist. The disclosed methods are not limiting, and nanopores may be formed by any method known in the art.

### EXAMPLES

### Example 1

### BRC Assay

Sample Preparation

Total RNA extracts may be obtained from blood, tissues or cell lines using commercially available kits (e.g., Ambion, Austin, Tex.; Qiagen, Valencia, Calif.; Promega, Madison, Wis.). cDNA may be synthesized using a SuperScript™ or other commercial kit (Invitrogen Life Technologies, Austin, Tex.). Where preferred, polyadenylated mRNA may be purified by oligo(dT) column chromatography or other known methods.

In an exemplary embodiment, first strand cDNA synthesis employed an RNA/primer mixture containing 5 μl total RNA and 1 μl of 0.5 μg/μl oligo(dT) random primer or gene specific primer, incubated at 70° C. for 10 min and then placed on ice for at least 1 min. A reaction mixture containing 2 μl 10× buffer (0.1 M Tris-Acetate pH 7.75, 5 mM EDTA, 50 mM Mg-acetate, 2 mM kinase free dNTP and 0.1 M dithiothreitol) in which dATP was replaced with x-thio dATP was added to the RNA/primer mixture, mixed gently, collected by brief centrifugation and then incubated at 42° C. for 5 min. After addition of 200 U of SuperScript II reverse transcriptase, the tube was incubated at 40° C. for 15 min. The reaction was terminated by heating at 70° C. for 15 min and then chilling on ice. The dNTP used in cDNA synthesis should be kinase free. In preferred embodiments dATP is replaced with alpha-thio dATP or analogs that are not good substrates for luciferase.

An aliquot of synthesized cDNA was added to 50 μl of reaction mixture (see Ronaghi et al., Anal. Biochem. 242:84-89, 1996 with modifications) containing 250 ng luciferase (Promega, Madison, Wis.), 50 mU ATP sulfurylase (Sigma Chemical Co., St. Louis, Mo.), 2 mM dithiothreitol, 100 mM Tris-Acetate pH 7.75, 0.5 mM EDTA, 0.5 mg BSA, 0.2 mg polyvinylpyrrolidone (Mr 360.000), 10 μg D-luciferin (Biothema, Dalaro, Sweden), 5 mM magnesium acetate and 10 attomole to 0.01 attomole purified pyrophosphate or ATP. The addition of very low amounts of pyrophosphate or ATP (or analogs) was found to decrease background light emission from the reaction mixture. Although the precise mechanism is unknown, BRC performed without adding small amounts of ATP or PPi consistently exhibited background luminescence that precluded accurate measurement of target nucleic acids present in amounts of about a femtomole or lower. Inorganic pyrophosphate present in the cDNA sample as a result of polymerase mediated dNTP incorporation was converted to ATP by sulfurylase. The ATP was used to generate light in a luciferin/luciferase reaction.

The generated light intensity over a time interval may be used to calculate the number of target molecules converted to cDNA by reverse transcriptase. In this exemplary process, the total amount of polyadenylated RNA present in the sample was determined, using oligo(dT) random primers. The presence of specific target nucleic acids may be determined using sequence specific primers, as detailed below.

Synthesis and Purification of Sequence Specific Oligonucleotide Primers

The following oligonucleotides were synthesized and HPLC purified by MWG Biotech (High Points, N.C.).

| | | |
|---|---|---|
| B-MBPup | Biotin-5′-CGGCGATAAAGGCTATAACGG-3′ | (SEQ ID NO:1) |
| MBPup | 5′-CGGCGATAAAGGCTATAACGG-3′ | (SEQ ID NO:2) |
| B-MBPR1 | Biotin-5′-CTGGAACGCTTTGTCCGGGG-3′ | (SEQ ID NO:3) |
| MBPR1 | 5′-CTGGAACGCTTGTCCGGGG-3′ | (SEQ ID NO:4) |
| oligo-loop | 5′TTTTTTTTTTTTTTTTTTTTGCTGGAATTCGTCAGACTGGCCGTCGTTT | (SEQ ID NO:5) |
| TACAACGGAACGGCAGCAAAATGTTGC-3′ | | |

Template Preparation

Biotinylated PCR products were prepared from bacterial extracts containing pMAL vector (New England Biolabs, Beverly, Mass.) (Pourmand et al. 1998, Autoimmunity 28; 225-233) by standard techniques, using MBPup and biotinylated B-MBPR1 or MBPR1 and biotinylated B-MBPup as PCR primers. The PCR products were immobilized onto streptavidin-coated superparamagnetic beads (Dynabeads™ M280-Streptavidin, Dynal A. S., Oslo, Norway). Single-stranded DNA was obtained by incubating the immobilized PCR product in 0.10 M NaOH for 3 min to separate strands and then removing the supernatant.

Strand Extension

The immobilized single stranded PCR product was resuspended in annealing buffer (10 mM Tris-acetate pH 7.75, 2 mM Mg-acetate) and placed into wells of a microtiter plate. Five pmol of the BRC primers MBP-up (SEQ ID NO:2) or MBPR1 (SEQ ID NO:4) were added to the immobilized strand obtained from the PCR reaction (depending on what set of biotinylated PCR primers was used). Hybridization of the template and primers was performed by incubation at 95° C. for 3 min, 55° C. for 5 min and then cooling to room temperature. Extension occurred in the presence of 10 U exonuclease-deficient (exo-) Klenow DNA polymerase (New England Biolabs, Beverly, Mass.) and addition of all four deoxynucleoside triphosphates to the extension mixture (0.14 mM final concentration). As discussed above, α-thio dATP was substituted for dATP to prevent interference with the luciferase reaction. After extension, the contents of each well were serially diluted for comparison of light emission as a function of PPi concentration.

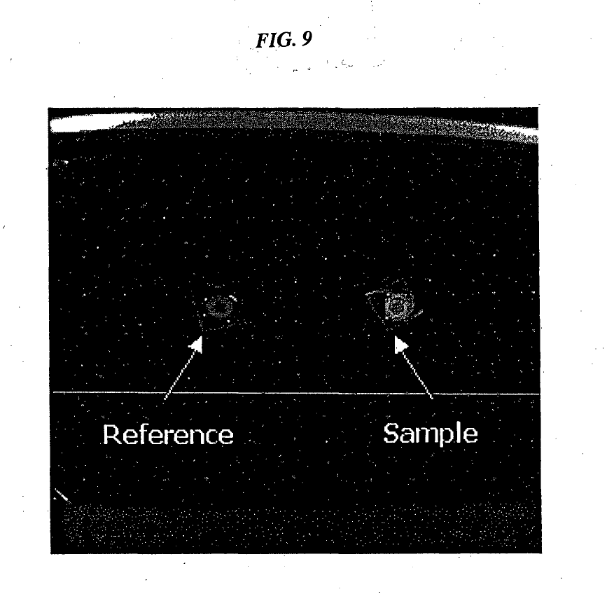

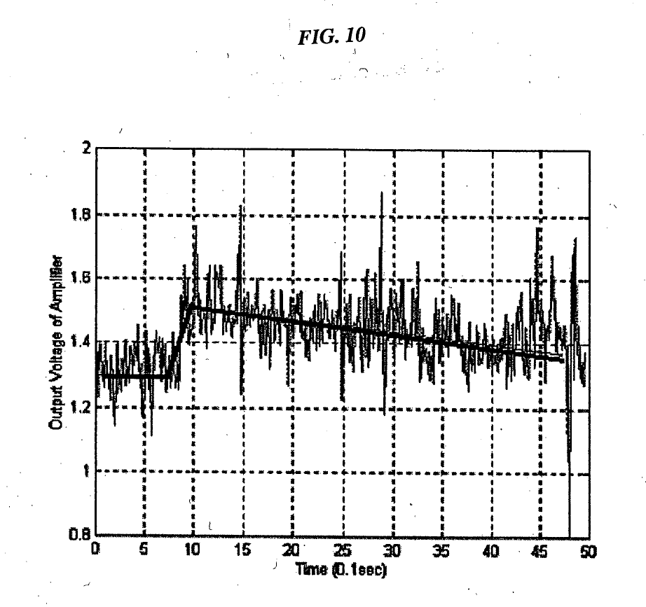

In an exemplary embodiment, extension and real-time luminometric monitoring were performed at 25° C. in an IVIS™ imaging system (Xenogen, Alameda, Calif.) or in and Lmax™ microplate luminometer (Molecular Devices, Sunnyvale, Calif.). A luminometric reaction mixture was added to the substrate with different concentrations of extended primed single-stranded DNA or self primed oligonucleotide. The luminometric assay mixture (40 μl) contained 3 μg luciferase (Promega, Madison, Wis.), 50 mU recombinant ATP sulfurylase (Sigma Chemicals, St. Louis, Mo.), 0.1 M Tris-acetate (pH 7.75), 0.5 mM EDTA, 5 mM Mg-acetate (Sigma Chemicals), 0.1% (w/v) bovine serum albumin (Sigma), 2.5 mM dithiothreitol (Sigma), 10 μM adenosine 5′-phosphosulfate (APS) (Biolog, Alexis Biochemicals, Carlsbad, Calif.), 0.4 mg polyvinylpyrrolidone/ml (molecular weight 360000) and 100 μg D-luciferin/ml (BioThema AB, Haninge, Sweden). Emitted light was detected in real-time and measured after approximately 45 seconds with 1 second and 10 second integration times for the CCD imaging system and luminometer, respectively. FIG. 9 shows a Xenogen image and amplified signal output for a 0.1 picomole sample of target nucleic acid. Similar images have been obtained with target nucleic acid samples as low as 0.1 attomole. Note that using the modified protocol with 0.01 attomole to 10 attomole purified pyrophosphate or ATP added, the background light intensity is essentially zero. FIG. 10 shows an increase in steady state light emission from a 10 fmol sample analyzed by BRC. FIG. 10 shows that even in the presence of random noise background that is of approximately the same order of magnitude as the actual signal, the pyrophosphate induced signal can still be detected as a shift in the baseline level of the light output.

The light coupling efficiencies of each system (including path loss) from the microtiter plate where the DNA samples were located to the sensor were approximately 0.012% and 8% for the CCD and PMT systems, respectively. In the CCD imaging system, a 96-well microtiter plate with multiple DNA samples was placed 18 cm below the lens of the camera, and in the luminometer a 384-well microtiter plate was inserted in the instrument chamber, where a PMT directly moves into close proximity (1 cm) of the sample for reading.

Detection Devices

The photosensitive device is typically either in direct proximity of the BRC reaction to directly receive incident photons, or relatively far from the buffer with a light coupling device (e.g. optical fiber or mirror system) capable of directing light from the sample to the detector. In an exemplary embodiment, a UDT-PIN-UV-50-9850-1 photodiode (Hamamatsu Corp., Hamamatsu, Japan) was used with a transimpedance amplifier with a gain of 10⁸ volts/amp.

### Example 2

### Detection of Target Nucleic Acids by BRC

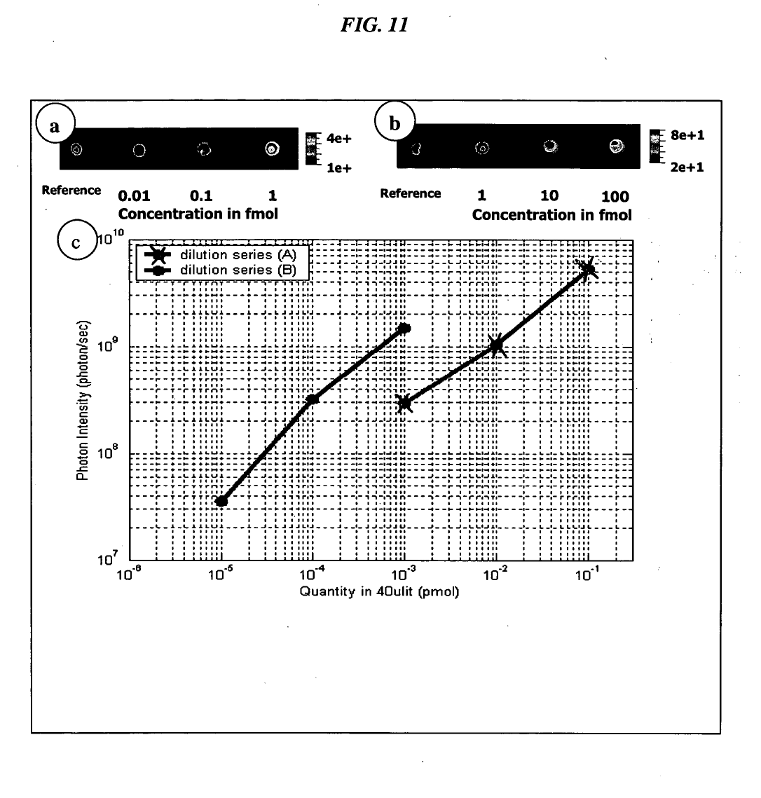

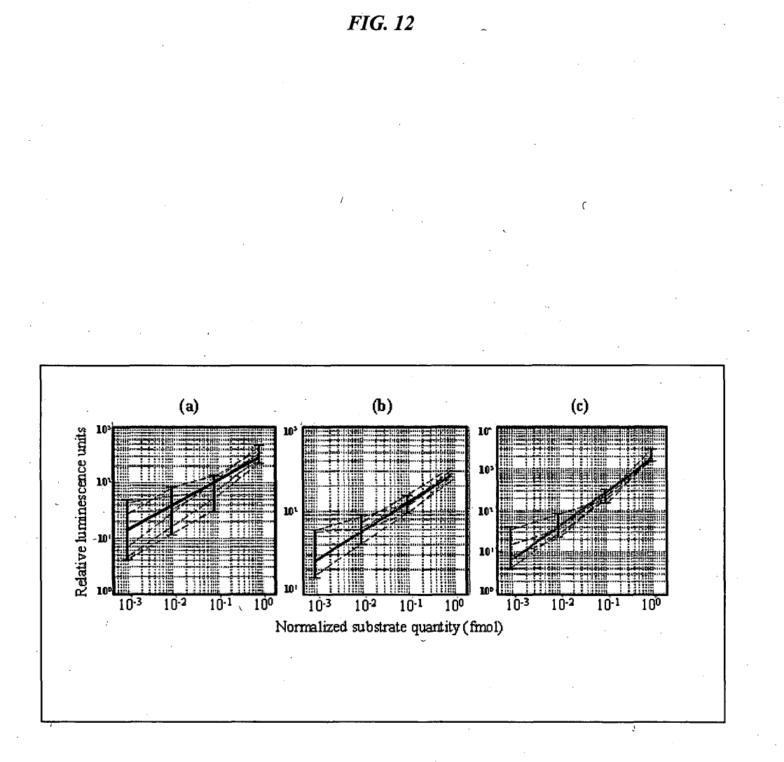

Detection of target nucleic acids by BRC assay was performed as described in Example 1, using a cooled CCD camera for light measurements. The signal obtained from 10 attomole to 100 femtomole of selected target molecules was determined. The target molecules, comprising either a synthesized oligonucleotide-loop or a 230 bp PCR product, were detected in 40 μl reaction volumes (FIG. 11). The same type of study was done using a standard luminometer. The performance of the two systems with a modified integration time (1 sec in CCD and 10 sec in luminometer) was compared (FIG. 11 vs. FIG. 12). These studies demonstrated the ability to detect 1 amol to 100 amol of target in 20 μl for both the oligonucleotide-loop and the 230 bp PCR product (MBP) with the luminometer.

The sensitivity of 1 amol observed in the BRC assay corresponds to approximately one million free pyrophosphate molecules in the solution, which is an extremely low concentration for 20 μl. If a given target DNA sequence has an extendable length of 1000 base pairs (which is a conservatively low number), then the disclosed sensitivity should allow detection of 1000 target DNA molecules using a single specific primer.

### Example 3

### Detection of Pathogen Nucleic Acids by Real-Time PCR Using BRC

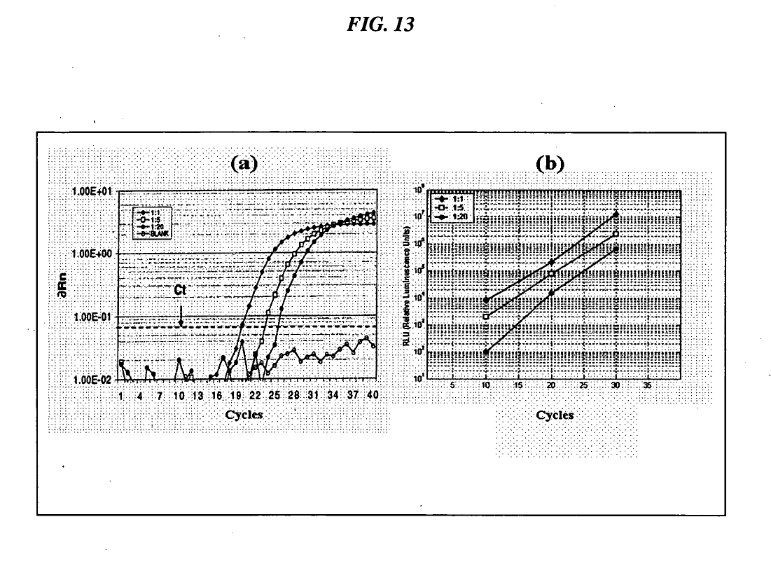

The BRC assay was performed using real-time quantitative PCR (RT-PCR) methods, in comparison with standard RT-PCR (Taqman™ assay, Applied Biosystems, Foster City, Calif.). A dilution series of cDNA from S. invicta Queen GP-9B was quantified using RT-PCR with the Taqman™ assay and BRC. As shown in FIG. 13, the sensitivity of BRC was better than Taqman™, using one tenth of the starting material and 10 less PCR cycles. The end point measurement sensitivity of BRC, based on the above result is at least 1000 better than the fluorescence based Taqman™ RT-PCR method.

### Example 4

### Measurement of Endogenous ATP Content by BRC

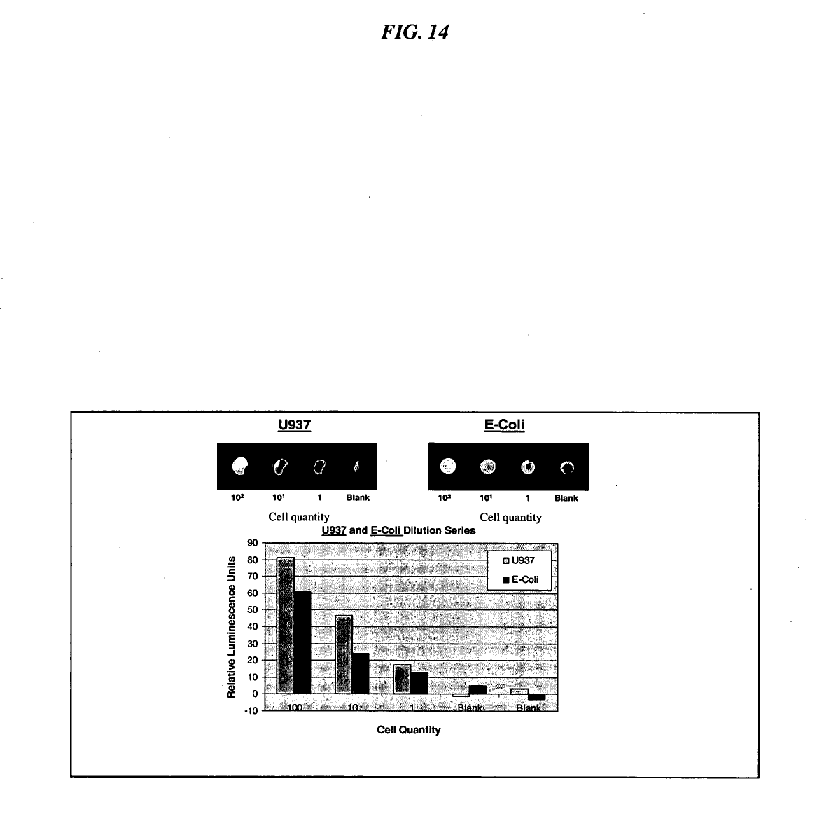

In certain embodiments of the invention, the amount of cells or microorganisms in a sample may be quantified by assaying for endogenous ATP and/or PPi. In an exemplary embodiment, the relative number of cells present was determined by employing BRC detection with samples comprising a dilution series of cell lysates from U937 macrophages (FIG. 14a) or E. coli (FIG. 14b). Even when diluted to a point where there was (on average) lysate from only one cell present, the BRC assay showed a detectable signal above background (FIG. 14). This indicates that the BRC detection assay can determine the presence of as few as 1-10 cells (equivalent to a few million total ATP molecules). More generally, BRC basedtATP and/or PPi detection may be used to quantify anywhere from 1 to 10,000 cells or microorganisms.

### Example 5

### SNP detection Using Total RNA Templates

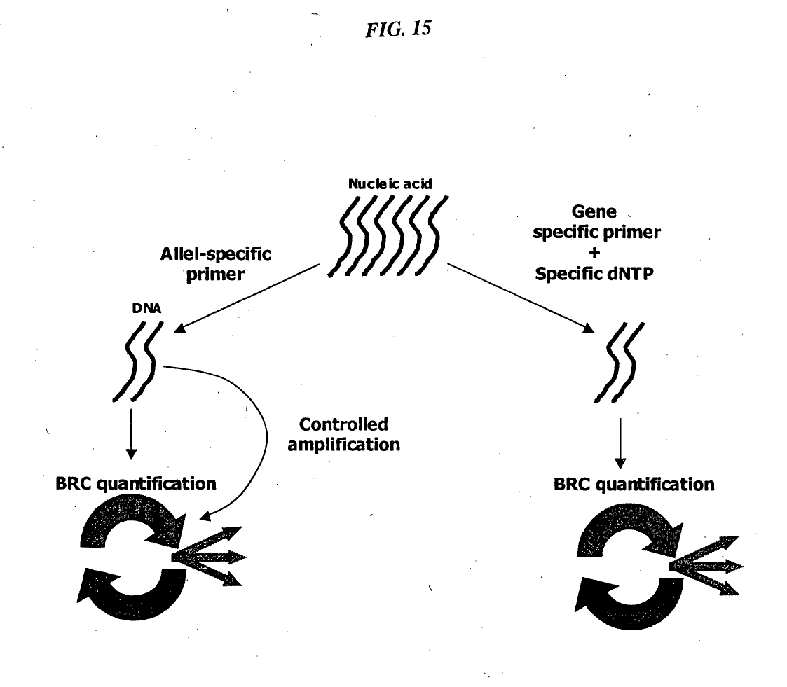

SNPs have been detected by hybridization of total RNA incubated with gene specific or allele specific primers and/or probes (Higgins et al, Biotechniques 23:710-714, 1997; Newton et al. Lancet 2:1481-1483, 1989; Goergen et al, J Med Virol 43:97-102, 1994; Newton et al, Nucleic Acids Res 17:2503-2516, 1989). Using the methods disclosed herein, SNPs may be detected by BRC, using sequence specific extension primers designed to bind to the template with the 3′ end of the primer located over the base of interest (SNP site) (FIG. 15). In preferred embodiments, the primer sequence is selected so that the end of the primer to which nucleotides will be attached is base-paired with the polymorphic site.

In certain embodiments, where the SNP is located in a coding sequence, the primer may be allowed to hybridize to total RNA or polyadenylated mRNA. (Alternatively, to detect non-coding SNPs genomic DNA or PCR amplified genomic DNA may be used as the target.) The template/primer fragments are used as the substrate for a primer extension reaction (e.g., Sokolov, Nucleic Acids Res 18:3671, 1989) in the presence of reverse transcriptase. If a target sequence is present that is complementary to the sequence specific primer, extension occurs and pyrophosphate is generated. An aliquot of the reaction product is added to a BRC reaction mixture as disclosed above. Extension products (PPi) are detected as disclosed above, allowing identification of the SNP in the target nucleic acid.

Typically SNPs exist in one of two alternative alleles. The allelic variant of the SNP may be identified by performing separate BRC reactions with primers specific for each of the SNP variants. In an alternative embodiment, the SNP allele may be identified using a gene specific primer that binds immediately upstream of the SNP site, allowing extension to occur in the presence of a single type of dXTP (or α-thio dATP) (FIG. 15). Extension will occur if the added DXTP is complementary to the SNP nucleotide.

### Example 6

### SNP Detection Using cDNA Templates

In alternative embodiments, SNPs may be detected from cDNA templates. Complementary DNAs may be prepared by standard methods, as disclosed above, and hybridized with gene specific or allele specific primers (FIG. 15) in 20 mM Tris-HCl (pH 7.5), 8 mM MgCl₂ or other standard conditions. The primers are designed to bind to the template with the 3′ end located over the polymorphic position. The template/primer fragments are then used as substrates in a primer extension reaction, as discussed above. Pyrophosphate generation, detected by the BRC reaction, indicates the presence of a SNP sequence that is complementary to the primer. As discussed above, gene specific primers also may be used in combination with single dXTPs.

### Example 7

### Pathogen Typing by BRC

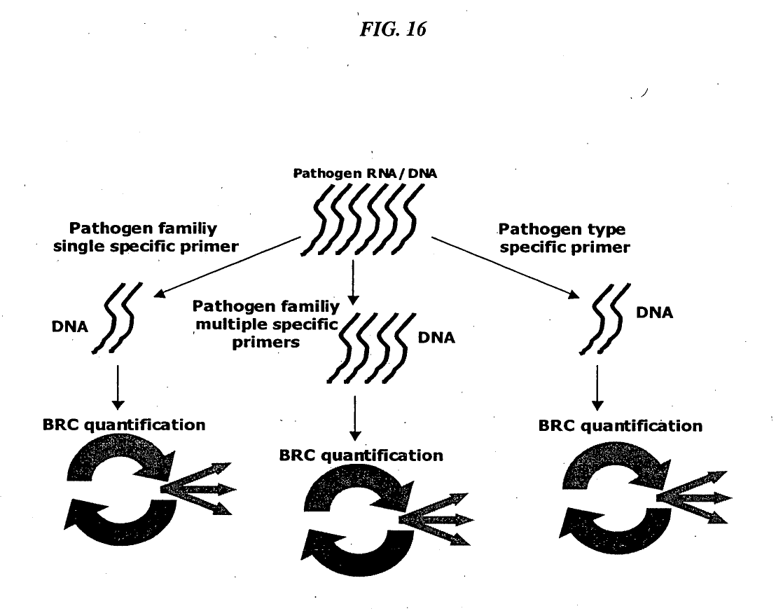

FIG. 16 illustrates embodiments of the invention in which BRC can be used to identify, type and/or quantify target pathogens in a sample. Total RNA or genomic DNA of the pathogenic organism may be incubated with pathogen specific primers (FIG. 16). In some embodiments, a single primer may be specific for one type of pathogen, or may be specific for a family of pathogens. Alternatively, multiple primers specific for different sub-types of a family of pathogens may be used. After hybridization in a suitable buffer, primer extension occurs with either reverse transcriptase or DNA polymerase, as disclosed above. The presence of a target pathogen type, or a member of a family of pathogens, is detected by luminescence using BRC. The pathogen titer (number of pathogenic organisms) in the sample may be determined by photon integration over a time interval, as discussed above.

### Example 8

### Pathogen Typing by Rolling Circle

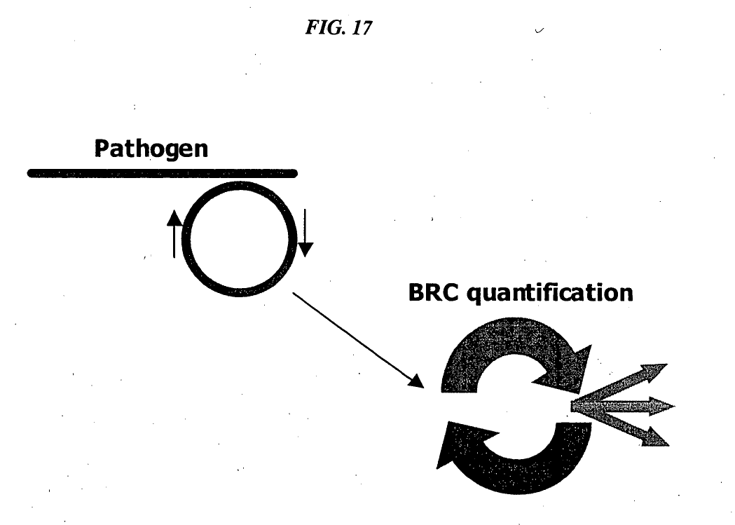

In various embodiments, BRC may be performed using a rolling circle replication process (FIG. 17). In this case, a circular primer sequence is allowed to hybridize with either total RNA or genomic DNA, for example of a pathogen. (Banér et al, Nucleic Acids Research, 26:5073-5078, 1998). As discussed above, the primer may be specific for a single type of pathogen, or may react with a family of pathogenic organisms. Alternatively, multiple circular primers specific for different members of a family of pathogens may be used. After hybridization, an exonuclease is added to the solution. The exonuclease digests single-stranded RNA or DNA, leaving intact double stranded RNA or DNA. The double stranded nucleic acid acts as the substrate in a primer extension reaction as discussed above, using reverse transcriptase or DNA polymerase. Formation of PPi is monitored by BRC.

### Example 9

### Protein-Protein Interaction

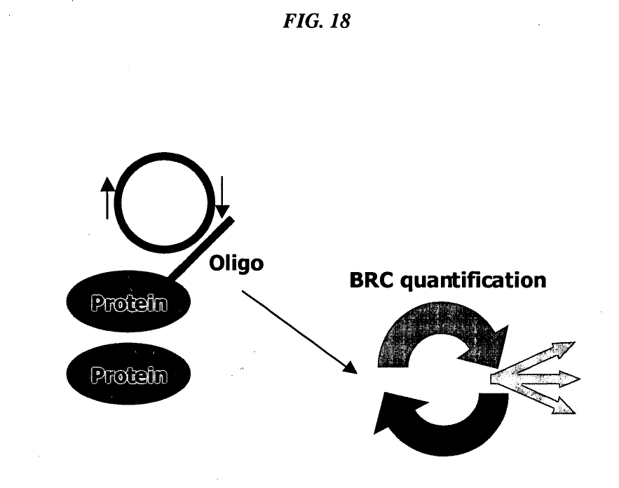

In some embodiments, BRC may be used to detect and/or quantify protein-protein binding (FIG. 18). A set of putative target proteins may be immobilized onto a surface, such as a nitrocellulose or nylon membrane or microtiter plate. A protein or peptide that binds to the target protein may be tagged with a short oligonucleotide, for example using a bifunctional cross-linking reagent. The oligonucleotide-tagged protein or peptide may be incubated with the putative target proteins under conditions allowing binding to occur. The remaining unbound proteins may be washed away and the presence of bound oligonucleotide detected by rolling circle reaction as discussed above (Baner et al., 1998), using circular oligonucleotide primers which are complementary to the short oligonucleotide tag. BRC may be used to detect and/or quantify the number of bound target proteins. The skilled artisan will realize that the disclosed method is not limited to protein-protein interactions, but may be applied to any binding pair interaction where one member of the pair may be tagged with a short oligonucleotide. The method may also be applied to arrays of putative target proteins, for example where in vitro translation has been used to create an array of candidate binding proteins from mRNAs.

### Example 10

### Gene Expression Profiling by Using Total RNA or cDNA

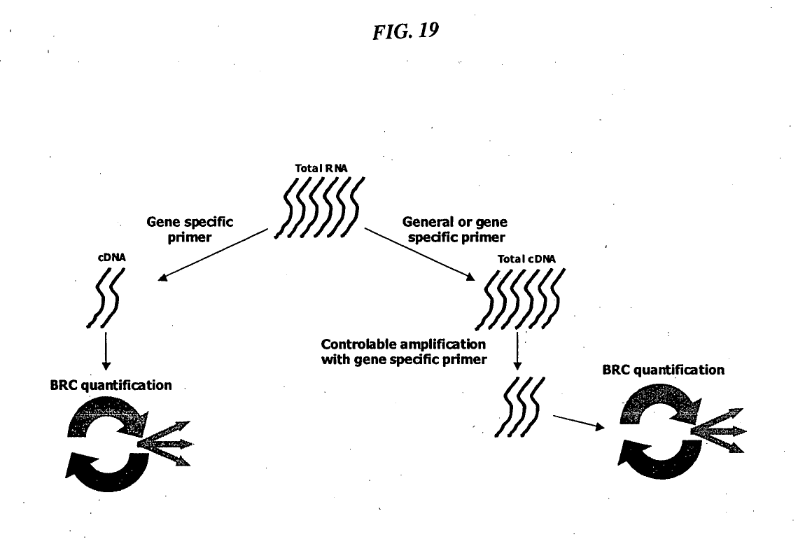

Total RNA or cDNA may be incubated with one or more gene specific primers or general primers (FIG. 19). Bound primer/template pairs are extended by reverse transcription or DNA polymerization. Formation of pyrophosphate is detected by BRC, as discussed above, and the amount of target nucleic acid may be quantified. In certain embodiments, a primer is, used that is designed to bind specifically to a single gene product (mRNA species), allowing determination of the level of expression for an individual gene. In other embodiments, non-specific primers, such as oligo(dT) and/or random primers may be used. In this case, the mRNA species present in a sample may be first separated, for example by hybridization to a DNA microarray containing complementary sequences for a large number of gene products. Hybridization may be followed by non-specific primer binding, extension and BRC reaction. Alternatively, the oligonucleotides of the: array may themselves be used as primers, allowing extension and light emission to occur. In such embodiments, the PPi reaction product may preferably be localized so that light emission is limited to the immediate location of a hybridized target nucleic acid. Many such localization techniques are known in the art, for example using microtiter plates wherein each well contains a probe for an individual gene expression product, or using a commercial apparatus such as a Nanochip® Workstation (Nanogen, San Diego, Calif.).

### Example 11

### Real time PCR

There are a variety of applications in which quantification of the amount of PCR reaction products in real time may be desired. The quantification of amplified target in a polymerase chain reaction (PCR) is achieved by incorporation of dNTP. As a result of dNTP incorporation PPi is released. An aliquot of synthesized DNA from each PCR cycle is added to a reaction mixture containing luciferase as disclosed above and thereby one can evaluate/estimate the mass of the molecules for each cycle from the generated light.

### Example 12

### Isothermal or Thermal Amplification of Nucleic Acids and BRC

A variety of nucleic acid amplification methods can be used in combination with biomolecule detection. Genomic DNA, cDNA, mRNA or total cell RNA may be extracted, mixed with appropriate reagents for amplification and, for example, BRC reagents for detection and quantification in the same tube. In certain embodiments, the amplification step may be performed separately from detection and quantification.

Polymerase Chain Reaction (PCR) Amplification

Genomic DNA is extracted, combined with DNTP, Mg, buffer, Taq Polymerase enzyme and sequence specific primers. The samples are cycled through 1-30 rounds of denaturation at 95° C., annealing at 40-70° C. and extension at 72° C. An aliquot of the PCR amplified sample is added to BRC assay mix and the amount of PPi generated quantified as a measure of the number of starting copies of sequence specific DNA present in the sample. Alternatively the PCR step can be combined with the BRC assay in one tube using a thermostable luciferase enzyme and ATP sulfurylase enzyme, as discussed above. In this method there is a coupling of amplification and detection/quantification of the target sequence. The number of PPi released in solution as a result of amplification is directly proportional to the length of the target sequence, and can be used to quantify the number of starting target nucleic acid in solution.

Results

Genomic DNA was amplified with primers specific to Maltose Binding Protein (MBP). An aliquot of the PCR product was diluted serially and assayed using the BRC method. Images were taken with standard CCD sensor with 1 sec integration time (not shown). Alternatively a luminometer was used with 10 sec integration time (not shown).

BRC was used with a complex genomic background with and without amplification steps. A bacterial colony containing the Rho 52 gene in a plasmid was grown on an agar plate. A colony with less than 100,000 bacteria was isolated and placed into 4 tubes containing buffer. The tubes were heated to 95° C. for 5 minutes and then the master mix containing Taq Polymerase, dNTPs, primers specific to Rho 52 and Mg was added. Tube 1 was heated to 95° C. for 1 min, 55° C. for 1 min and 72° C. for 1 minute for one amplification cycle. Tubes 2, 3 and 4 were amplified using similar temperatures but for 10 cycles, 20 cycles, and 30 cycles, respectively. An aliquot of each was added to the BRC assay for PPi measurement. Using the disclosed methods, target DNA was detected and quantified in each tube (not shown). Reference samples had all reagents and biological substances except primers.

Other potential isothermal applications that may be combined with BRC include Ribo-SPIA (Nugen Technologies), NASBA, RCA (Amersham), Ebervine (Ambion), Invader (Third Wave Techonologies) as well as cleavage based assays.

### Example 13

### Cloned Sequence Insert Detection Using BRC

The BRC procedure may be used to detect a given sequence of nucleic acid that has been inserted into a plasmid or other vector. In exemplary embodiments, a sequence of interest may be amplified from genomic DNA and purified. The amplified product is cloned into a plasmid and transfected into a bacteria. The bacterial is spread on an agar plate and allowed to incubate for a period of time and colonies are formed. A sample of a colony is picked and added to a tube containing buffer. The tube is heated for 5 minutes at 95° C. and then cooled.

BRC Analysis With Isothermal Amplification

BRC assay reagents and isothermal/thermal amplification reagents are added together into the tube with target sequence specific primer(s) and amplified at the appropriate temperature. Light intensity is measured for presence or absence of target sequence.

BRC Analysis With PCR

PCR reaction mixture was added to the tube contents along with a primer specific to the target sequence. The sample was subjected to one or more cycles of PCR amplification, for example at 95° C. (1 min), 55° C. (1 min), and 72° C. (1 min). In an illustrative embodiment, PCR amplification was performed for 0, 10, 20 or 30 cycles using a RO 52 sequence inserted into a plasmid vector. An aliquot was added to BRC reagents and light intensity was measured for presence or absence of target sequence.

Results

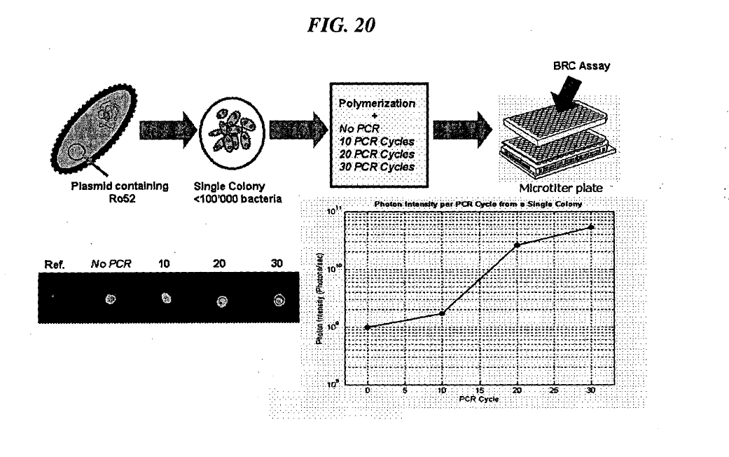

FIG. 20 shows that BRC can be used with a complex genomic background with and without amplification steps. Bacterial colonies containing a RO 52 sequence inserted into a standard plasmid vector were grown on an agar plate. A colony with less than 100,000 bacteria was isolated and placed into 4 tubes containing buffer. The tubes were heated to 95° C. for 5 minutes and then the master mix containing Taq Polymerase, dNTP, primers specific to RO 52 and Mg (2.5 mM MgCl₂) was added. Tube 1 was heated to 95° C. for 1 min, 55° C. for 1 min and 72° C. for 1 minute for one cycle. Tubes 2, 3 and 4 were subjected to similar temperature cycles but respectively for 10 cycles, 20 cycles, and 30 cycles. An aliquot of each was added to the BRC assay for PPi measurement. The target RO 52 insert sequence could be detected and quantified in each tube after zero, 10, 20 and 30 cycles of amplification. A reference sample containing all reagents and biological substances except the RO 52 specific primers showed no detectable signal (FIG. 20).

### Example 14

### Statistical Data Analysis for Nucleic Acid Sequencing

The signals considered in the following Example are in no way meant to model the actual signals obtained from sequencing DNA molecules. Instead, they provide an exemplary construct for demonstrating the use of the subject statistical analysis techniques to deconvolute complex signals. It is expected that the signals detected during sequencing will differ, depending on the measurement technique (nanopore, charge perturbation, bioluminescence regeneration, etc.) being employed. However, it is also expected that whatever detection system is used, the signals generated by incorporation of different types of nucleotide (A, T, G, C) will be distinguishable from each other.

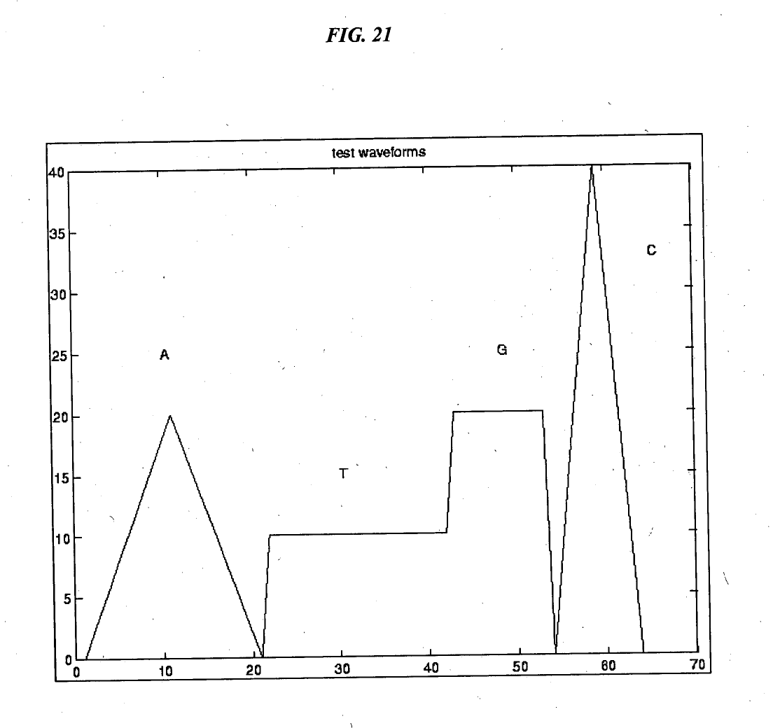

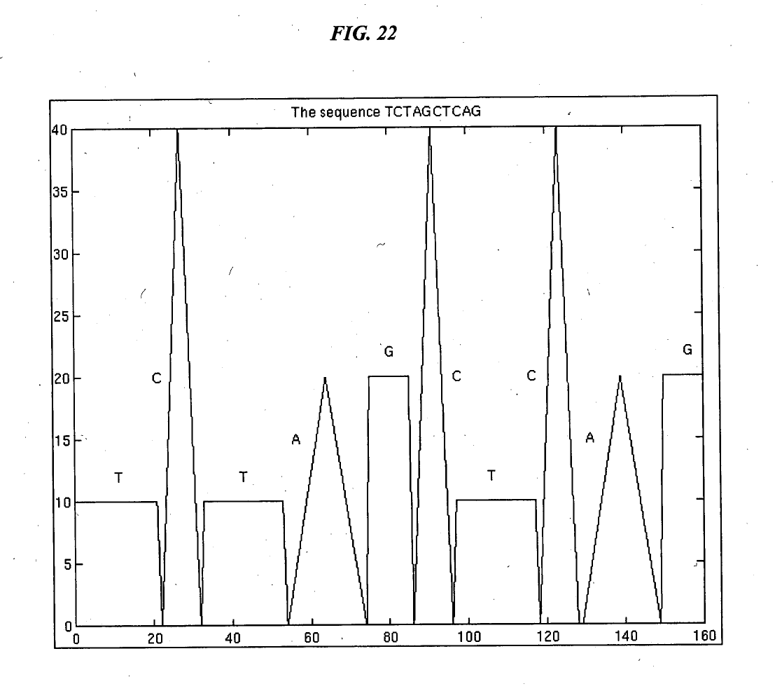

An exemplary waveform generated by the reaction of each of the bases A, C, G and T is given in FIG. 21. For purposes of illustration, the sequence of the subject riucleic acid is given as TCTAGCTCAG (SEQ ID NO:6). The resulting waveform is shown in FIG. 22. The hypothetical sampling yields the duration of the signal as RT=160.

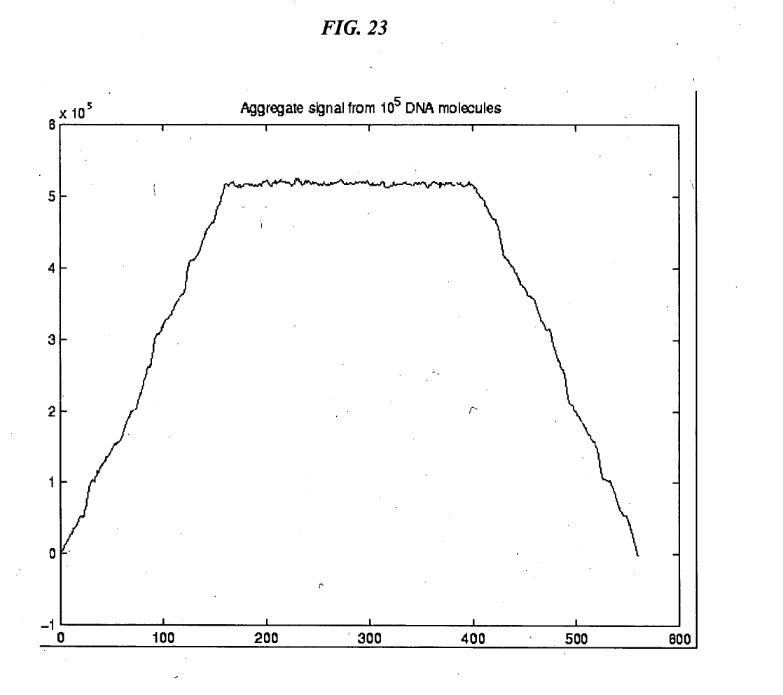

If it is assumed that there are 10⁵ such molecules in a sample and that they all react in a totally asynchronous fashion, with a uniformly distributed delay over the interval [0,400], then the noise-corrupted aggregate signal for a random run (i.e., what would be measured) is shown in FIG. 23, where D=400 and the total duration of the observed signal is D+RT=560.

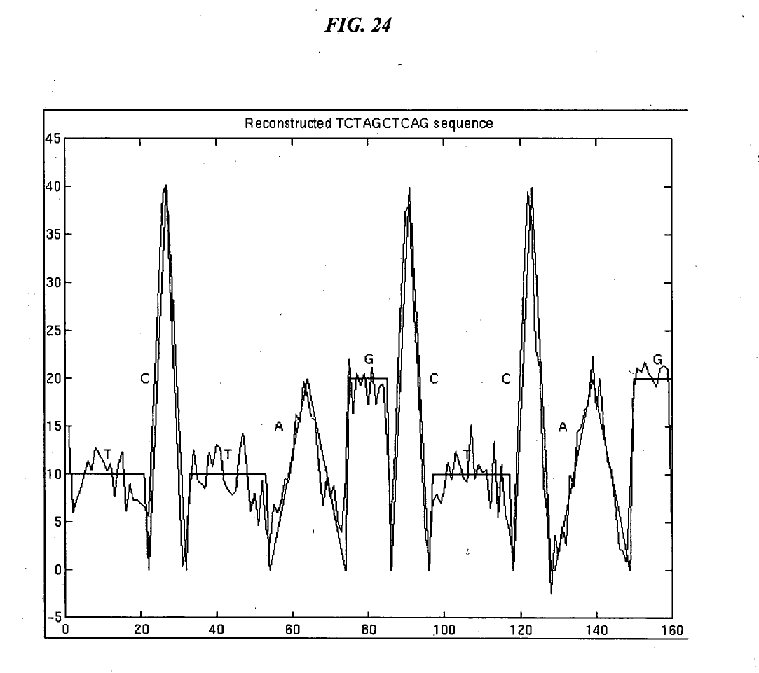

Next assume that the signal-to-noise-ratio is given by SNR_{perfect}=40 db. Due to the lack of knowledge of the Nⱼ, the effective SNR computed from Equation 11 is SNR=23.8 db. Using the Wiener solution shown in Equation 39, the recovered signature sequence is shown in FIG. 24. As can be seen, the signature signal has been successfully recovered and simple techniques, such as matched filtering, can be employed to reconstruct the sequence TCTAGCTCAG (SEQ ID NO:6).

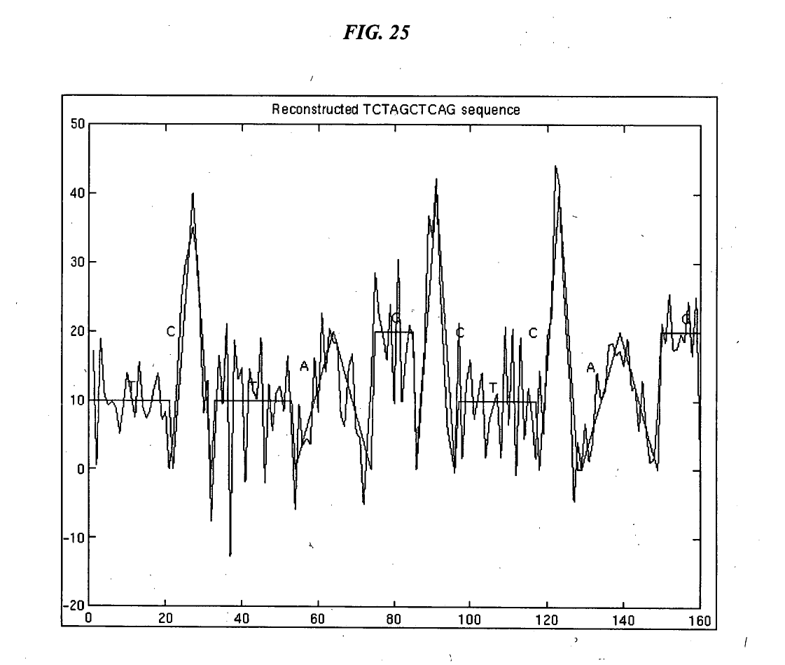

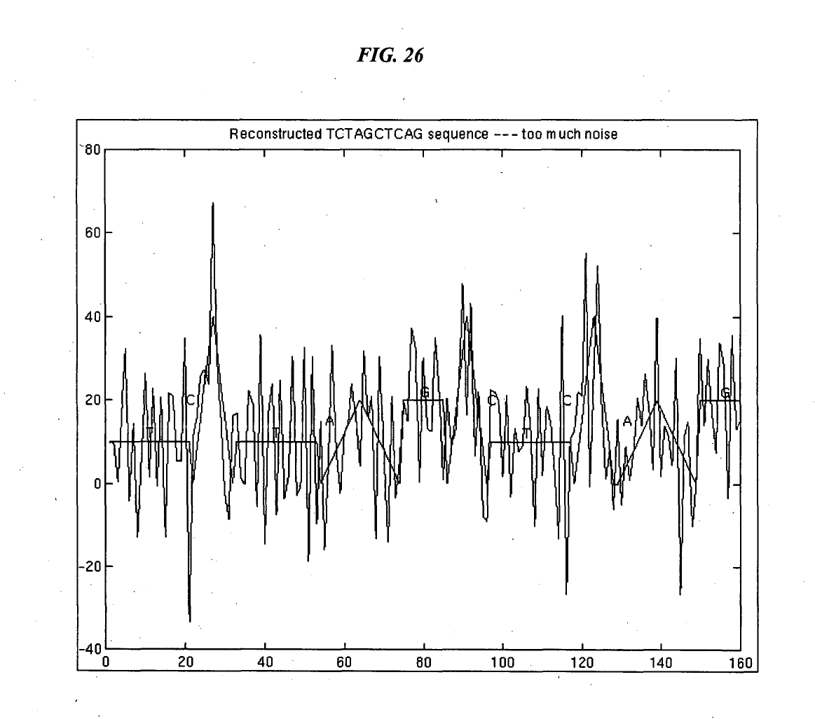

The performance of the system depends on the SNR. If the SNR is decreased to SNR_{perfect}=35 db and SNR=23.6 db then, as shown in FIG. 25, the performance degrades, although the sequence is still recoverable. However, if the SNR is further decreased to SNR_{perfect}=30 db and SNR=23 db then, as shown in FIG. 26, the sequence is barely recoverable.

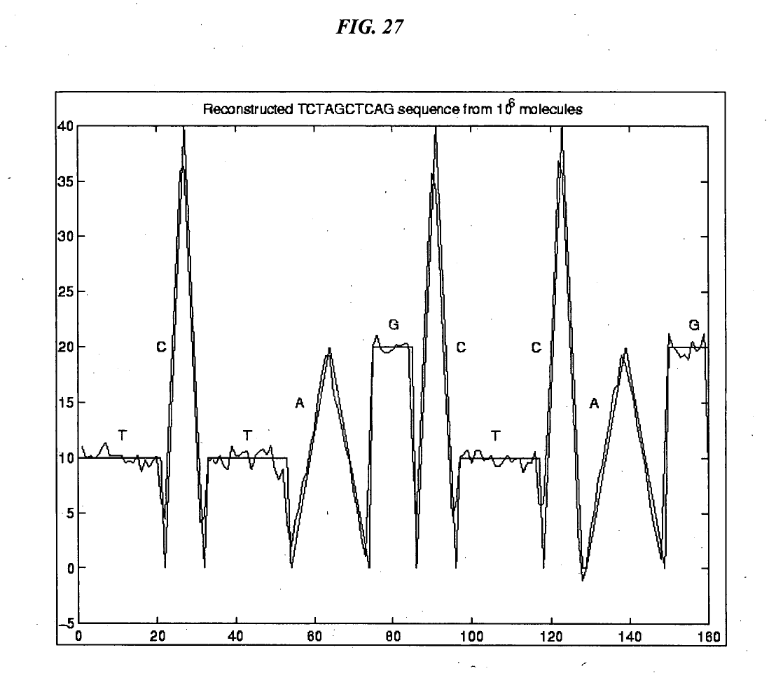

As discussed above, increasing the number of template molecules in the sample improves the performance of the system. Increasing the number of molecules to 10⁶ automatically makes the signal strength 100 times stronger. To have a fair comparison the SNR is set to SNR_{perfect}=40 db increasing the noise by 100-fold. With the new value of N we obtain SNR=28.5 db. The resulting recovered signature signal is shown in FIG. 27, which shows a clear improvement over FIG. 24.

### Example 15

### Non-Uniform Delay Distribution

Example 12 shows that using large template numbers and the Wiener solution, it is possible to recover the signature sequence from the noise-corrupted aggregate signal of its random shifts. However, the values of SNR to make this possible appear to be rather high. In the following discussion it is assumed that the duration of the delays is longer than the duration of each signature sequence, i.e., D>RT. This appears to be more appropriate for actual sequencing applications than D≦RT.

Close inspection of Equation 34 shows that, of the D+RT+1 equations, only 2RT+1 are distinct. These correspond to the first RT equations (coming from the “lower triangular” portion of the “diamond-shaped” matrix Θ) and the last RT equations (coming from the “upper triangular” portion of Θ). The D−RT+1 center equations are all identical since the corresponding rows of E are all-one row-vectors. Thus, these D−RT+1 equations all amount to a single equation giving information about

s₀+s₁+ . . . +s_{RT-1}+s_{RT},

i.e., the mean of the signature sequence. Physically, this means that information on the signature sequence can only be found in the rising transient, consisting of the first RT samples, of the observations signal and in its falling transient, consisting of the last RT samples. The middle D−RT+1 samples, corresponding to the steady-state of the observed signal, contain no information on the signature sequence other than its mean. (These three phases can be clearly seen in FIG. 23, for example.) Of the N DNA molecules only those that begin (end) their reaction during the first (last) RT samples provide information on the signature sequence. The information carried by the remaining DNA molecules gets “averaged-out” during the steady-state of the observations signal.

It may be possible to improve performance by obtaining information from all the DNA molecules and ensuring that the observations signal never goes to a steady-state. The most straightforward way to guarantee this is to have a non-uniform delay distribution. Thus, assume that the delay distribution is given by the probability sequence pᵢ, where

pᵢ=probability that the n th delay for an arbitrary n is kₙ=i, for i=0,1, . . . , D.

Being a probability sequence,

$${\sum }_{i = 0}^{D} {p}_{i} = 1.$$

Under this assumption the Nⱼ are random variables with

μ_{N}_{ⱼ}=ENⱼ=Npⱼ and σ_{N}_{ⱼ}²=E(Nⱼ−Npⱼ)²=Npⱼ(1−pⱼ) (41)

and

C_{N,N}_{ⱼ}=E(Nᵢ−Npᵢ)(Nⱼ−Npⱼ)=−Npᵢpⱼ. (42)

Equations 41 and 42 reduce to Equations 31 and 32 when the distribution is uniform and pᵢ=1/D for all i. It follows that

$$\frac{{σ}_{{N}_{j}}}{{μ}_{{N}_{j}}} = \sqrt{\frac{1 - {p}_{j}}{N {p}_{j}}} ,$$

so that as N goes to infinity, we may replace Nⱼ by its mean. Moreover, Equation 34 is replaced by

$$\begin{matrix}[ \begin{matrix}{y}_{0} \\ {y}_{1} \\ ⋮ \\ {y}_{D + RT - 1} \\ {y}_{D + RT}\end{matrix} ] = N [ \begin{matrix}{p}_{0} & & & \\ {p}_{1} & {p}_{0} & & \\ ⋮ & {p}_{1} & ⋰ & \\ {p}_{D} & ⋮ & ⋰ & {p}_{0} \\ & {p}_{D} & ⋮ & {p}_{1} \\ & & ⋰ & ⋮ \\ & & & {p}_{D}\end{matrix} ] [ \begin{matrix}{s}_{0} \\ {s}_{1} \\ ⋮ \\ {s}_{RT - 1} \\ {s}_{RT}\end{matrix} ] + \\ [ \begin{matrix}{\tilde{N}}_{0} & & & \\ {\tilde{N}}_{1} & {\tilde{N}}_{0} & & \\ ⋮ & {\tilde{N}}_{1} & ⋰ & \\ {\tilde{N}}_{D} & ⋮ & ⋰ & {\tilde{N}}_{0} \\ & {\tilde{N}}_{D} & ⋮ & {\tilde{N}}_{1} \\ & & ⋰ & ⋮ \\ & & & {\tilde{N}}_{D}\end{matrix} ] [ \begin{matrix}{s}_{0} \\ {s}_{1} \\ ⋮ \\ {s}_{RT - 1} \\ {s}_{RT}\end{matrix} ] + [ \begin{matrix}{v}_{0} \\ {v}_{1} \\ ⋮ \\ {v}_{D + RT - 1} \\ {v}_{D + RT}\end{matrix} ]\end{matrix} \qquad ( 43 )$$

As before R_{w}, the covariance matrix of the equivalent noise, may be computed. The “equivalent” SNR can be computed to be

$$\begin{matrix}SNR = \frac{{SNR}_{perfect}}{1 + \frac{{\sum }_{i = 0}^{D} {p}_{i}^{2}}{N {\sum }_{i = 0}^{D} {p}_{i} ( 1 - {p}_{i} )} {SNR}_{perfect}} , \\ where & ( 44 ) \\ {SNR}_{perfect} = \frac{{N}^{2} {P}_{s} {\sum }_{i = 0}^{D} {p}_{i}^{2}}{( D + RT ) {σ}_{v}^{2}} . & ( 45 )\end{matrix}$$

The Wiener solution is still given by Equation 39. The only difference is that

$$Θ = D [ \begin{matrix}{p}_{0} & & & \\ {p}_{1} & {p}_{0} & & \\ ⋮ & {p}_{1} & ⋰ & \\ {p}_{D} & ⋮ & ⋰ & {p}_{0} \\ & {p}_{D} & ⋮ & {p}_{1} \\ & & ⋰ & ⋮ \\ & & & {p}_{D}\end{matrix} ] .$$

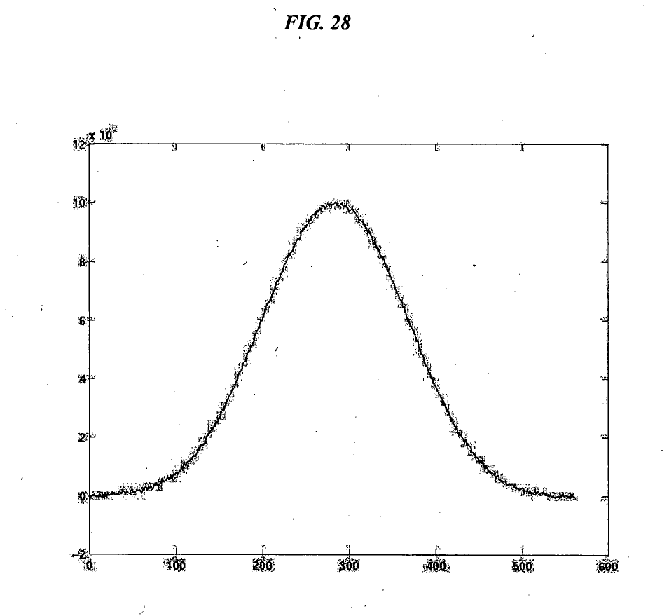

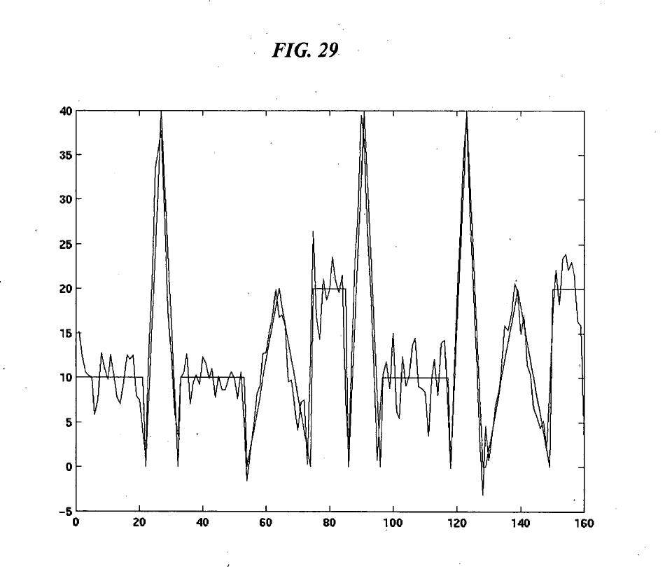

As an example of such a system, assume that the delay distribution has a Gaussian profile. Then, assuming that N=10⁵, FIG. 23 for the aggregate signal is replaced by FIG. 28, which demonstrates the absence of a steady-state. The recovered signature sequence using the Wiener solution is given in FIG. 29. The performance is comparable to that of the uniform delay case of FIG. 24.

### Example 16

### Engineering the Delay Distribution

The delay distribution has an effect on the number of DNA molecules that contribute to the recovery of the signature sequence. Although the particular Gaussian delay distribution considered in Example 13 did not appear to offer an improvement over a uniform delay distribution, there are likely to be delay distributions that provide improved results compared to uniform delay distribution.

Iteratively Estimating the Nⱼ

In the algorithm described above, the Nⱼ were estimated using statistical assumptions on the delay distribution. Once the signature sequence has been estimated based on these statistical assumptions, it is possible to rewrite Equation 29 as Equation 46 below, where the roles of the Nⱼ and sᵢ have been exchanged. With the sᵢ estimated, the coefficient matrix in Equation 46 is known and the Nⱼ may be estimated using the Wiener solution. Returning to Equation 30, the sᵢ, may be re-estimated and so on. Several iterations of this process should significantly improve the performance of the system.

$$[ \begin{matrix}{y}_{0} \\ {y}_{1} \\ ⋮ \\ {y}_{D + RT - 1} \\ {y}_{D + RT}\end{matrix} ] = [ \begin{matrix}{s}_{0} & & & \\ {s}_{1} & {s}_{0} & & \\ ⋮ & {s}_{1} & ⋰ & \\ {s}_{RT} & ⋮ & ⋰ & {s}_{0} \\ & {s}_{RT} & ⋮ & {s}_{1} \\ & & ⋰ & ⋮ \\ & & & {s}_{RT}\end{matrix} ] [ \begin{matrix}{N}_{0} \\ {N}_{1} \\ ⋮ \\ {N}_{D - 1} \\ {N}_{D}\end{matrix} ] + [ \begin{matrix}{v}_{0} \\ {v}_{1} \\ ⋮ \\ {v}_{D + RT - 1} \\ {v}_{D + RT}\end{matrix} ] , \qquad ( 46 )$$

Nonlinear Techniques:

The Wiener solution is essentially a linear technique, in the sense that the solution is linear in the observed signal yᵢ. The solution also requires no a priori knowledge of the signature sequence, i.e., that it is composed of components generated by the different base pairs A, C, G and T. This requires no assumptions on the signature sequence and so may be used to discover what the different signals generated by the A, G, C and T bases are. However, if the nature of the signature sequence is known, then it should be possible to improve the estimation. For example, if it is known that the signature sequence is composed of A, C, G and T components, each with a known response, then nonlinear techniques exist that can exploit this and improve performance. Among these are decision-feedback and maximum-likelihood techniques, the latter of which can be efficiently implemented using the Viterbi algorithm or sphere decoding.

Exploiting the Spectral Properties of the Signature Sequence:

In the Wiener solution it is not assumed that any information on the spectrum of the signature sequence is available. For example, it is reasonable to assume that the signature sequence is relatively smooth (and so devoid of high frequency components). However, the reconstructed sequences in the above Examples exhibit rapid time variations, indicating the presence of high frequency components. Therefore appropriate filtering of the recovered sequence should also improve the performance of the system.

More Complex Models

The Examples above assume that there is a unique signature sequence obtained from the DNA molecules that is repeatedly generated, with different delays. In other words, it is assumed that all molecules react in an identical fashion. However, it is possible that the actual signals may vary, depending on their context. It is plausible that the time between the reactions of one base pair to the next may be random. In this case, rather than having a unique signature sequence, there will be a distribution of signature sequences. In certain alternative embodiments one may utilize a stochastic model for the generation of the signature sequences, such as a hidden Markov model. In this case, the sequence information will be encoded in the (hidden) states of the hidden Markov Model. Many algorithms for the estimation of the state of hidden Markov models are known in the art and may be used in the practice of the claimed methods.

### Example 17

### Isothermal DNA Amplification Assays with BRC Detection

Amplification of specific DNA probes provides a powerful tool for the detection of infectious diseases, genetic diseases, and potentially cancer. Use of BRC detection may involve at least some amplification. PCR is the present amplification method of choice, but this is a time consuming and instrumentally-cumbersome step due to the requirement for temperature cycling. In certain preferred embodiments of the invention, isothermal methods of BRC detection may be used.

One method of isothermal BRC assay may involve simultaneous strand displacement amplification and real-time bioluminescence detection. Strand Displacement Amplification (SDA) is an in vitro, isothermal nucleic acid amplification technique originally based upon the ability of the restriction enzyme Hinc II to nick the unmodified strand of a hemiphosphorothioate form of its recognition site, and the ability of the 5′→3′ exonuclease-deficient Klenow fragment of DNA polymerase I (exo-klenow) to extend the 3′-end at the nick site and displace the downstream DNA strand. Exponential amplification results from coupling sense and antisense reactions in which strands displaced from a sense reaction serve as a target for an antisense extension reaction and vice versa (e.g., Walker et al., Proc. Natl. Acad. Sci 89:392-396, 1992).

Although effective, target generation by restriction enzyme cleavage presents a number of practical limitations. Little et al., (Clinical Chemistry 45:777-784, 1999) disclose an alternative approach to SDA that eliminates the requirement for restriction enzyme cleavage of the target sample prior to amplification. The method exploits the strand displacement activity of exo-klenow to generate target DNA copies with defined 5′- and 3′-ends. The new target generation process occurs at a single temperature (after initial heat denaturation of the double-stranded DNA). The target copies generated by this process are then amplified directly by SDA.

The ability of isothermal BRC to accurately detect specific DNA target sequences is demonstrated by using two different PCR amplicons, Ro 52 DNA fragment (A) and Ro 60 DNA fragment (B), with corresponding primers for each (A′ and B′).

Different buffers, with different buffer capacity, different pH values, and a spectrum of ionic strength conditions are screened, in a combinatorial fashion, for their effects on the SDA and BRC reaction steps. Currently SDA amplification is performed in a mixture containing 50 mM Tris-HCl (pH 7.4), 6 mM MgCl2, 50 mM NaCl and 50 mM KCl (9) while BRC is performed in 100 mM Tris-Acetate (pH 7.75) and 5 mM Mg-Acetate. Buffers used include Tris-acetate and Hepes-acetate buffers. The pH is varied between pH 6.5 and 8.5. The buffer concentration is varied between 0.05 M and 0.2 M. The conditions are optimized for SDA and BRC protocols.

Microwell plate wells (for placing different primer sets for individual DNA sequences) are prepared by adding primers A′, B′, a combination of A′ and B′, or an irrelevant primer set, C′ into different wells to be exposed to sample mixture. The sample mixture contains the (SDA) polymerase, luciferase, ATP sulfurylase, adenosine 5′-phosphosulfate (APS), D-luciferin (BioThema) and two different target DNA molecules, A and B. A positive signal is detected only in the wells having the appropriate DNA primers with the appropriate complementary target sequence present in the sample. The sensitivity of BRC detection technology employed with SD isothermal DNA amplification is demonstrated by employing serially diluted samples of DNA primer probes. Sensitivity in the range of 0.1 amol to 1 μmol is observed.

### Example 18

### Portable Biosensor

Certain embodiments of the invention concern a portable, photodiode-based sensor system for ultra-sensitive detection of nucleic acid molecules. The BRC chemistry has shown a high performance in terms of sensitivity and signal level. This high gain eliminates the necessity for an expensive photodetector (e.g., a cooled CCD). Maintenance of a controlled environment in the device facilitates the reliable measurement of the photon generation rate of the assay and quantification of nucleic acid molecules. A reaction chamber with controllable temperature and minimum background light is preferred.

The detector is less expensive than current molecular detection platforms, which are often sophisticated, delicate and bulky devices that are highly labor intensive. The associated biochemical procedures are expensive, require skilled personnel, and often take days or weeks to complete. BRC in combination with the handheld device is a preferred detection system, due to its low cost and higher sensitivity. This places the device within reach of many more individual users, instead of only those with access to well-equipped core facilities. In addition, the platform enables physicians and first responders to a medical emergency to diagnose problems in a rapid, sensitive, and highly specific manner, facilitating appropriate prompt response or treatment. The device can also be used for consumer and industry-based environmental monitoring, for use in healthcare and agriculture-food sectors, and for defense and homeland security in applications requiring the detection and identification of biological agents.

Photodiode and Sensor Design

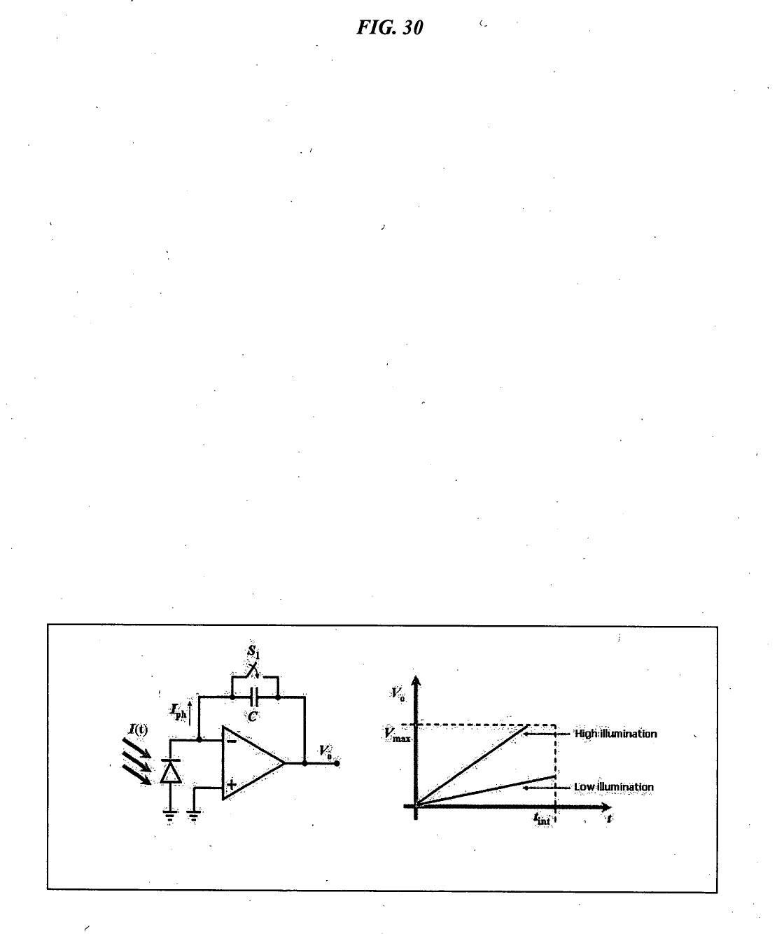

Maximization of the signal to noise ratio (SNR) of the photogenerated signal is facilitated by an understanding of photodiode and sensor parameters. Most visible light sensors comprise a 2D photodetector array, which is divided into pixels. Independent of its topology and sizing, the array contains a number of photodiodes, with a circuit as shown in FIG. 30. Photons incident on the photodiode are converted to a photocurrent, which is integrated over the capacitance C. The amount of charge collected is proportional to the light intensity and it might be clipped by saturation in high illumination. At the end of exposure time (tᵢₙₜ) the potential level is read as an electrical voltage signal (Vₒ) which is defined by

$${V}_{o} = \frac{{I}_{p h}}{qC} \cdot {t}_{int} , \qquad ( 47 )$$

where q is the charge on an electron. For the BRC assay, assuming that there is 100% light collection efficiency and photodiode with unity spectral response in the emission spectra the output potential is

$${V}_{o} = ( \frac{{t}_{int}}{qC} ) \cdot ( \frac{α \cdot {k}_{L}}{V} ) \cdot {N}_{NA} \cdot ( {L}_{NA} - {L}_{P} ) . \qquad ( 48 )$$

Several sources contribute to noise during the collection of the photogenerated signal. The shot noise generated during integration can be modeled as a Gaussian noise source with zero mean and variance of (C·q)⁻¹·(1_{dc}+1(t))·tᵢₙₜ. Other sources include read noise; reset noise and shot noise from background light and photodiode dark current (not considered here). The Signal-to-Noise Ratio, SNR, is defined as the ratio between the photogenerated signal power to the noise power and is given by

$$( \frac{S}{N} ) = \frac{{I ( t )}^{2} \cdot {t}_{int}}{q \cdot [ I ( t ) + {I}_{d c} ]} . \qquad ( 49 )$$

In order to achieve a relatively high SNR, the integration time should be increased. For the design and optimization of the sensor, the following factors are taken into account: the characteristics of the amplifier, leakage currents of the devices and analog switches, and the thermal drift of the components. Furthermore, the power supply or the type of battery which drives the circuitry and electronics is chosen with care.

Temperature Control and Thermo-Electric (TE) Cooling/Heating

Peltier devices, also known as thermoelectric (TE) modules, are small solid-state devices that function as heat pumps. It is a sandwich formed by two ceramic plates with an array of small Bismuth Telluride cubes (“couples”) in between. When a DC current is applied, heat is moved from one side of the device to the other, at which point it must be removed with a heat sink. The “cold” side is commonly used to cool an electronic device such as a microprocessor or a photodetector. If the current is reversed the device makes an excellent heater.

In some embodiments of the invention, TE heating is used to increase the temperature of the assay in the annealing and hybridization phase. Because Peltier devices can also be cooled by simply reversing the polarity of the current, it can also be used to decrease the temperature quickly (in contrast to typical resistive heaters).

Peltier devices may be controlled by a variety of different techniques such as pulse width modulation schemes. They can be stacked to achieve higher (or lower) temperatures, although reaching cryogenic temperatures would require great care. They are not very “efficient” and can draw amps of power. This disadvantage is more than offset by the advantages of non-moving parts, no vibration, very small size, long life, and capability of precision temperature control.

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method comprising:
a) obtaining at least one sample suspected of containing one or more target biomolecules;
b) using the target biomolecule to generate pyrophosphate (PPi) by an enzyme-catalyzed reaction;
c) using the PPi to produce light by a luciferase dependent process;
d) accumulating the number of photons produced over a time interval; and
e) detecting the target biomolecules in the sample.

2. The method of claim 1, wherein the luciferase dependent process comprises a bioluminescence regenerative cycle (BRC).

3. The method of claim 1, wherein PPi is converted to ATP.

4. The method of claim 3, wherein the formation of ATP from PPi is catalyzed by an enzyme selected from the group consisting of ATP sulfurylase, FMN adenyltransferase, adenylyl transferase and glucose-1-phosphate adenyltransferase.

5. The method of claim 1, wherein the target biomolecule is a nucleic acid or oligonucleotide.

6. The method of claim 5, wherein the target nucleic acid is amplified.

7. The method of claim 6, wherein the nucleic acid amplification technique is selected from the group consisting of polymerase chain reaction (PCR) amplification, strand displacement amplification, Qbeta replication, transcription-based amplification (TAS), nucleic acid sequence based amplification (NASBA), one-sided PCR, RACE (rapid amplification or cDNA ends), ligase chain reaction amplification (LCR), 3SR (self-sustained sequence replication-reaction) amplification and rolling circle replication.

8. The method of claim 7, further comprising real time PCR amplification.

9. The method of claim 1, further comprising attaching an aptamer, a nucleic acid or oligonucleotide tag to a protein.

10. The method of claim 9, wherein the protein is a target biomolecule.

11. The method of claim 9, wherein the protein is an antibody, antibody fragment, FAb fragment, genetically engineered antibody, monoclonal antibody, polyclonal antibody or single chain antibody, fusion protein, binding protein, receptor protein, enzyme, inhibitory protein or regulatory protein.

12. The method of claim 11, wherein the protein binds to a target biomolecule.

13. The method of claim 12, further comprising measuring protein-protein binding.

14. The method of claim 1, further comprising measuring gene expression levels in a sample from a cell line, tissue, organ or subject.

15. The method of claim 3, wherein the concentrations of ATP and PPi reach steady state levels.

16. The method of claim 15, further comprising integrating the light output over time during the steady state.

17. The method of claim 15, further comprising adding between 0.01 and 10 attomoles of ATP or PPi to the sample before light is produced.

18. The method of claim 1, wherein the enzyme used to generate PPi is selected from the group consisting of a DNA polymerase, an RNA polymerase, a reverse transcriptase and a terminal transferase.

19. The method of claim 18, wherein the enzyme is thermostable.

20. The method of claim 19, further comprising using thermostable luciferase to generate light.

21. The method of claim 20, further comprising using thermostable ATP sulfurylase to produce ATP from PPi.

22. The method of claim 21, wherein the luciferase, ATP sulfurylase, DNA polymerase, RNA polymerase, reverse transcriptase and/or terminal transferase remain active after exposure to at least 90° C. for at least 10 minutes.

23. The method of claim 22, wherein at least one percent of the initial activity of luciferase, ATP sulfurylase, DNA polymerase, RNA polymerase, reverse transcriptase and/or terminal transferase remains after exposure to at least 90° C. for at least 10 minutes.

24. The method of claim 1, wherein sensitivity of detection is at least 0.1 attomol.

25. The method of claim 24, wherein 1000 target biomolecules can be detected in a sample.

26. The method of claim 1, further comprising determining the number of target biomolecules in the sample.

27. The method of claim 1, further comprising identifying the target biomolecules in the sample.

28. The method of claim 5, further comprising sequencing the target nucleic acid.

29. The method of claim 5, wherein the target nucleic acid is inserted into a vector.

30. The method of claim 5, further comprising detecting a single nucleotide polymorphism (SNP) in the target nucleic acid.

31. A method of nucleic acid sequencing comprising:
a) replicating multiple copies of a nucleic acid template molecule;
b) obtaining a complex signal from the nucleic acid replication;
c) deconvoluting the signal by statistical signal processing; and
d) determining the sequence of the template nucleic acid.

32. The method of claim 31, wherein the complex signal is obtained by BRC detection.

33. The method of claim 31, wherein the signal deconvolution comprises performing a Wiener solution analysis.

34. The method of claim 33, wherein the complex signal reflects a uniformly distributed time delay.

35. The method of claim 31, wherein the delay-time distribution is engineered to improve performance.

36. The method of claim 31, further comprising iteratively estimating Nⱼ.

37. The method of claim 31, wherein the signal is deconvoluted using nonlinear techniques.

38. The method of claim 37, wherein the nonlinear techniques include maximum likelihood detection and/or sphere decoding.

39. The method of claim 31, wherein the signal sequence is modeled as the output of a hidden Markov model and deconvolution is performed using state estimation techniques.

40. A method comprising:
a) obtaining at least one sample suspected of containing one or more target proteins and/or peptides;
b) obtaining an assay mixture with at least one enzyme or substrate inactivated by peptide linkage;
c) exposing the sample to the assay mixture;
d) activating the inactivated enzyme or substrate;
e) generating pyrophosphate (PPi); and
f) producing light by a luciferase dependent process.

41. The method of claim 40, wherein the target protein is a protease and said protease removes the linked peptide from the inactivated enzyme or substrate.

42. The method of claim 40, wherein the inactivated enzyme is luciferase or ATP sulfurylase.

43. The method of claim 40, wherein the inactivated substrate is luciferin or APS.

44. The method of claim 40, further comprising measuring protein-protein binding.

45. The method of claim 40, wherein the luciferase dependent process comprises BRC.

46. A method comprising:
a) obtaining at least one sample suspected of containing one or more target nucleic acids;
b) generating pyrophosphate (PPi) by terminal transferase activity;
c) measuring the pyrophosphate generated; and
e) detecting the target nucleic acid.

47. The method of claim 46, wherein the PPi is measured by BRC assay.

48. The method of claim 47, wherein the terminal transferase reaction is terminated before the BRC assay.

49. The method of claim 47, wherein the terminal transferase reaction occurs simultaneously with the BRC assay.

50. The method of claim 46, wherein the terminal transferase is a thermostable terminal transferase.

51. A method for biomolecule detection comprising:
a) generating pyrophosphate in a biomolecule dependent process;
b) using thermostable ATP sulfurylase and luciferase to produce light from the pyrophosphate; and
c) measuring the light output to detect the biomolecule.

52. The method of claim 51, wherein the biomolecule is an oligonucleotide, polynucleotide or nucleic acid.

53. The method of claim 51, wherein the biomolecule dependent process comprises DNA polymerase activity, polymerase chain reaction amplification (PCR™), real time PCR, reverse transcriptase activity or terminal transferase activity.

54. The method of claim 51, wherein the biomolecule is a protein, peptide, antibody, antibody fragment, enzyme, receptor protein, ligand, substrate or inhibitor.

55. The method of claim 54, wherein the biomolecule is attached to an oligonucleotide.

56. A method comprising:
a) obtaining at least one sample suspected of containing one or more target nucleic acids;
b) adding labeled nucleotides to the one or more target nucleic acids with a thermostable terminal transferase; and
c) detecting the labeled nucleic acids.

57. The method of claim 56, wherein each type of nucleotide is labeled with a distinguishable label.

58. The method of claim 57, wherein the nucleotides are labeled with one or more fluorophores.

59. A method of biomolecule detection comprising:
a) attaching a target molecule to a substrate;
b) binding a first binding moiety to the target molecule;
c) binding a second binding moiety to the first binding moiety, wherein the second binding moiety is attached to a dextran or dendromer molecule labeled with oligonucleotides;
d) generating pyrophosphate by terminal transferase mediated addition of nucleotides to the oligonucleotides; and
e) detecting the pyrophosphate.

60. The method of claim 59, wherein the pyrophosphate is detected by BRC assay.

61. A system comprising:
a) one or more reaction chambers;
b) a microfluidic system;
c) one or more photodectectors d) a thermostable luciferase; and
e) a thermostable ATP sulfurylase.

62. The method of claim 1, further comprising binding an aptamer to the target biomolecule.

63. The method of claim 62, further comprising detecting the bound aptamer.


SEQUENCE LISTING (6 sequences)

SEQ ID NO: 1 - DNA, 21 bp, Artificial
  Synthetic Oligonucleotide
cggcgataaaggctataacgg

SEQ ID NO: 2 - DNA, 21 bp, Artificial
  Synthetic Oligonucleotide
cggcgataaaggctataacgg

SEQ ID NO: 3 - DNA, 20 bp, Artificial
  Synthetic Oligonucleotide
ctggaacgctttgtccgggg

SEQ ID NO: 4 - DNA, 20 bp, Artificial
  Synthetic Oligonucleotide
ctggaacgctttgtccgggg

SEQ ID NO: 5 - DNA, 76 bp, Artificial
  Synthetic Oligonucleotide
ttttttttttttttttttttgctggaattcgtcagactggccgtcgttttacaacggaacggcagcaaaatgttgc

SEQ ID NO: 6 - DNA, 10 bp, Artificial
  Synthetic Oligonucleotide
tctagctcag